(19) United States
(12) Patent Application Publication
(10) Pub. No.: US 2023/0028062 A1
(43) Pub. Date: Jan. 26, 2023

(54) METHODS FOR ENHANCING IMMUNITY AND TUMOR TREATMENT

(71) Applicant: Rakuten Medical, Inc., San Diego, CA (US)

(72) Inventors: Miguel GARCIA-GUZMAN, San Diego, CA (US); Jerry FONG, San Diego, CA (US); Amy THORNE, San Diego, CA (US); Michelle HSU, Rochester, MN (US)

(73) Assignee: Rakuten Medical, Inc., San Diego, CA (US)

(21) Appl. No.: 17/782,111

(22) PCT Filed: Dec. 4, 2020
(86) PCT No.: PCT/US2020/063444

§ 371 Date: Jun. 2, 2022

Related U.S. Application Data
(60) Provisional application No. 62/945,053, filed on Dec. 6, 2019.

(51) Int. Cl. A61K 41/00; A61K 47/68; A61P 35/00

(52) U.S. Cl. CPC A61K 41/0071; A61K 47/6849; A61P 35/00

(57) ABSTRACT

Provided are compositions, combinations, and methods and uses for treating a subject having a tumor, lesion or cancer. In some aspects, the methods and uses include administering a targeting molecule that binds PD-L1, conjugated with phthalocyanine dye, such as IR700. In some aspects, after administration of the conjugate, a target area is illuminated with a wavelength of light suitable for the activation of the conjugate. In some aspects, the illumination leads to the killing of PD-L1-expressing cells. The provided embodiments result in growth inhibition, volume reduction, and elimination of tumors, lesions or cancers, including metastatic tumor cells, invasive tumor cells, heterogeneous tumors and/or tumors that are not responsive to and/or resistant to other therapies. The disclosure also relates to compositions, combinations, methods and uses for enhancing immune responses, such as anti-tumor or anti-cancer immune responses, for responses against tumor growth and for effective treatment of tumors, lesions or cancers.

### RELATED APPLICATIONS

This application claims priority from U.S. Provisional Application No. 62/945,053, filed Dec. 6, 2019, entitled “METHODS FOR ENHANCING IMMUNITY AND TUMOR TREATMENT,” the contents of which are incorporated by reference in their entirety.

### FIELD

The present disclosure relates to compositions, combinations, and methods and uses for treating a subject having a tumor, a lesion or a cancer. In some aspects, the methods include administering to the subject a targeting molecule that binds PD-L1, conjugated with phthalocyanine dye, such as IR700. In some aspects, after administration of the targeting molecule-phthalocyanine dye conjugate, a target area is illuminated with a wavelength of light suitable for the activation of the phthalocyanine dye of the conjugate. In some aspects, the illumination leads to cell killing of cells that express PD-L1. The provided embodiments result in growth inhibition, volume reduction, and elimination of tumors, lesions or cancers, including metastatic tumor cells, invasive tumor cells, heterogeneous tumors and/or tumors that are resistant to other therapies. The disclosure also relates to compositions, combinations, methods and uses for enhancing immune responses, such as anti-tumor or anti-cancer immune responses, and for responses against tumor growth and effective treatment of tumors, lesions or cancers.

### BACKGROUND

Every year many therapeutics for treating cancer are developed, including immune checkpoint inhibitors, small molecule targeted therapies, and other anticancer therapeutics. However, some patients are not responsive to those therapeutics, and a majority of cancer patients will eventually develop non-responsiveness or resistance to therapeutics they receive during their treatment courses, leading to disease progression and cancer-related deaths. Novel compositions and methods are urgently needed to address these clinical challenges.

### SUMMARY

Provided herein are methods and uses for treating a tumor or a lesion in a subject by activating an immune cell response that involves administering to a subject having a tumor or a lesion a conjugate comprising a phthalocyanine dye linked to a targeting molecule that binds to PD-L1. In some of any embodiments, the conjugate comprises an antibody that binds to PD-L1 and a phthalocyanine dye IR700. In some of any embodiments, the methods also involve illuminating a target site in which PD-L1 expressing cells are located. For example, in some of any embodiments, the target area is illuminated at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the methods and uses result in or lead to killing of cells that express PD-L1. In some of any embodiments, the methods and uses result in or leads to the reduction or inhibition growth of the tumor or the lesion, and/or reduction or inhibition of tumor metastasis and/or newly arising tumors.

Also provided herein are methods and uses for treating a tumor or a lesion in a subject by activating an immune cell response that involves: administering to a subject having a tumor or a lesion a conjugate comprising a phthalocyanine dye linked to a targeting molecule that binds to PD-L1; and illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located. In some of any embodiments, the methods or uses result in the killing of the PD-L1 expressing immune cell and thereby inhibits the growth of the tumor or the lesion.

Also provided herein are methods and uses for treating a tumor or a lesion in a subject that involve: administering a conjugate comprising a phthalocyanine dye linked to a targeting molecule that binds to PD-L1 to a subject having a tumor or lesion comprising a tumor cell that is reduced in susceptibility to treatment with an immune checkpoint inhibitor; and illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length; wherein after the illumination, the growth, size or viability of the tumor or lesion is reduced or inhibited.

Also provided herein are methods and uses for treating a tumor or lesion that involve: administering a conjugate comprising a phthalocyanine dye linked to a targeting molecule that binds to PD-L1 to a subject having a tumor or lesion that has had a low response to, was unresponsive to, was resistant to, was refractory to, had failed to respond to or has relapsed after, a prior immunotherapy; and illuminating a target area where the tumor or lesion is located, at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the methods and uses result in the killing of a PD-L1 expressing cell in the target area.

Also provided herein are methods and uses for treating a subject having a low response or that is unresponsive to a prior immunotherapy for a tumor or a lesion. In some of any embodiments, the methods and uses involve: identifying a subject having a low response or that is unresponsive to a prior immunotherapy for a tumor or a lesion; (b) administering to a subject having a tumor or a lesion that has had a low response or that was unresponsive to a prior immunotherapy a conjugate comprising a phthalocyanine dye linked to a targeting molecule that binds to PD-L1; and (c) illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where PD-L1 expressing cells are located. In some of any embodiments, the methods or uses result in the killing of a PD-L1 expressing cell and thereby increases the number or activity of immune cells in the tumor and/or in the tumor microenvironment.

Also provided herein are methods and uses for treating a subject having a low response or that is unresponsive to a prior immunotherapy for a tumor or a lesion that involve: identifying a subject that has had a low response to, was unresponsive to, was resistant/refractory to, had failed to respond to or has relapsed after to a prior immunotherapy for a tumor or a lesion; administering to a subject having a tumor or a lesion that has had a low response to, was unresponsive to, was resistant/refractory to, had failed to respond to or has relapsed after to a prior immunotherapy a conjugate comprising a phthalocyanine dye linked to a targeting molecule that binds to PD-L1; and illuminating a target area where PD-L1 expressing cells are located. In some of any embodiments, the illuminating is carried out at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the methods or uses result in the killing of a PD-L1 expressing cell and thereby increases the number or activity of immune cells in the tumor and/or in the tumor microenvironment.

Also provided herein are methods and uses for enhancing a response to an anti-cancer agent in a subject having a tumor or a lesion that involve: administering an anti-cancer agent to a subject having a tumor or a lesion; administering a conjugate comprising a phthalocyanine dye linked to a targeting molecule that binds to PD-L1 to the subject; and illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located; wherein the methods or uses result in a greater inhibition of growth of the tumor or the lesion compared to the inhibition by treatment with the anti-cancer agent alone.

Also provided herein are methods and uses for enhancing a response to an anti-cancer agent in a subject having a tumor or a lesion that involve: administering to a subject a conjugate comprising a phthalocyanine dye linked to a targeting molecule that binds to PD-L1 to the subject; and illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, wherein the subject had been administered an anti-cancer agent, and wherein the methods or uses result in a greater inhibition of growth of the tumor or the lesion compared to the inhibition by treatment with the anti-cancer agent alone.

Also provided herein are methods and uses for enhancing a response to an anti-cancer agent in a subject having a tumor or a lesion that involve: administering an anti-cancer agent to a subject; wherein the subject had received a treatment comprising administering a conjugate comprising a phthalocyanine dye linked to a targeting molecule that binds to PD-L1 to the subject; and illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, and wherein the methods or uses result in a greater inhibition of growth of the tumor or the lesion compared to the inhibition by treatment with the anti-cancer agent alone.

Also provided herein are methods and uses for immunizing a subject having a first tumor or lesion that involve: administering a conjugate comprising a phthalocyanine dye linked to a targeting molecule that binds to PD-L1 to a subject having a tumor or lesion; and illuminating a target area within the first tumor or lesion. In some of any embodiments, the illuminating is carried out at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, wherein the first tumor or lesion is inhibited in growth and/or reduced in size; and the appearance, growth or establishment of one or more second tumors or lesions, located distally to the treated first tumor or lesion, is inhibited, delayed or prevented.

Also provided herein are methods and uses for enhancing an innate immune response subject having a tumor or a lesion that involve: administering a conjugate comprising a phthalocyanine dye linked to a targeting molecule that binds to PD-L1 to the subject; and illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the innate immune response of the subject is enhanced.

Also provided herein are methods and uses for increasing the number or level of immune cells in a tumor or a lesion that involve: administering a conjugate comprising a phthalocyanine dye linked to a targeting molecule that binds to PD-L1 to a subject having a tumor or lesion; and illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the number or level of immune cells in the tumor or lesion in the subject is increased.

Also provided herein are methods and uses for treating a heterogeneous tumor or lesion that involve: administering a conjugate comprising a phthalocyanine dye linked to a targeting molecule that binds to PD-L1 to a subject having a tumor or lesion; and illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the heterogeneous tumor or lesion in the subject is treated. In some of any embodiments, the tumor or lesion contains a plurality of different types of tumor cells or tumor cells from a plurality of different origins.

Also provided herein are methods treating an immunosuppressive tumor or lesion that involve: administering a conjugate comprising a phthalocyanine dye linked to a targeting molecule that binds to PD-L1 to a subject having a tumor or lesion; and illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the immunosuppressive tumor or lesion in the subject is treated.

Also provided herein are methods and uses for vaccinating a subject to generate an anti-cancer immune response, that involve: administering a conjugate comprising a phthalocyanine dye linked to a targeting molecule that binds to PD-L1 to a subject; and illuminating a target area; wherein the methods or uses result in an anti-cancer response selected from a delay or inhibition in the appearance of or growth of a tumor in the subject or an appearance or increase in T memory cells in the vicinity of a tumor. In some of any embodiments, the illuminating is carried out at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, the methods or uses result in the killing of the PD-L1 expressing cell or the PD-L1 expressing immune cell.

Also provided herein are methods and uses for administering a conjugate that involve: administering a phthalocyanine dye linked to a targeting molecule that binds to PD-L1 to a subject that is treatment-naïve for an immune checkpoint inhibitor or that has not previously received a treatment with an immune checkpoint inhibitor; and illuminating a target area where a tumor or lesion is located in the subject. In some of any embodiments, the illuminating is carried out at a wavelength of at or about 600 nm to at or about 850 nm and at a dose of from at or about 25 J/cm² to at or about 400 J/cm² or from at or about 2 J/cm fiber length to at or about 500 J/cm fiber length. In some of any embodiments, after the illumination, the growth, size or viability of the tumor or lesion is reduced or inhibited.

In some of any embodiments, the target area comprises PD-L1 expressing cells. In some of any embodiments, the PD-L1 expressing cell is an immune cell. In some of any embodiments, the target area comprises immune cells expressing PD-L1. In some of any embodiments, the PD-L1 expressing cell is an immune cell.

In some of any embodiments, the enhancing the innate immune response comprises an increase in activated dendritic cells (DC) or antigen-presenting dendritic cells. In some of any embodiments, the activated DCs exhibit a cell surface phenotype of CD80+ and/or CD40+. In some of any embodiments, the antigen-presenting dendritic cells exhibit a cell surface phenotype of CD11b+CD103+ CD11c+.

In some of any embodiments, the immune cell is an intratumoral neutrophil. In some of any embodiments, the intratumoral neutrophil exhibits a cell surface phenotype of CD11b⁺Ly6C^{-/low} Ly6G+. In some of any embodiments, the immune cell is an intratumoral effector T cell. In some of any embodiments, the intratumoral effector T cell exhibits a cell surface phenotype of CD3⁺ CD8⁺ PD-1⁻.

In some of any embodiments, the prior immunotherapy is a treatment with an immune checkpoint inhibitor. In some of any embodiments, the subject has primary resistance or acquired resistance to a prior immunotherapy that comprises PD-1/PD-L1 blockade.

In some of any embodiments, immunosuppressive tumor or lesion comprises a tumor cell that expresses an immune checkpoint protein. In some of any embodiments, the immune checkpoint protein is PD-L1, PD-1 or CTLA-4.

In some of any embodiments, the anti-cancer agent is selected from a checkpoint inhibitor, an immune adjuvant, a chemotherapeutic agent, radiation, and a biologic comprising an anti-cancer targeting molecule that binds to a tumor cell. In some of any embodiments, the anti-cancer agent is an antibody conjugate. In some of any embodiments, the antibody conjugate comprises a phthalocyanine dye, a toxin, or a TLR agonist.

In some of any embodiments, the subject is administered the anti-PD-L1 conjugate to treat and/or inhibit the growth of a first tumor or a first lesion; and the method inhibits or delays the appearance of one or more second tumors or lesions or a metastasis of the first tumor or the first lesion. In some of any embodiments, the one or more second tumor is phenotypically and/or genotypically different from the first tumor. In some of any embodiments, the one or more second tumor is not derived from a metastasis of the first tumor. In some of any embodiments, treatment delays regrowth of the tumor or lesion, prevents a relapse of a cancer associated with the tumor or lesion or prolongs the duration of remission of a cancer associated with the tumor or lesion.

In some of any embodiments, the PD-L1 expressing immune cell is selected from the group consisting of monocytes, macrophages, dendritic cells (DC), M2 tumor associated macrophages (M2 TAM), tolerogenic dendritic cells (tDC) and myeloid derived suppressor cells (MDSC). In some of any embodiments, the PD-L1 expressing immune cell is located in the tumor, the tumor microenvironment or a lymph node. In some of any embodiments, the tumor or the lesion comprises PD-L1 negative tumor cells. In some of any embodiments, more than at or about 40%, 50%, 60%, 70%, 80%, 90% or 95% of the tumor cells in the tumor or the lesion are PD-L1 negative tumor cells. In some of any embodiments, the tumor cell is not specifically recognized by an anti-PD-L1 antibody. In some of any embodiments, the tumor cell does not express or has a reduced expression of an immune checkpoint protein. In some of any embodiments, the immune checkpoint protein is selected from among PD-L1, PD-1, and CTLA-4. In some of any embodiments, the tumor cell does not express PD-L1 in response to an inflammatory stimulus. In some of any embodiments, the inflammatory stimulus is interferon.

In some of any embodiments, the tumor or lesion is resistant to an anti-PD-L1 therapy. In some of any embodiments, the anti-PD-L1 therapy is a treatment with an anti-PD-L1 antibody. In some of any embodiments, the methods described herein involve or result in a greater inhibition of the growth, size or viability of the tumor or lesion as compared to the inhibition by anti-PD-L1 therapy. In some of any embodiments, the inhibition of the growth of the tumor or lesion and/or killing of the PD-L1 expressing cell is dependent on the presence of CD8+ T cells.

In some of any embodiments, the subject is administered the conjugate to treat and/or inhibit the growth of and/or reduce the size of a first tumor or lesion. In some of any embodiments, the methods and uses inhibit, delay or prevent the appearance, growth or establishment of one or more second tumors or lesions, located distally to the first tumor or lesion.

In some of any embodiments, the subject has been previously treated with an anti-cancer treatment and/or an immune checkpoint inhibitor. In some of any embodiments, the subject has been previously treated with an immune checkpoint inhibitor. In some of any embodiments, the subject has had a low response to, was unresponsive to, was resistant/refractory to, failed to respond to or has relapsed after the previous treatment with the anti-cancer treatment and/or immune checkpoint inhibitor. In some of any embodiments, the subject has had a low response to, was unresponsive to, was resistant/refractory to, failed to respond to or has relapsed after the previous treatment with the immune checkpoint inhibitor. In some of any embodiments, the inhibition of tumor growth resulting from carrying out the method or use is greater compared to the inhibition of tumor growth as a result of the previous treatment with the anti-cancer treatment and/or immune checkpoint inhibitor. In some of any embodiments, the inhibition of tumor growth resulting from carrying out the method or use is greater compared to the inhibition of tumor growth as a result of the previous treatment with the immune checkpoint inhibitor.

In some of any embodiments, the growth or establishment of a second tumor or lesion, located distally to the treated first tumor or lesion, is inhibited or prevented. In some of any embodiments, the second tumor or lesion is a metastasis of the first tumor or lesion. In some of any embodiments, the methods described herein involve or result in killing of a PD-L1-expressing cell in the vicinity of the first tumor or lesion and/or activates an immune cell response, thereby inhibiting or preventing the growth of the second tumor or lesion. In some of any embodiments, the second tumor or lesion is phenotypically and/or genotypically the same as the first tumor or lesion. In some of any embodiments, the second tumor or lesion is phenotypically and/or genotypically different from the first tumor or lesion. In some of any embodiments, the one or more second tumors or second lesions is not derived from a metastasis of the first tumor or lesion.

In some of any embodiments, the subject has been previously treated with an immune checkpoint inhibitor. In some of any embodiments, the subject has had a low response to, was unresponsive to, was resistant/refractory to, had failed to respond to or has relapsed after the previous treatment with the immune checkpoint inhibitor. In some of any embodiments, the inhibition of tumor growth resulting from carrying out the method or use is greater compared to the inhibition of tumor growth as a result of the previous treatment with the immune checkpoint inhibitor. In some of any embodiments, the immune checkpoint inhibitor is an anti-PD-L1 immunotherapy.

In some of any embodiments, the subject is naïve to treatment with an immune checkpoint inhibitor or has not previously received treatment with an immune checkpoint inhibitor. In some of any embodiments, the immune checkpoint inhibitor is an inhibitor of PD-L1, PD-1 or CTLA-4.

In some of any embodiments, immune checkpoint inhibitor is a PD-1 inhibitor. In some of any embodiments, the PD-1 inhibitor is an anti-PD-1 antibody. In some of any embodiments, the immune checkpoint inhibitor is PD-L1 inhibitor. In some of any embodiments, the PD-L1 inhibitor is an anti-PD-L1 antibody.

In some of any embodiments, the subject has a tumor or a lesion having a low number or level of CD8+ T cell infiltration. In some of any embodiments, prior to administering the conjugate, the subject has a tumor or lesion having a low number or level of CD8+ T cell infiltration. In some of any embodiments, the number, level or activity of immune cells is increased in the tumor or lesion, or in the microenvironment of the tumor or lesion, after the administering and the illuminating. In some of any embodiments, the number or level of CD8+ T cell infiltration in the tumor or lesion is increased after the administering and the illuminating. In some of any embodiments, the number or level of memory T cells in the vicinity of the tumor or lesion is increased after the administering and the illuminating.

In some of any embodiments, the targeting molecule is or comprises an antibody or antigen-binding fragment thereof. In some of any embodiments, the targeting molecule is an antibody, antibody fragment or antibody-like molecule that binds PD-L1. In some of any embodiments, the targeting molecule is or comprises an anti-PD-L1 antibody or antigen-binding fragment thereof.

In some of any embodiments, the antibody or antigen-binding fragment thereof comprises complementary determining regions (CDRs) from an antibody selected from the group consisting of atezolizumab (MPDL3280A, Tecentriq, RG7446), avelumab (Bavencio), BCD-135, BGB-A333, BMS-936559 (MDX-1105), CBT-502 (TQB-2450), cosibelimab (CK-301), CS1001 (WPB3155), durvalumab (MEDI4736, Imfinzi), FAZ053, HLX20, INBRX-105, KN035, KN046, LDP, LY3300054, LY3415244, M7824 (MSB0011359C), MCLA-145, MSB2311, NM-01, REGN3504, SHR-1316 (HTI-1088), STI-3031 (IMC-001, STI-A1015), TG-1501, ZKABOO1 (STI-A1014). In some of any embodiments, the antibody or antigen-binding fragment comprises complementary determining regions (CDRs) from atezolizumab, avelumab, durvalumab, KN035 or CK-301. In some of any embodiments, the antibody or antigen-binding fragment is selected from the group consisting of atezolizumab, avelumab, durvalumab, KN035, CK-301, or biosimilar, interchangeable, biobetter, copy biological or biogeneric thereof, or an antigen-binding fragment thereof. In some of any embodiments, the antibody or antigen-binding fragment is selected from the group consisting of atezolizumab, avelumab, durvalumab, KN035, CK-301.

In some of any embodiments, the target area is in the vicinity of a tumor or lesion. In some of any embodiments, the target area is a lymph node or in the vicinity of a lymph node.

In some of any embodiments, the subject exhibits a durable response, prolonged progression-free survival, a reduced chance of relapse, and/or a reduced chance of metastasis, after the administering and the illuminating.

In some of any embodiments, the phthalocyanine dye is a Si-phthalocyanine dye. In some of any embodiments, the Si-phthalocyanine dye is IR700.

In some of any embodiments, the illuminating is carried out between 30 minutes and 96 hours after administering the conjugate. In some of any embodiments, the illuminating is carried out 24 hours±4 hours after administering the conjugate. In some of any embodiments, the target area is illuminated at a wavelength of 690±40 nm. In some of any embodiments, the target area is illuminated at a dose of at or about of 50 J/cm² or at or about 100 J/cm of fiber length.

In some of any embodiments, the tumor or lesion is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

In some of any of the provided methods, one or more of steps of the method are repeated. In some of any embodiments, the administration of the conjugate is repeated one or more times. In some of any embodiments, after each repeated administration of the conjugate, the illuminating step is repeated. In some of any of the provided methods, the method also involves administering an additional therapeutic agent or anti-cancer treatment.

### BRIEF DESCRIPTION OF THE DRAWINGS

FIG. 1 shows the average tumor volume over time, in mice with an implanted CT26 tumor that had been administered an anti-PD-L1 antibody-IR700 conjugate, followed by illumination of light at 690 nm and a dosage of 75, 100 or 150 J/cm²(α-PD-L1-IR700+75, 100 or 150 J/cm²), without light illumination (α-PD-L1-IR700), or saline administered control. The fractions and percentages of mice achieving a complete response (CR) are also shown. Mice that achieved CR were challenged with a second tumor in FIGS. 2A-2B.

FIGS. 2A-2B show the group average tumor volume (FIG. 2A) and individual tumor volumes (FIG. 2B) over time after a challenge by implantation of a second CT26 tumor, in mice from FIG. 1 that had achieved a CR. The fractions of mice achieving a complete response (CR) are also shown. Mice that achieved CR (*: except one CR mouse from the α-PD-L1-IR700+100 J/cm² group) were challenged with a third tumor of a different type in FIGS. 3A-3B.

FIGS. 3A-3B show the group average tumor volume (FIG. 3A) and individual tumor volumes (FIG. 3B) over time after a challenge by implantation of a third 4T1-EpCAM tumor, in mice from FIGS. 2A-2B that had achieved a CR. The fractions and percentages of mice achieving a complete response (CR) are also shown.

FIG. 4 shows the average tumor volume over time, in mice with an implanted CT26 tumor that had been administered an anti-PD-L1 antibody-IR700 conjugate, followed by illumination of light at 690 nm and a dosage of 100 J/cm²(α-PD-L1-IR700 PIT), or also depleted of CD8 cells (α-PD-L1-IR700 PIT+CD8 depl.), or without light illumination (α-PD-L1-IR700), without light illumination and depleted of CD8 cells (α-PD-L1-IR700 + CD8 depl.), or saline administered control. The fractions and percentages of mice achieving a complete response (CR) are also shown.

FIGS. 5A-5F show the group average tumor volume and individual tumor volume in mice that had achieved a CR after a first round CT26 tumor and anti-PD-L1-IR700 PIT, that were challenged with a second round tumor as follows: (a) CT26 (FIGS. 5A (group average) and 5B (individual mice)), (b) 4T1.WT (parental 4T1 cells without engineering; FIGS. 5C (group average) and 5D (individual mice)), and (c) RENCA mouse renal adenocarcinoma (FIGS. 5E (group average) and 5F (individual mice)). Previously untreated (naïve mice) were challenged with CT26, 4T1.WT, or RENCA tumors as controls (FIGS. 5A-5F). Mice that achieved a CR from group (a) were challenged with a third tumor of a different type in FIGS. 6A-6B.

FIGS. 6A-6B show the group average tumor volume (FIG. 6A) and individual tumor volumes (FIG. 6B) in mice after a challenge by implantation of a third round 4T1-EpCAM tumor, in mice from FIGS. 5A-5B that had achieved a CR and had rejected a CT26 tumor challenge.

FIG. 7A shows PD-L1 expression in CT26 cells or CT26 cells with a knock-out (KO) of the gene encoding PD-L1, at a basal level (without IFNγ) or in the presence of IFNγ, or a negative control.

FIG. 7B shows the average tumor volume over time in mice with an implanted CT26 PD-L1 knock-out (KO) tumor that had been administered an anti-PD-L1 antibody-IR700 conjugate, followed by illumination of light at 690 nm and a dosage of 75, 100 or 150 J/cm² (α-PD-L1-IR700+75, 100 or 150 J/cm²), anti-PD-L1 antibody-IR700 conjugate without light illumination (α-PD-L1-IR700), or saline administered control.

FIG. 7C shows the survival of mice bearing CT26 PD-L1 KO tumors that were administered an anti-PD-L1 antibody-IR700 conjugate, followed by illumination of light at 690 nm and a dosage of 75 J/cm²(α-PD-L1 PIT (75 J/cm²)), anti-PD-L1 antibody-IR700 conjugate without light illumination (α-PD-L1-IR700), or saline (control).

FIG. 8 shows the proportion of intratumoral macrophages (CD11b+F4/80+ cells; left panel), dendritic cells (CD11c+ cells; center panel), and MDSCs (CD11b+ Ly6C+Ly6G-cells; right panel) in tumors that had been administered anti-PD-L1 antibody-IR700 conjugate, followed by illumination of light at 690 nm and a dosage of 100 J/cm² (PDL1 PIT), anti-PD-L1 antibody-IR700 conjugate without light illumination (PDL1 Conj.), or saline (control) 2 days prior.

FIG. 9 shows the proportion of intratumoral neutrophils (CD11b⁺Ly6C^{-/low} Ly6G⁺ cells) in tumors that had been administered anti-PD-L1 antibody-IR700 conjugate, followed by illumination of light at 690 nm and a dosage of 100 J/cm² (PDL1 PIT), anti-PD-L1 antibody-IR700 conjugate without light illumination (PDL1 Conj.), or saline (control) two days prior.

FIGS. 10A-10C show the proportion of dendritic cells (DCs) displaying activation markers CD80+ (FIG. 10A) and CD40+ (FIG. 10B) and antigen-presenting DCs (CD11b+CD103+ CD11c+ cells; FIG. 10C) in tumors that had been administered anti-PD-L1 antibody-IR700 conjugate, followed by illumination of light at 690 nm and a dosage of 100 J/cm² (PDL1 PIT), anti-PD-L1 antibody-IR700 conjugate without light illumination (PDL1 Conj.), or saline (control) 2 days prior.

FIGS. 11A-11C show the proportion of total CD8+ T cells (FIG. 11A), exhausted CD8+ T cells (FIG. 11B), and “newly primed” CD8+ T cells (FIG. 11C) in tumors that had been administered anti-PD-L1 antibody-IR700 conjugate, followed by illumination of light at 690 nm and a dosage of 100 J/cm² (PDL1 PIT), anti-PD-L1 antibody-IR700 conjugate without light illumination (PDL1 Conj.), or saline (control) 8 days prior.

FIG. 12 depicts the abscopal effects on distal, non-illuminated CT26 tumors over time in mice that had been administered anti-PD-L1 antibody-IR700 conjugate without illumination (anti-PD-L1-IR700 conj.), anti-PD-L1 conjugate followed by illumination at 690 nm and a dosage of 100 J/cm² on a tumor implanted on the contralateral side (anti-PD-L1 PIT), or saline control.

FIGS. 13A-13C show the average tumor volume (FIG. 13A), individual tumor volumes (FIG. 13B), and survival (FIG. 13C) over time in mice with an implanted CT26 tumor that had been administered an anti-PD-L1 antibody-IR700 conjugate, followed by illumination of light at 690 nm and a dosage of 75 J/cm²(α-PD-L1 PIT), anti-PD-L1 conjugate without illumination (α-PD-L1-IR700 conj.), bi-weekly doses of naked (unconjugated) anti-PD-L1 antibody (α-PD-L1 multi-dosing; indicated by arrows under x-axis), or saline (control). The fractions of mice achieving a complete response (CR) are also shown in FIGS. 13A and 13B.

FIGS. 14A and 14B show the average tumor volume over time in mice with an implanted LL/2 murine lung carcinoma tumor that had been administered naked anti-PD-1 antibody, naked anti-CTLA-4 antibody, or saline (FIG. 14A; antibody dosing indicated by arrows under x-axis) or anti-PD-L1-IR700 conjugate, followed by illumination of light at 690 nm and a dosage of 150 J/cm² (anti-PD-L1 PIT), anti-PD-L1-IR700 conjugate (anti-PD-L1 conjugate) without illumination, bi-weekly doses of naked anti-PD-L1 antibody (naked anti-PD-L1), or saline (control) (FIG. 14B).

### DETAILED DESCRIPTION

Provided herein are compositions, combinations, methods and uses for treating a subject having a tumor, a lesion or a cancer, for example by activating an immune response. In some aspects, the provided embodiments involve administering to the subject a conjugate that contains a targeting molecule that binds programmed death-ligand 1 (PD-L1), conjugated with phthalocyanine dye, such as IR700. In some aspects, the provided embodiments involve illumination of a target area, such as a target area, such as a target area where cells expressing PD-L1 are or may be present. In some aspects, the illumination results in death of cells expressing PD-L1 on the surface. In some of any embodiments, the provided conjugates, compositions, combinations, methods and uses are employed for treating a subject having a tumor, a lesion (e.g., a cancerous lesion) or a cancer that has a low responsiveness or are substantially non-responsive to, has failed, has relapsed after, is refractory to and/or is resistant to prior therapeutic treatments, such as prior immunomodulatory agent treatments and/or prior anti-cancer therapeutic treatments.

In some aspects, the phthalocyanine dye-targeting molecule conjugate (e.g., anti-PD-L1 conjugated to IR700), and, in some cases, an additional therapeutic agent, are employed in the provided compositions, combinations, methods and uses. Uses include uses of the conjugates, compositions and combinations in such methods, such as therapeutic methods, and treatments, such as a treatment regimen, and uses of such conjugates, compositions and combinations in the preparation of a medicament, in order to carry out such therapeutic methods and treatments. Also provided are such conjugates, compositions and combinations for use in treating a tumor, a lesion or a cancer. In some aspect, such uses include performing the methods or treatments as described herein, such as any therapeutic methods or treatment regimens. In some embodiments, the methods and uses also involve illuminating a target area, such as a target area where the tumor, lesion or cancer is located in a subject, with a light, for example, as described herein. In some embodiments, the methods and uses thereby treat the tumor, lesion or cancer. In some aspects, the tumor, lesion or cancer to be treated include such as cancers that include primary tumors and secondary or metastatic tumor cells, for example, secondary or metastatic cancers, in a subject. In some aspects, the tumor, lesion or cancer can include a primary tumor or multiple primary tumors as well as metastatic tumor cells. In some cases, a treated subject may have one or more of primary tumors, metastatic tumor cells and/or invasive tumor cells.

In some aspects, also provided are methods and uses of such conjugates, compositions and combinations in enhancing, activating, inducing, provoking, augmenting, or supporting immune function, such as local and/or systemic immunity, in the subject. In some aspects, the provided embodiments can target cells in the tumor microenvironment, including non-cancerous cells and/or immune cells, such as immune cells that have an immunosuppressive function.

One of the great challenges in treating cancer patients is the lack of responsiveness of cancers to therapeutics. Compositions and methods for treating such cancers are urgently needed. The provided embodiments, in some contexts, are based on the observation that, treatment with a phthalocyanine dye-targeting molecule conjugate, such as a conjugate containing a PD-L1 targeting molecule and a phthalocyanine dye (e.g., IR700), followed by light illumination (also referred to as “photoimmunotherapy” and “PIT”) of a target area, results in a substantial inhibition of tumor growth and/or a complete response to the treatment.

In some aspects, treatment with a phthalocyanine dye-PD-L1 targeting molecule conjugate and light illumination can activate, induce, enhance or augment immune responses, for example, by virtue of eliminating immunosuppressive cells, such as immunosuppressive myeloid cells (e.g. myeloid-derived suppressor cells (MDSCs), tolerogenic dendritic cells (tDCs), M2 tumor associated macrophages (M2 TAMs)). In some aspects, the elimination of immunosuppressive cells results in activation, induction, enhancement or augmentation of immune responses, such as anti-tumor or anti-cancer immune responses. In some aspects, the provided embodiments offer an advantage that they can be applied to many different tumor, lesion or cancer types, e.g., cancer types of different origin or expressing different surface antigens, or cancers can share similar immunosuppressive mechanisms. In some aspects, the provided embodiments can be employed to overcome such immunosuppressive mechanisms. In addition, in some aspects, the provided embodiments can offer effective treatment of a tumor, lesion or cancer that is heterogeneous, e.g., containing various different types of tumor or cancer cells. In some aspects, the provided embodiments also offer an advantage of inducing, activating or enhancing local and/or systemic immune activity or systemic immunity in the subject, permitting treatment of tumors, lesions or cancers that are present elsewhere in the body other than the target area for illumination, such as metastasized tumor or cancer, invasive tumor or cancer, a tumor or a cancer at a different site, or a tumor, lesion or cancer of a different type. Other advantages include the treatment of metastatic cancers and/or invasive cancers without the need to locate and/or directly illuminate the metastatic tumor cells.

The provided embodiments can be also used to treat tumors, lesions or cancers that are not responsive to prior therapeutic treatments, for example, an immune checkpoint inhibitor, an anticancer agent, or a molecule against immune suppressor cells, including anti-PD-L1 immunotherapy. The provided embodiments also offer other advantages in treating cancers, such as effective treatment of cancers that are not responsive to prior therapeutic treatments, including anti-PD-L1 treatments.

The disclosure also provides unexpected features in enhancing the anti-cancer or anti-tumor immunity in a subject, for example, against a different tumor or cancer that may arise. The provided embodiments, in some contexts, are based on the observation that treatment of a cancer with a phthalocyanine dye-targeting molecule conjugate, such as an anti-PD-L1 antibody-1R700 conjugate, followed by illumination of a tumor, results in not only treatment of that particular tumor, but also results in effective treatment of a later-arising tumor of the same or a different type. The provided embodiments also offer an effective treatment of a tumor that is introduced after the subject has a complete response following the treatment of the initial tumor, indicating an immune memory response; and/or an effective treatment for a tumor that is distal to the target area for illumination (e.g., metastasized tumor or a tumor present in a different location). The provided compositions, combinations, methods and uses can result in enhancement or improvement of the subject's immune response, e.g. systemic immune response against a cancer including immune memory response, that can be effective against tumors that may develop after the treatment.

In some aspects, also provided are methods that involve the administration of an additional therapeutic agent, such as an immunomodulatory agent, in combination with the phthalocyanine dye-targeting molecule conjugate (e.g., anti-PD-L1-IR700 conjugate).

In some of any of the provided embodiments, treatment with or administration of an anti-PD-L1 conjugate is generally followed by illumination with a suitable wavelength of light. Such illumination is considered a part of the treatments and administrations of an anti-PD-L1 conjugate unless specifically stated that an illumination step is not performed with the method. In some cases, such illumination is referred to as photoimmunotherapy (PIT).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHODS OF TREATMENT WITH AND USES OF ANTI-PD-L1 CONJUGATES

In some embodiments, the provided methods and uses involve administering an anti-PD-L1 conjugate and illumination of a target area, with a wavelength of light suitable for use with the phthalocyanine dye, such that the light excites the dye and results in killing of a cell that expresses PD-L1 on its surface, for example, as described herein. Such methods and uses result in enhancing, activating, inducing, provoking, augmenting, or supporting immune function, such as local and/or systemic immunity, reducing or eliminating a lesion (e.g., tumor), reducing or inhibiting tumor growth, reducing, inhibiting, or eliminating tumor cell metastasis, or any combination thereof.

Programmed death-ligand 1 (PD-L1), also known as cluster of differentiation 247 (CD247) or B7-H1, is a protein receptor that functions as an immune checkpoint and downregulates immune responses. PD-L1 is a ligand for the immune checkpoint protein programmed cell death 1 (PD-1), expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll, 2012, Nature Reviews Cancer 12:252-264). PD-L1 is expressed on activated T cells, B cells, myeloid cells, macrophages, and certain types of tumor cells. PD-L1 is expressed on certain immune cells, such as monocytes, macrophages, dendritic cells (DC), M2 tumor associated macrophages (M2 TAM), tolerogenic dendritic cells (tDCs) or myeloid derived suppressor cells (MDSCs), or certain tumor cells, to induce immunosuppression near the tumor or the tumor microenvironment (TME).

The complex of PD-1 and PD-L1 inhibits proliferation of CD8+ T cells and reduces the immune response (Topalian et al., 2012, N Engl J Med 366:2443-54; Brahmer et al., 2012, N Engl J Med 366:2455-65). The major role of PD-1 is to limit the activity of T cells in peripheral tissues during inflammation in response to infection, as well as to limit autoimmunity (Pardoll, 2012, Nature Reviews Cancer 12:252-264). PD-1 expression is induced in activated T cells and binding of PD-1 to one of its endogenous ligands, such as PD-L1, acts to inhibit T-cell activation by inhibiting stimulatory kinases (Pardoll, 2012, Nature Reviews Cancer 12:252-264). PD-1 also acts to inhibit the TCR “stop signal” (Pardoll, 2012, Nature Reviews Cancer 12:252-264). PD-1 is highly expressed on regulatory T (Treg) cells and may increase their proliferation in the presence of ligand (Pardoll, 2012, Nature Reviews Cancer 12:252-264). The binding of PD-L1 to PD-1 transmits an inhibitory signal based on interaction with phosphatases (SHP-1 or SHP-2) via an immunoreceptor tyrosine-based switch motif (ITSM).

Anti-PD-L1 antibodies have been used for treatment of cancers, such as non-small cell lung cancer, melanoma, colorectal cancer, renal-cell cancer, pancreatic cancer, gastric cancer, ovarian cancer, breast cancer, and hematologic malignancies (Brahmer et al., N Engl J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51). In some aspects, use of anti-PD-L1 antibodies can reduce some of the immunosuppressive effect of PD-1/PD-L1, by blocking the binding of PD-L1 to PD-1.

In some aspects, however, the provided compositions, methods and uses can further enhance, activate, induce, provoke, augment, or support immune function, such as local and/or systemic immunity, by killing and eliminating cells that express PD-L1 on the surface of the cell, following illumination of a target area with a wavelength of light suitable for use with the phthalocyanine dye, such that the light excites the dye and results in killing of the cell. Thus, elimination or killing of cells expressing PD-L1 on the surface, particularly immune cells with an immunosuppressive function such as M2 tumor associated macrophages (M2 TAM), tolerogenic dendritic cells (tDCs) or myeloid derived suppressor cells (MDSCs), can function to enhance, activate, induce, provoke, augment, or support immune function, such as local and/or systemic immunity, such as anti-tumor or anti-cancer immunity. In some aspects the provided methods and uses can enhance, activate, induce, recruit, or support infiltration of lymphocytes into the tumor or lesion. In some embodiments, the provided methods and uses activate the intratumoral innate response, resulting increased activation of intratumoral dendritic cells (e.g., activated dendritic cells). In some aspects, the provided methods and uses activate the adaptive immune response, resulting in increased infiltration of CD8+ T cells. In some embodiments, the provided methods and uses lead to a reduction in the number of exhausted CD8+ T cells within the tumor. In some embodiments, the provided methods and uses lead to increased intratumoral infiltration of newly primed CD8+ T cells. In some embodiments, the provided methods and uses can result in improved therapeutic effect, such as by selecting subjects for treatment that has a higher level or number of non-exhausted effector cells, e.g., CD8+ T cells, and/or by improving the activity or response of non-exhausted effector cells.

In some aspects, the provided compositions, methods and uses can be applied to tumors resistant or refractory to anti-PD-L1 immunotherapy. Tumors, such as solid tumors, can develop resistance to anti-PD-L1 therapies by several mechanisms including, but not limited to, irreversible T cell exhaustion, insufficient T cell priming, tumor cell immunoediting leading to the upregulation of compensatory inhibitory signaling of T cells, creating an immune suppressive tumor microenvironment, such as by increasing infiltration of Tregs, MDSCs, tumor associated macrophages (e.g., M2 macrophages), elevated levels of tumor-derived cytokines and chemokines (e.g., TGF-β, CXCL8), silencing Thl type chemokines, indoleamine 2,3-dioxygenase (IDO) production, and excessive extracellular adenosine. The provided compositions, methods and uses find use in overcoming one or more anti-PD-L1 resistance mechanisms employed by some tumors.

In addition, administration of the conjugates followed by illumination can also result in direct killing of cancer cells expressing PD-L1, thereby resulting in inhibition or reduction of tumor growth. The PD-L1 targeting molecule-phthalocyanine conjugates can affect and kill, directly or indirectly, a tumor cell or cells present in a tumor or the microenvironment of a tumor (also referred to as tumor microenvironment; TME), including tumor cells at a different location than the primary tumor, metastasized tumors, newly arising tumor cells and/or tumors of different types or cell surface antigen expression. Thus, the provided compositions, methods and uses can provide an effective treatment even for tumor cells that do not express cell surface PD-L1, tumors, lesions or cancers that has a low responsiveness or are substantially non-responsive to, has failed, has relapsed after, is refractory to and/or is resistant to prior therapies, such as prior immunomodulatory agent therapies. In particular embodiments, the provided compositions, methods, and uses can treat tumors or lesions that are non-responsive, resistant, or refractory to anti-PD-L1, anti-PD-1, and/or anti-CTLA-4 therapy.

In some embodiments, the compositions, methods and uses provided herein are also effective for treating tumors that are larger in size, and which exhibit greater immune suppression than smaller tumors. Such tumors may be less responsive or non-responsive to other treatments, such as to treatment with an immunomodulatory agent, such as an immune checkpoint inhibitor (e.g., anti-PD-L1, anti-PD-1, and/or anti-CTLA-4 therapy). In such cases, anti-PD-L1 photoimmunotherapy, rendered by the administration of the anti-PD-L1 conjugate described herein followed by illumination, can be effective in inhibiting or substantially reducing growth of larger tumors that, in some cases are not effectively inhibited by other immunomodulatory agent therapies and/or anti-cancer therapies. In some embodiments, the compositions, methods and uses provided herein are effective for treating tumors that are larger in size and resistant to anti-PD-L1, anti-PD-1, and/or anti-CTLA-4 therapy.

In some aspects, provided are methods and uses of treating a tumor or a lesion in a subject by activating an immune cell response. The immune cell activation can be direct or indirect activation. In some aspects, provided are methods and uses of treating a subject having a low response to, was unresponsive to, was resistant to, was refractory to, had failed to respond to or has relapsed after, a prior immunotherapy (e.g., an anti-PD-L1, anti-PD-1, and/or anti-CTLA-4 therapy) for a tumor or a lesion. In some aspects, the methods involve administering to a subject having a tumor or a lesion a conjugate comprising a phthalocyanine dye, such as IR700, linked to a targeting molecule that binds to PD-L1, such as an anti-PD-L1 antibody. In some aspects, the methods also involve illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing cell, e.g., PD-L1 expressing immune cell, is located, such that the method can lead to the killing of the PD-L1 expressing cell and thereby inhibits the growth of the tumor or the lesion. In some aspects, the methods can lead to the killing of a PD-L1 expressing cell and thereby increases the number or activity of immune cells in the tumor or lesion and/or in the microenvironment of the tumor or lesion.

In some embodiments, provided are methods and uses of treating a tumor or a lesion in a subject by activating an immune cell response that includes illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, in a subject having a tumor or a lesion that had been administered a conjugate comprising a phthalocyanine dye linked to a targeting molecule that binds to PD-L1; wherein the method leads to the killing of the PD-L1 expressing cell and thereby inhibits the growth of the tumor or the lesion. In some embodiments, the PD-L1 expressing cell is an immune cell. In some embodiments, the PD-L1 expressing cell is a tumor cell.

In some embodiments, provided are methods and uses of treating a subject having a low response to, was unresponsive to, was resistant to, was refractory to, had failed to respond to or has relapsed after, a prior immunotherapy for a tumor or a lesion comprising illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, in a subject having a tumor or a lesion that has had a low response or that was unresponsive to a prior immunotherapy that had been administered a conjugate comprising a phthalocyanine dye linked to a targeting molecule that binds to PD-L1; wherein the method leads to the killing of a PD-L1 expressing cell and thereby increases the number or activity of immune cells in the tumor and/or in the tumor microenvironment. In some of any embodiments, the PD-L1 expressing cell is an immune cell.

In some embodiments, provided are methods and uses of enhancing a response to an anti-cancer agent in a subject having a tumor or a lesion. In some aspects, the methods involve administering an anti-cancer agent to a subject having a tumor or a lesion. In some aspects, administering a conjugate comprising a phthalocyanine dye linked to a targeting molecule that binds to PD-L1 to the subject. In some aspects, illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, leading to a greater inhibition of growth of the tumor or the lesion compared to the inhibition by treatment with the anti-cancer agent alone.

In some embodiments, provided are methods and uses of enhancing a response to an anti-cancer agent in a subject having a tumor or a lesion that involves: administering to a subject a conjugate comprising a phthalocyanine dye linked to a targeting molecule that binds to PD-L1 to the subject; and illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, wherein the subject had been administered an anti-cancer agent; leading to a greater inhibition of growth of the tumor or the lesion compared to the inhibition by treatment with the anti-cancer agent alone.

In some embodiments, provided are methods and uses of enhancing a response to an anti-cancer agent in a subject having a tumor or a lesion involves: administering an anti-cancer agent to a subject; wherein the subject had received a treatment comprising administering a conjugate comprising a phthalocyanine dye linked to a targeting molecule that binds to PD-L1 to the subject; and illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, and wherein the administration of the anti-cancer agent and the treatment leads to a greater inhibition of growth of the tumor or the lesion compared to the inhibition with the anti-cancer agent alone.

In some embodiments, provided are methods and uses of vaccinating or immunizing a subject to generate an anti-cancer immune response. In some aspects, vaccinating or immunizing a subject to generate an anti-cancer immune response can inhibit the growth and/or reduce the size of a first tumor or lesion; and also delay or prevent the appearance, growth or establishment of one or more second tumors or lesions, for example located distally to the treated first tumor or lesion. In some aspects, the methods involve administering a conjugate comprising a phthalocyanine dye linked to a targeting molecule that binds to PD-L1 to a subject. In some aspects, the methods involve illuminating a target area, leading to an anti-cancer response selected from a delay or inhibition in the appearance of or growth of a tumor in the subject or an appearance or increase in T memory cells in the vicinity of a tumor.

In some embodiments, provided are methods and uses of vaccinating or immunizing a subject to generate an anti-cancer immune response that involves: illuminating a target area in a subject that had been administered a conjugate comprising a phthalocyanine dye linked to a targeting molecule that binds to PD-L1; leading to an anti-cancer response selected from a delay or inhibition in the appearance of or growth of a tumor in the subject or an appearance or increase in T memory cells in the vicinity of a tumor.

In some embodiments, one or more of steps of the method are repeated. In some embodiments, the administration of the conjugate is repeated one or more times, optionally wherein after each repeated administration of the conjugate, the illuminating step is repeated. In some embodiments, further comprising administering an additional therapeutic agent or anti-cancer treatment.

A. Methods for Stimulating or Enhancing Anti-Cancer Immune Responses

In some aspects, the provided methods and uses employing compositions including an anti-PD-L1 conjugate can result in an enhancement of an immune response, such as systemic and/or local immune response in the subject, which in turn can result in an enhanced response to the therapy or treatment for a tumor, a lesion or a cancer. In some aspects, the methods and uses herein include administering to the subject anti-PD-L1 conjugate, and after administration of the conjugate, illuminating the target area, such as a target area where PD-L1-expressing cells are present, e.g., a tumor, the vicinity of a tumor, a lymph node, the vicinity of the lymph node, or the tumor microenvironment.

In some aspects, the provided embodiments can stimulate, enhance, activate, induce, provoke, boost, augment, or support an immune response, such as a systemic immune response, in a subject having a tumor, a lesion or a cancer. In some embodiments, the provided method and uses results in enhancing a systemic immune response in a subject having a tumor, a lesion or a cancer. “Systemic immune response” refers to the ability of a subject's immune system to respond to an immunologic challenge or immunologic challenges, including those associated with a tumor, a lesion or a cancer, in a systemic manner. Systemic immune response can include systemic response of the subject's adaptive immune system and/or innate immune system. Systemic immune response can include anti-tumor or anti-cancer response from the subject's adaptive immune system and/or innate immune system. In some aspects, systemic immune response includes an immune response across different tissues, including the blood stream, lymph node, bone marrow, spleen and/or the tumor microenvironment, and in some cases, includes a coordinated response among the tissues and organs and various cells and factors of the tissues and organs. In some embodiments, the provided embodiments can stimulate, enhance, activate, induce, provoke, boost, augment, or support the anti-cancer or anti-tumor immune response of the subject's own immune system, including the adaptive immune system and/or innate immune system. In some aspects, the provided methods and uses can result in enhancement of an innate immune response in the subject.

In some aspects, the provided embodiments can also exhibit an abscopal effect. In some aspects, “abscopal effect” refers to a treatment effect in which a tumor that is not directly treated or is away from the site of localized treatment, e.g., a distal or a metastatic tumor, is also treated, for example, reduced in tumor volume.

In some aspects, the provided embodiments can effect tumor immunity. In such aspects, the provided embodiments prevent or impede growth of a new tumor or a metastasis. In some embodiments, inhibition of tumor growth effected by the provided embodiments leads to a durable anti-tumor response. In some embodiments, inhibition of tumor growth effected by the provided embodiments leads to prolonged progression-free survival. In some embodiments, inhibition of tumor growth effected by the provided embodiments leads to a reduced chance of relapse and/or a reduced chance of metastasis. In some aspects, the provided embodiments can effect immunity for the same tumor type or a different tumor type in the treated subject. In some aspects, the provided embodiments can inhibit growth of tumors from a different tumor lineage, i.e., a different type of tumor that arises or could arise in a treated subject.

In some aspects, the target area is an area that comprises PD-L1 expressing cells. In some embodiments, the PD-L1 expressing cell is an immune cell. In some of any embodiments, the methods lead to the killing of the PD-L1 expressing cells, such as PD-L1 expressing immune cells. In some embodiments, the PD-L1 expressing immune cell is selected from the group consisting of monocytes, macrophages, dendritic cells (DC), M2 tumor associated macrophages (M2 TAM), tolerogenic dendritic cells (tDC) and myeloid derived suppressor cells (MDSC). In some embodiments, the PD-L1 expressing immune cell is located in the tumor, the tumor microenvironment or a lymph node.

In some aspects, the target area to be illuminated in accordance with the provided embodiments is a tumor, such as a primary tumor, the vicinity of a tumor, such as a primary tumor, or the tumor microenvironment (TME). In some embodiments, the target area is near the tumor or proximal to the tumor, or the vicinity of the tumor or tumor cells. In some embodiments, the target area is a tumor. In some embodiments, the target area is a primary tumor. In some embodiments, the target area is a secondary tumor or a metastasized tumor. In some embodiments, the target area is the tumor microenvironment.

In some embodiments, the target area is a lymph node or the vicinity of the lymph node. In some embodiments, the target area is a lymph node, for example, containing PD-L1 expressing cells, or the vicinity of the lymph node. In some embodiments, the target area is a lymph node. In some embodiments, the target area is a vicinity of a lymph node.

In some aspects, the provided embodiments can stimulate or enhance a systemic response, such as a systemic immune response, against one or more primary tumors or lesions and/or one or more second tumors or lesions, such as metastatic tumors or lesions, or tumors or lesions of a different type.

In some aspects, the provided embodiments stimulate or enhance the subject's immune response, such as the subject's anti-cancer immune response, in some cases by removing PD-L1 expressing immune cells, such as those that may have an immunosuppressive function, such as monocytes, macrophages, dendritic cells (DC), including M2 tumor associated macrophages (M2 TAM), tolerogenic dendritic cells (tDCs), or myeloid derived suppressor cells (MDSCs). In some aspects, the provided embodiments stimulate or enhance the subject's immune response, such as the systemic and/or local immune response that target the tumor, lesion or cancer, by killing and eliminating PD-L1 expressing immunosuppressive cells, such as M2 TAM, tDCs or MDSCs. As exemplified herein, inhibition of tumor growth following administration of an PD-L1-phthalocyanine dye conjugate and light illumination requires the presence and/or activity of the subject's CD8+ T cells, as depletion of the subject's CD8+ T cells resulted in tumor growth similar to the growth in saline administered control. In some aspects, the immunosuppressive cells, e.g., M2 TAM, tDCs or MDSCs, such as those that express PD-L1, inhibit the function and/or activity of the subject's immune cells, such as CD8+ T cells or natural killer (NK) cells. By virtue of killing and eliminating the immunosuppressive cells, such as PD-L1 expressing immunosuppressive cells, including M2 TAM, tDCs or MDSCs, the provided embodiments can stimulate and enhance the subject's immune response. As exemplified herein, such treatment according to the provided embodiments results in inhibition of growth, such as a complete response to the treatment, of one or more primary tumors, and inhibition of growth of one or more second tumor, such as a second tumor of the same or different types and/or origin and/or a second tumor present at a different site, such as a distal site, from the primary tumor or lesion.

In some aspects, inhibition of the growth of the tumor or the lesion and/or killing of the PD-L1 expressing cell is dependent on the presence of CD8+ T cells. In some embodiments, prior to the administering, the subject has a tumor or a lesion having a low number or level of CD8+ T cell infiltration. In some embodiments, the number, level or activity of immune cells is increased in the tumor or in the tumor microenvironment after the administering and the illuminating. In some embodiments, the number or level of CD8+ T cell infiltration in the tumor or the lesion is increased after the administering and the illuminating. In some embodiments, the number of memory T cells in the vicinity of the tumor is increased after the administering and the illuminating.

In some aspects, the stimulated or enhanced systemic immune response includes an increase in the number and/or activity of systemic CD8⁺ T effector cells, an increase in systemic T cell cytotoxicity against tumor cells as measured using a CTL assay using cells from the spleen, the peripheral blood, the bone marrow, or the lymph nodes, an increase in the number, activity and/or priming of intratumoral CD8⁺ T effector cells in the primary or secondary (e.g., metastatic or new) tumors or lesions, an increase in systemic CD8⁺ T cell activation, an increase in systemic dendritic cell activation, an increase in dendritic cell activation in the primary or secondary (e.g., metastatic or new) tumors or lesions, an increase in intratumoral dendritic cell infiltration in the primary or secondary (e.g., metastatic or new) tumors or lesions, an increase in new T cell priming in the primary or secondary (e.g., metastatic or new) tumors or lesions, an increase in T cell diversity in the primary or secondary (e.g., metastatic or new) tumors or lesions, a decrease in systemic regulatory T cells, a decrease in regulatory T cells in the primary or secondary (e.g., metastatic or new) tumors or lesions, a decrease in systemic myeloid derived suppressor cells, a decrease in intratumoral myeloid derived suppressor cells in the primary or secondary (e.g., metastatic or new) tumors or lesions, a decrease in tumor associated fibroblasts or cancer associated fibroblasts (CAFs), in the primary or secondary (e.g., metastatic or new) tumors or lesions, or any combination thereof in the subject. In some instances, a systemic response can be assessed by sampling blood, tissue, cells or other fluid from a subject and assessing an increase in pro-inflammatory cytokines, an increase or appearance of immune cell activation markers and/or T cell diversity. In some aspects, a systemic response may be assessed by assaying cells affected directly or indirectly by the methods. For example, cell can be collected from the subject between day 4 and day 28 after treatment or any time after the step of illumination of the primary tumor in the subject.

In some aspects, the provided embodiments can stimulate, enhance, boost, augment, or support an immune response, such as a local response, such as a local immune response, in a subject having a tumor, a lesion or a cancer. In some embodiments, the provided method and uses results in enhancing a local response in a subject having a tumor, a lesion or a cancer. “Local immune response” refers to the immune response in a tissue or an organ to an immunologic challenge or immunologic challenges including those associated with a tumor, a lesion or a cancer. A local immune response can include the adaptive immune system and/or innate immune system. In some aspects, local immunity includes immune response concurrently occurring at different tissues, such as the blood stream, lymph node, bone marrow, spleen and/or the tumor microenvironment.

In some aspects, the stimulated or enhanced local immune response includes an increase in the number and/or activity of intratumoral CD8⁺ T effector cells (e.g., CD3⁺ CD8⁺ cells), an increase in CD8⁺ T effector cell activation, an increase in intratumoral dendritic (CD11c⁺) cell infiltration, an increase in intratumoral dendritic cell activation (e.g., CD11c⁺ CD80⁺ and/or CD11c⁺ CD40⁺), an increase in intratumoral antigen-presenting dendritic cells (CD11b⁺ CD103⁺ CD11c⁺), an increase in intratumoral new T cell priming (e.g., CD3⁺ CD8⁺ PD1⁻ cells), an increase in intratumoral T cell diversity, an increase in intratumoral neutrophils (CD11b⁺ Cy6C^{-/low} Ly6G⁺ cells), a decrease in intratumoral macrophages (e.g., CD11b⁺ F4/80⁺ cells), a decrease in intratumoral regulatory T cells (Tregs), a decrease in intratumoral myeloid derived suppressor cells (MDSCs; e.g., CD11b⁺ Ly6C⁺ Ly6G⁺ cells), a decrease in intratumoral tumor associated fibroblasts or cancer associated fibroblasts (CAFs), a decrease in the number and/or activity of intratumoral exhausted T cells, such as exhausted CD8+ T cells (e.g., PD-1⁺CTLA-4⁺CD3⁺CD8⁺ cells), or any combination thereof in the subject. In some aspects, the stimulated or enhanced local immune response is effected by any of the provided embodiments. In some aspects, the cell surface phenotype of cells, such as immune cells indicative of local immune response or innate immune response, is assessed by staining with reagents, such as labelled antibodies, that can be used to detect the expression of the marker(s) on the surface. In some aspects, the cell surface phenotype of cells, such as immune cells indicative of local immune response or innate immune response, is detected using flow cytometry.

In some cases, a local response, such as a local immune response, can be assessed by taking a blood, tissue or other sample from a subject and assessing for an increase in an anti-immune cell type in the tumor or TME and/or assessing for an increase or appearance of local immune activation markers. In some aspects, a local response, such as a local immune response, may be assessed by assaying cells affected directly or indirectly by the methods. For example, cell can be collected from the subject between day 4 and day 28 after treatment or any time after the step of illumination of the primary tumor in the subject.

In some aspects, the methods and uses also involve administering an additional therapeutic agent, such as an immunomodulatory agent, e.g., an immune checkpoint inhibitor. The immunomodulatory agent can be administered prior to, concurrent with or subsequent to the administration of the conjugate. In some aspects, administration of the additional therapeutic agent, such as an immunomodulatory agent, can also contribute to stimulating, enhancing, activating, inducing, augmenting or supporting an immune response, such as the subject's systemic and/or local immune response, including anti-cancer or anti-tumor responses. Exemplary additional therapeutic agents, compositions, combinations, methods and uses include those described herein, e.g., in Section V.

B. Tumors and Lesions for Anti-PD-L1 Conjugate Therapy

The methods described herein include administration of an anti-PD-L1 conjugate and illuminating a target area, such as a tumor or a lesion, the vicinity of a tumor, a lymph node, the vicinity of a lymph node, or the tumor microenvironment (TME) of a tumor or a lesion, in a subject with a wavelength of light to activate the phthalocyanine dye moiety of the conjugate to achieve cell killing, for example, of cells expressing PD-L1 on the surface. In some embodiments, the methods and uses provided herein include treating a subject that has one or more tumors or lesions, such as one or more primary tumors or lesions (or first tumors or lesions), one or more secondary tumors or lesions (or second tumors or lesions), one or more newly arising tumors or lesions and/or one or more metastasized tumors or lesions. The subject may have one, two, three, or more than three tumors. Such tumors can be in one or more tissues or organs, such as in one tissue or organ, in two different tissues or organs, in three different tissues or organs, or in more than three different tissues or organs. In some aspects one or more of the tumors to be treated expresses PD-L1 on the surface of the cells of which the tumor is comprised. In some aspects, one or more of the tumors to be treated contain, are primarily composed of, have a substantial number of, or are entirely composed of cells that do not express PD-L1, have low PD-L1 expression, or are PD-L1-negative. In some aspects, one or more of the tumors to be treated contain, are primarily composed of, have a substantial number of, or are entirely composed of cells that have a reduced response, are resistant to, or become resistant to (i.e., acquire resistance to) PD1/PD-L1 checkpoint blockade.

In some aspects, the tumor or lesion treated according to the provided embodiments is treatment-naïve for, or has not previously received a treatment with, an immune checkpoint inhibitor, such as naïve for anti-PD-1, anti-PD-L1 and/or anti-CTLA-4 therapy/therapies. In some embodiments, the tumor or lesion has not received (is naïve to) anti-PD-1 treatment. In some embodiments, the tumor or lesion has not received (is naïve to) anti-PD-L1 treatment. In some embodiments, the tumor or lesion has not received (is naïve to) anti-CTLA-4 treatment. In some embodiments, the subject having a tumor or lesion to be treated according to the provided embodiments is treatment-naïve for an immune checkpoint inhibitor. In some embodiments, the subject to be treated is naïve to anti-PD-1 treatment. In some embodiments, the subject to be treated is naïve to anti-PD-L1 treatment. In some embodiments, the subject to be treated is naïve to anti-CTLA-4 treatment. Treating a subject with an immune checkpoint inhibitor, such as an anti-PD-1 antibody, can lead to CD8+ effector T cell exhaustion in the tumor, its periphery, and/or systemically. This can lead to the inability of the CD8+ T cells to recognize and localize to the tumor, or it can render the CD8+ T cells ineffective despite localization to the tumor or to the vicinity of the tumor, leading to checkpoint inhibitor (e.g., PD-1/PD-L1) resistance. Hence, in some instances, ineffective or insufficient CD8+ effector T cell activity can be mitigated by avoiding immune checkpoint inhibitor therapy (e.g., anti-PD-1, anti-PD-L1 and/or anti-CTLA-4 therapy) prior to employing the provided compositions, methods, or uses. In some embodiments, the response of a tumor or lesion to treatments herein is effected by first treating the tumor or lesion with the administration of an anti-PD-L1 conjugate followed by illumination, prior to any treatment of the tumor or lesion with an immune checkpoint inhibitor, such as a PD-1, PD-L1, and/or CTLA-4 directed therapy (such as an anti-PD-1 antibody, and anti-PD-L1 antibody, and/or anti-CTLA-4 antibody). In some embodiments, methods of treatment include selecting subjects that have not received treatment with an immune checkpoint inhibitor (e.g., anti-PD-1, anti-PD-L1, and/or anti-CTLA-4) therapy and treating such subjects (i.e. a tumor or lesion of such subject) with an anti-PD-L1 conjugate followed by illumination.

In some aspects, tumor or the lesion for treatment in accordance with the provided embodiments is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

In some aspects, the tumor or lesion treated according to the provided embodiments include one or more primary (e.g., first) tumor or lesion. In some aspects, a primary tumor or lesion can include the first or original tumor or lesion in a subject. In some aspects, the subject can have one or more primary tumors or lesions. In some embodiments, a primary tumor or primary tumors may be a solid tumor or solid tumors, may be lymphomas, or may be leukemias. The tumor can be tumor of the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, or unknown primary origin.

In some aspects, the tumor or lesion treated according to the provided embodiments include one or more second tumor or lesion, such as a metastatic tumor or lesion, or a newly arising tumor or lesion. In some aspects, the one or more second tumor or lesion is derived from a metastasis of the first tumor or lesion. In some embodiments, the one or more second tumor or lesion is a tumor that is not derived from a metastasis of the first tumor or lesion. In some aspects, the one or more second tumor or lesion is phenotypically and/or genotypically different from the first tumor or lesion. In some aspects, the one or more second tumor or lesion is phenotypically different from the first tumor or lesion. In some aspects, the one or more second tumor or lesion is genotypically different from the first tumor or lesion. In some aspects, the one or more second tumor or lesion is a newly arising tumor or lesion. In some aspects, the one or more second tumor or lesion is from a different origin compared to the first tumor or lesion. In some aspects, the one or more second tumor or lesion arises from a different organ or a different cell compared to the first tumor or lesion. In some embodiments, the one or more second tumor or lesion may be a solid tumor or solid tumors, may be lymphomas, or may be leukemias. The one or more second tumor or lesion can be tumor of the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, or unknown origin.

In some embodiments, immunity to a second tumor or lesion is effected when the first tumor is illuminated, following administration of the provided anti-PD-L1 conjugate, and the volume of the first tumor is reduced. In such embodiments, the volume of the first tumor is reduced by at least or at least about 10%, 15%, 20%, 25%, 30%, 35%, 40%, 45%, 50%, 55%, 60%, 65%, 70%, 75%, 80%, 85%, 90%, 95%, or 100%. In some embodiments, the volume of the first tumor is reduced by at least or at least about 50%. In some embodiments, the volume of the first tumor is reduced by at least or at least about 75%. In some embodiments, immunity to a second tumor or lesion is effected when the first tumor achieves partial or complete response (PR or CR) following treatment of the first tumor. In some embodiments, immunity to a second tumor or lesion is effected when the first tumor achieves CR following treatment of the first tumor.

In some cases, the target area for illumination can be the primary tumor or lesion, or the vicinity of the primary tumor or lesion. In other cases, the target area for illumination is not the primary tumor or lesion, but a different area where PD-L1 expressing cells are present, such as the lymph node, or a secondary tumor or lesion or the vicinity of a secondary tumor or lesion.

In some aspects, following treatment according to the provided embodiments, with anti-PD-L1 conjugate treatments and light illumination, the growth of the one or more primary tumors or lesions is inhibited, the volume of the one or more primary tumors or lesions is reduced or both tumor growth and volume are reduced. In some aspects, following treatment according to the provided embodiments, with anti-PD-L1 conjugate treatments and light illumination, the growth of the one or more secondary or metastatic tumors or lesions is inhibited, the volume of the one or more secondary or metastatic tumors or lesions is reduced or both tumor growth and volume are reduced. In some aspects, treatment according to the provided embodiments delays regrowth of the tumor or the lesion, prevents a relapse of a cancer or prolongs the duration of remission of a cancer, such as a cancer associated with the tumor or lesion, prevents or inhibits the generation and/or growth of one or more second tumor or lesion, including a second tumor or lesion of a different type compared to the primary tumor or lesion, and/or prevents or inhibits the generation and/or growth of a metastasis.

In some embodiments, the subject is administered the anti-PD-L1 conjugate to treat and/or inhibit the growth of a first tumor or a first lesion; and the method inhibits or delays the appearance of one or more second tumors or lesions or a metastasis of the first tumor or the first lesion.

In some aspects, the primary tumor or lesion contains cells that express PD-L1 on the surface. In some aspects, the cells that express PD-L1 is an immune cell, such as an immunosuppressive cell, e.g., M2 TAM, tDCs or MDSCs. In some aspects, the PD-L1 expressing cell is a tumor associated fibroblast or a cancer associated fibroblasts (CAF). In some cases, the cells that express PD-L1 is a tumor cell or a cancer cell. In some of any embodiments, the subject to be treated has one or more of the PD-L1 expressing cells, such as one or more PD-L1 expressing cells that are associated with the tumor, lesion or cancer.

In some embodiments, the tumor, lesion or cancer to be treated is contains tumor or cancer cells that do not express PD-L1. In some embodiments, the tumor or the lesion comprises PD-L1 negative tumor cells. In some embodiments, more than at or about 40%, 50%, 60%, 70%, 80%, 90% or 95% of the tumor cells in the tumor or the lesion are PD-L1 negative tumor cells. In some aspects, PD-L1 negative tumor cells can refer to tumor cells that do not express detectable levels of PD-L1 on its surface or tumor cells that express PD-L1 at a level less than a threshold level, such as a detectable threshold level. In some embodiments, a PD-L1 negative tumor cell includes a tumor cell is not specifically recognized by an anti-PD-L1 antibody. In some cases, the level of PD-L1 expression is determined by flow cytometry. In some aspects, the provided embodiments result in indirect killing of tumor cells or cancer cells, such as by eliminating immunosuppressive cells, e.g., M2 TAM, tDCs or MDSCs, and enhancing the function and/or activity of effector cells in the immune system, such as CD8+ T cells, which can exert an anti-tumor or anti-cancer response to eliminate the tumor cells or cancer cells.

In some embodiments, the tumor, lesion or cancer to be treated is contains tumor or cancer cells that express PD-L1. In some aspects, administration of the anti-PD-L1 conjugate followed by light illumination can directly kill cells that express PD-L1. In some aspects, the provided embodiments result in a direct killing of PD-L1-expressing tumor cells.

In some embodiments, the methods and uses provided herein include treating a subject that has invasive tumor cells, such as when cells originating from a primary tumor have invaded into surrounding tissues. The methods include administering to a subject having invasive tumor cells, an anti-PD-L1 conjugate and after administration of the conjugate, illuminating the target area with a wavelength suitable for the selected phthalocyanine dye. In some embodiments, the methods include the administration of an immunomodulatory agents, such as an immune checkpoint inhibitor prior to, concurrent with, or subsequent to the administration of the conjugate. In some aspects, invasive tumor cells refer to cells originated from a primary tumor and have invaded into surrounding tissues of the same organ or neighboring organs or body cavities of the primary tumor within the body of a subject having the primary tumor.

In some instances, the methods and uses provided herein include illumination of a target area. In some aspects, the target area the one or more primary tumors, and some or all of the invasive tumor cells are not illuminated, and in such methods, the growth of invasive tumor cells is inhibited, reduced or eliminated, the volume of one or more invasive tumors is reduced or any combination thereof. In some embodiments, the growth of the primary tumor also is inhibited, reduced or eliminated, the volume of one or more primary tumors also is reduced along with the effect(s) on the one or more invasive tumor cells.

In some embodiments, invasive tumor cells are contained in a solid tumor. In some embodiments, invasive tumor cells are contained in body fluids, including but not limited to peritoneal fluid, pleural fluid, and cerebrospinal fluid. In some embodiments, invasive tumor cells are contained in the effusion of a body cavity or body cavities, including but not limited to peritoneal effusion (ascites), pleural effusion, and pericardial effusion.

In some embodiments, the methods and uses provided herein include treating a subject that has one or more primary tumors and also metastatic tumor cells. The methods include administering to a subject having primary tumor(s) and metastatic tumor cells, an anti-PD-L1 conjugate and after administration of the conjugate, illuminating the target area with a wavelength suitable for the selected phthalocyanine dye. In such methods, the growth of metastatic tumor cells is inhibited, reduced or eliminated, the volume of one or more metastatic tumors is reduced or any combination thereof.

In some embodiments of the methods and uses provided herein, metastatic tumor cells are distal to the primary tumor and some or all of the metastatic tumor cells are not illuminated, e.g., not directly illuminated.

In some embodiments of the methods and uses, only a target area, such as a target area containing and/or is in the vicinity of a lymph node or a primary tumor or lesion, is illuminated. In some aspects, the second tumor or lesion, such as a metastatic tumor or lesion, is not illuminated.

In some aspects, metastatic tumor cells include cells originated from a primary tumor and spread to distal tissue or organ, or distal tissues or organs within the body of a subject having the primary tumor. The metastatic tumor cells can be located in one or more locations in the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, blood, bone marrow, cerebrospinal fluid, or any other tissues or organs. In some embodiments, metastatic tumor cells are contained in a solid tumor. In some embodiments, metastatic tumor cells are circulating tumor cells or are not associated with a tumor mass.

In some embodiments, the methods and uses include the administration of an immunomodulatory agent, such as a checkpoint inhibitor prior to, concurrent with or subsequent to the administration of the conjugate. In some embodiments, the methods and uses include the administration of a second conjugate, such as a second immunoconjugate, followed by illumination, concurrent with, prior to, or subsequent to the administration of the instantly provided conjugate. In some embodiments, the methods and uses include the administration of one or more additional anti-cancer treatments, such one or more of a chemotherapy, antiangiogenic therapy, a kinase inhibitor, a radiotherapy, small molecule therapy or other treatment, such as any treatment described in the Section entitled “Combination Therapy” herein.

C. Methods and Compositions for Treating a Tumor or Tumor Cells that are Less Responsive to, Refractory to, or Not Responsive to Prior Therapeutic Treatments

In some embodiments, provided are compositions containing an anti-PD-L1 conjugate, i.e., a phthalocyanine dye-targeting molecule conjugate, in which the targeting molecule binds to PD-L1 (e.g., an anti-PD-L1 antibody-IR700 conjugate), and methods and uses involving the anti-PD-L1 conjugate for therapy or treatment of a tumor or a cancer that has failed, that was less responsive to, that has not achieved a desired level of response, that achieved a less than desired level of response to (e.g., poorly responsive or not therapeutically effective)or was not responsive to one or more prior treatments, such as with an immunomodulatory agent, such as an immune checkpoint inhibitor and/or an anticancer agent, such as an anti-cancer agent that directly targets tumor or cancer cells. In some embodiments, the tumor or cancer achieved a less than desired level of response or is predicted to be resistant to anti-PD-L1, anti-PD-1, and/or anti-CTLA-4 therapy. In some embodiments, the tumor or cancer achieved a less than desired level of response or is predicted to be resistant to anti-PD-L1 therapy. In some embodiments, the tumor or cancer achieved a less than desired level of response or is predicted to be resistant to anti-PD-1 therapy. In some embodiments, the tumor or cancer achieved a less than desired level of response or is predicted to be resistant to anti-CTLA-4 therapy.

The cancers include a primary tumor or multiple primary tumors as well as metastatic tumor cells, for example metastatic cancers; newly arising tumors or cancers; a cancer that includes a primary tumor or multiple primary tumors; and/or invasive tumor cells, for example, invasive cancers. In some aspects, the provided compositions, methods, uses and combinations can also sensitize cold tumors, including primary cold tumors and secondary cold tumors (e.g., metastatic tumors), to immunomodulatory agents or other anti-cancer therapy.

Such methods and uses include, for example, administration of an anti-PD-L1 conjugate to a subject having a tumor or tumor cells followed by illumination of a target area (e.g., sites where PD-L1 expressing cells are present), using a suitable light wavelength and dose for the phthalocyanine dye. In some aspects, the illumination results in an illumination-dependent lysis and death of cells expressing the target molecule (e.g., PD-L1) on the surface, resulting in a therapeutic effect or treatment of the cancer. In some cases, cells expressing PD-L1, such as monocytes, macrophages, dendritic cells (DC), M2 tumor associated macrophages (M2 TAM), tolerogenic dendritic cells (tDCs) or myeloid derived suppressor cells (MDSCs), or certain tumor cells, are killed and thus rapidly deplete. As a result, necrosis of the tumor cells can occur.

In some aspects, the tumor, lesion or cancer to be treated in accordance with the provided embodiments include those that have failed, had a low responsiveness or were substantially non-responsive to, that was less responsive to, that had not achieved a desired level of response, that achieved a less than desired level of response to (e.g., poorly responsive or not therapeutically effective), has relapsed after, were refractory to and/or were resistant to one or more prior treatments, such as with an immunomodulatory agent, e.g., an immune checkpoint inhibitor and/or an anticancer agent, such as an anti-PD-L1, anti-PD-1, or anti-CTLA-4 therapy.

In some embodiments, the subject to be treated in accordance with the provided embodiments, has been previously treated with an anti-cancer treatment and/or an immune checkpoint inhibitor. In some embodiments, the subject to be treated in accordance with the provided embodiments, has been previously treated with an immune checkpoint inhibitor. In some embodiments, the subject to be treated in accordance with the provided embodiments, has failed or has relapsed after the previous treatment with the anti-cancer treatment and/or immune checkpoint inhibitor. In some embodiments, the subject to be treated in accordance with the provided embodiments, has failed or has relapsed after the previous treatment with the immune checkpoint inhibitor.

In some embodiments, the inhibition of tumor growth resulting from carrying out the method is greater compared to the inhibition of tumor growth as a result of the previous treatment with the anti-cancer treatment and/or immune checkpoint inhibitor (e.g., anti-PD-L1, anti-PD-1, and/or anti-CTLA-4 therapy). In some embodiments, the inhibition of tumor growth resulting from carrying out the method is greater compared to the inhibition of tumor growth as a result of the previous treatment with the immune checkpoint inhibitor (e.g., anti-PD-L1, anti-PD-1, and/or anti-CTLA-4 therapy).

In some aspects, the prior therapeutic treatment or treatments to which the cancers are not responsive include using an anticancer agent. The prior anticancer agent can be one or more of: a chemotherapeutic agent, an antibody treatment, and/or a radiotherapeutic agent. In some embodiments, the prior therapy is therapy with an anti-cancer agent selected from a checkpoint inhibitor, an immune adjuvant, a chemotherapeutic agent, radiation, and a biologic comprising an anti-cancer targeting molecule that binds to a tumor cell. In some embodiments, the prior therapy is therapy with an anti-cancer agent that is an antibody conjugate. In some embodiments, the prior therapy is therapy with an antibody conjugate comprising a phthalocyanine dye, a toxin, or a TLR agonist.

In some aspects, the prior therapeutic treatment or treatments to which a cancer, tumor, or tumor cells are not responsive can be treatment with an immune checkpoint inhibitor (also known immune checkpoint blockade therapy). The prior immune checkpoint inhibitor can be a PD-1 inhibitor, a PD-L1 inhibitor, a CTLA-4 inhibitor or combination thereof. The prior immune checkpoint inhibitor can be a small molecule inhibitor, an antibody inhibitor, or other molecule that binds to and inhibits an immune checkpoint protein, such as PD-1 or PD-L1. Exemplary antibody inhibitors for PD-1 include, but are not limited to, any of pembrolizumab (MK-3475, Keytruda), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZMO09, camrelizumab (SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb20717, R07121661, CX-188, and spartalizumab. Exemplary antibody inhibitors for PD-L1 include, but are not limited to, any of atezolizumab (MPDL3280A, Tecentriq), avelumab (Bavencio), durvalumab (MEDI4736, Imfinzi), LDP, NM-01, STI-3031, KN035, LY3300054, M7824 (MSB0011359C), BMS-936559, MSB2311, BCD-135, BGB-A333, CBT-502, cosibelimab (CK-301), CS1001, FAZ053, MDX-1105, SHR-1316, TG-1501, ZKAB001, INBRX-105, MCLA-145, KN046, LY3415244, REGN3504, and HLX20.

In some aspects, the tumor, lesion or cancer to be treated include tumors or cancers that were resistant to, refractory to, or not responsive to, anti-PD-1 antibody or anti-PD-L1 antibody treatment. In some aspects, the tumor, lesion or cancer to be treated include tumors or cancers that were resistant to, refractory to, or not responsive to anti-PD-L1 antibody treatment or are predicted to be unresponsive, resistant, or refractory to anti-PD-L1 antibody treatment. In some aspects, the tumor, lesion or cancer to be treated include tumors or cancers that were resistant to, refractory to, or not responsive to anti-PD-1 antibody treatment or are predicted to be unresponsive, resistant, or refractory to anti-PD-1 antibody treatment.

In some aspects, the prior treatment is treatment with anti-CTLA-4 antibody, such as ipilimumab (YERVOY), tremelimumab, AGEN1181, AGEN1884, ADU-1064, BCD-145, and BCD-217. In some aspects, the tumor, lesion or cancer to be treated include tumors or cancers that were resistant to, refractory to, or not responsive to anti-CTLA-4 antibody treatment or are predicted to be unresponsive, resistant, or refractory to anti-CTLA-4 antibody treatment.

In some aspects, the prior therapeutic treatment or treatments to which a cancer, tumor or tumor cells are not responsive can be treatment with an immunomodulatory agent such as a cytokine, for example, Aldesleukin (PROLEUKIN), Interferon alfa-2a, Interferon alfa-2b (Intron A), Peginterferon Alfa-2b (SYLATRON/PEG-Intron), or a cytokine that targets the IFNAR1/2 pathway, the IL-2/IL-2R pathway, or such as an adjuvant, for example, Poly ICLC (HILTONOL/Imiquimod), 4-1BB (CD137; TNFRS9), OX40 (CD134) OX40-Ligand (OX4OL), Toll-Like Receptor 2 Agonist SUP3, Toll-Like Receptor TLR3 and TLR4 agonists and adjuvants targeting the Toll-like receptor 7 (TLR7) pathway, other members of the TNFR and TNF superfamilies, other TLR2 agonists, TLR3 agonists and TLR4 agonists.

In some aspects, the prior therapeutic treatment or treatments to which the cancers are not responsive include using a therapeutic agent targeted against immunosuppressive cells. The agent can be an antibody, for example, anti-CD25 antibodies, such as basiliximab (Simulect®), daclizumab or PC61, that target regulatory T cells; a small molecule inhibitor or combination thereof. Immunosuppressive cells include regulatory T cells, M2 macrophages, tumor associated fibroblasts or cancer associated fibroblasts (CAFs), or combination thereof.

In some instances, a tumor, lesion or cancer to be treated in accordance with the provided embodiments include a “cold tumor” or a “cold cancer,” such as a tumor that has an immunosuppressive phenotype. Such cold tumors can have features including, but not limited to, a substantial reduction in numbers and/or activities or absence of intratumoral CD8⁺ T effector cells and/or substantial increase in numbers and/or activities of intratumoral immune suppressor cells. In some cases, a cold tumor or cancer has a high tumor mutational burden (TMB), an immune score indicative of low immunoresponsiveness, a programmed cell death protein 1 (PD-1) or programmed death-ligand 1 (PD-L1) marker status (e.g., cell surface expression), which could be indicative of low immunoresponsiveness. In some instances, a cold tumor or cancer does not respond to PD-1 or PD-L1 inhibitor monotherapy.

In some embodiments, cold tumors or cancers can be treated with anti-PD-L1 conjugate, followed by illumination, as described herein. In some embodiments, a combination treatment with an anti-PD-L1 conjugate, followed by illumination, and an immunomodulatory agent, such as an immune checkpoint inhibitor, results in enhanced inhibitory effects on the growth of both illuminated primary tumor and a distal tumor.

Furthermore, for tumors that are resistant to a treatment with an immune modulatory therapy, such as treatment with an immune checkpoint inhibitor, treatment with an anti-PD-L1 conjugate followed by light illumination and/or in combination with an immune checkpoint inhibitor can result in enhanced inhibitory effects on the growth of both illuminated primary tumor and a distal tumor, a primary tumor and a newly arising tumor and/or a primary tumor and a secondary tumor of a different type, indicating a sensitization effect of the anti-PD-L1 photoimmunotherapy on immune checkpoint inhibitors in treating cancers and tumor cells.

### II. CONJUGATES AND COMPOSITIONS FOR USE WITH THE METHODS

In some aspects, provided are compositions, combinations, methods or uses that employ an anti-PD-L1 conjugate that includes a targeting molecule that binds to PD-L1 linked to a phthalocyanine dye. In some aspects, the targeting molecule that binds to PD-L1 is an antibody or antigen-binding fragment thereof. In some embodiments, the targeting molecule binds to PD-L1, such as PD-L1 expressed on the cell surface, for example, of immunosuppressive cells, e.g., M2 TAM, tDCs or MDSCs, and/or certain tumor cells. Also provided are compositions, such as pharmaceutical compositions, that contain any of the conjugates, such as anti-PD-L1 conjugates, described herein, and combinations that contain such compositions or such anti-PD-L1 conjugates. In some aspects, such conjugates, compositions and combinations are for use in a therapy or treatment in accordance with the embodiments provided herein.

In some aspects, an “anti-PD-L1 conjugate” includes a conjugate that has a PD-L1 binding molecule linked to a phthalocyanine dye. The PD-L1 binding molecule can include an anti-PD-L1 antibody or antibody fragment (e.g., antigen-binding fragment), or other protein, peptide or small molecule that binds to PD-L1. In some aspects, exemplary anti-PD-L1 conjugate comprises an antibody or an antigen-binding fragment thereof. Exemplary anti-PD-L1 conjugates include a Si-phthalocyanine dye, such as an IR700 dye.

In some embodiments, the PD-L1 targeting molecule is an antibody or antigen-binding fragment thereof that targets or binds PD-L1, such as an anti-PD-L1 antibody or an antigen-binding fragment thereof. In some aspects, exemplary antibodies that targets or binds to PD-L1 include, but are not limited to, atezolizumab (MPDL3280A, Tecentriq, RG7446)), avelumab (Bavencio), BCD-135, BGB-A333, BMS-936559 (MDX-1105), CBT-502 (TQB-2450), cosibelimab (CK-301), CS1001 (WPB3155), durvalumab (MEDI4736, Imfinzi), FAZ053, HLX20, INBRX-105, KN035, KN046, LDP, LY3300054, LY3415244, M7824 (MSB0011359C), MCLA-145, MSB2311, NM-01, REGN3504, SHR-1316 (HTI-1088), STI-3031 (IMC-001, STI-A1015), TG-1501, ZKAB001 (STI-A1014), and any antigen-binding fragments thereof. Exemplary anti-PD-L1 antibodies include MDX-1105 (MEDAREX), MEDI4736 (Medimmune) MPDL3280A (Genentech), BMS-935559 (Bristol-Myers Squibb) and MSB0010718C and an antigen-binding fragment of any of the foregoing.

In some embodiments, the targeting molecule can be an antibody or antibody fragment that includes the “complementarity-determining regions” or “CDRs” of an anti-PD-L1 antibody, such as any of the described antibodies or antigen-binding fragment thereof. The CDRs are typically responsible for binding to an epitope of an antigen. The CDRs of each chain are typically referred to as CDR1, CDR2, and CDR3, numbered sequentially starting from the N-terminus, and are also generally identified by the chain in which the particular CDR is located. Thus, a heavy chain variable region (V_{H}) CDR3 is located in the variable domain of the heavy chain of the antibody in which it is found, whereas a light chain variable region (V_{L}) CDR1 is the CDR1 from the variable domain of the light chain of the antibody in which it is found. Antibodies with different specificities, such as different combining sites for different antigens, have different CDRs. Although it is the CDRs that vary from antibody to antibody, only a limited number of amino acid positions within the CDRs are directly involved in antigen binding. These positions within the CDRs are called specificity determining residues (SDRs).

The precise amino acid sequence boundaries of a given CDR or framework regions (FR, the non-CDR portions of the variable regions of the heavy and light chains) can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” “J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003, 27(1):55-77 (“IMGT” numbering scheme); Honegger A and Pliickthun A, “Yet another numbering scheme for immunoglobulin variable domains: an automatic modeling and analysis tool,” J Mol Biol, 2001, 309(3):657-70, (“Aho” numbering scheme); and Martin et al., “Modeling antibody hypervariable loops: a combined algorithm,” PNAS, 1989, 86(23):9268-9272, (“AbM” numbering scheme).

In some embodiments, the targeting molecule can be an antibody fragment. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); heavy chain variable region only (VHH) single domain antibodies; and multispecific antibodies formed from antibody fragments. Other antibody fragments or multispecific antibodies formed from antibody fragments include a multivalent scFv, a bispecific scFv or an scFv-CH3 dimer. Antibody fragments can be made by various techniques, including but not limited to proteolytic digestion of an intact antibody as well as production by recombinant host cells.

In some embodiments, the anti-PD-L1 conjugate comprises an antibody or an antigen-binding fragment thereof that comprises the complementary determining regions (CDRs) from an antibody selected from the group consisting of atezolizumab (MPDL3280A, Tecentriq, RG7446)), avelumab (Bavencio), BCD-135, BGB-A333, BMS-936559 (MDX-1105), CBT-502 (TQB-2450), cosibelimab (CK-301), CS1001 (WPB3155), durvalumab (MEDI4736, Imfinzi), FAZ053, HLX20, INBRX-105, KN035, KN046, LDP, LY3300054, LY3415244, M7824 (MSB0011359C), MCLA-145, MSB2311, NM-01, REGN3504, SHR-1316 (HTI-1088), STI-3031 (IMC-001, STI-A1015), TG-1501, and ZKABOO1 (STI-A1014). In some embodiments, the anti-PD-L1 conjugate comprises an antibody or an antigen-binding fragment thereof that comprises the CDRs from an antibody selected from among atezolizumab, avelumab, durvalumab, KN035 or CK-301.

In some of any embodiments, the anti-PD-L1 conjugate comprises an antibody selected from among atezolizumab (MPDL3280A, Tecentriq, RG7446)), avelumab (Bavencio), BCD-135, BGB-A333, BMS-936559 (MDX-1105), CBT-502 (TQB-2450), cosibelimab (CK-301), CS1001 (WPB3155), durvalumab (MEDI4736, Imfinzi), FAZ053, HLX20, INBRX-105, KN035, KN046, LDP, LY3300054, LY3415244, M7824 (MSB0011359C), MCLA-145, MSB2311, NM-01, REGN3504, SHR-1316 (HTI-1088), STI-3031 (IMC-001, STI-A1015), TG-1501, ZKABOO1 (STI-A1014), and any antigen-binding fragments thereof. Exemplary anti-PD-L1 antibodies include MDX-1105 (MEDAREX), MEDI4736 (Medimmune) MPDL3280A (Genentech), BMS-935559 (Bristol-Myers Squibb) and MSB0010718C or an antigen-binding fragment thereof. In some embodiments, the anti-PD-L1 conjugate comprises an antibody selected from among atezolizumab, avelumab, durvalumab, KN035 and CK-301, or an antigen-binding fragment thereof.

In some embodiments, the antibody of the conjugate is a biosimilar, interchangeable or biobetter of any of the anti-PD-L1 antibody described herein, e.g., atezolizumab, avelumab, durvalumab, KN035 or CK-301 or an antigen-binding fragment thereof. Such antibodies also include copy biologicals and biogenerics of any of the anti-PD-L1 antibody described herein, e.g., atezolizumab, avelumab, durvalumab, KN035 or CK-301 or an antigen-binding fragment thereof.

In some embodiments, the targeting molecule of an anti-PD-L1 antibody comprises a functional Fc region. In some embodiments, the targeting molecule that is an anti-PD-L1 antibody does not comprise a functional Fc region. In some embodiments, the targeting molecule that is an anti-PD-L1 antibody is a humanized antibody. In some embodiments, the targeting molecule that is an anti-PD-L1 antibody is a fully human antibody.

In some aspects, the anti-PD-L1 conjugates employed in the provided embodiments include a phthalocyanine dye. In some embodiments, the phthalocyanine dye is a phthalocyanine dye with a silicon coordinating metal (Si-phthalocyanine dye). In some embodiments, the phthalocyanine dye comprises the formula:

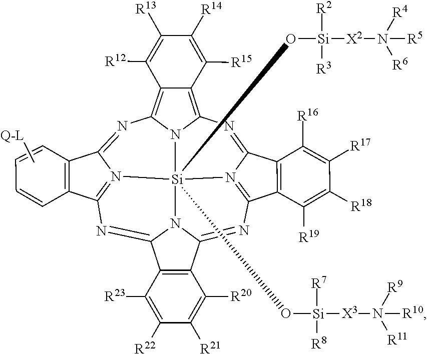

wherein:
L is a linker;
Q is a reactive group for attachment of the dye to the targeting molecule;
R², R³, R⁷, and R⁸ are each independently selected from among optionally substituted alkyl and optionally substituted aryl;
R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group;
R¹², R¹³, R¹⁴, R¹⁵, R¹⁶, R¹⁷, R¹⁸, R¹⁹, R²⁰, R²¹, R²² and R²³ are each independently selected from among hydrogen, halogen, optionally substituted alkylthio, optionally substituted alkylamino and optionally substituted alkoxy; and
X² and X³ are each independently C₁-C₁₀ alkylene, optionally interrupted by a heteroatom.

In some embodiments, the phthalocyanine dye comprises the formula:

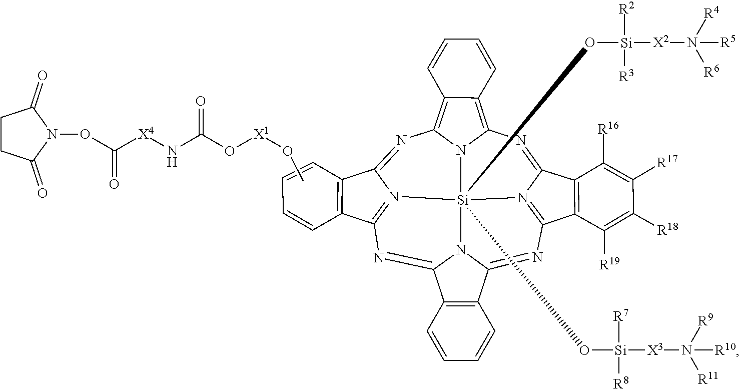

wherein:
X¹ and X⁴ are each independently a C₁-C₁₀ alkylene optionally interrupted by a heteroatom;
R², R³, R⁷, and R⁸ are each independently selected from optionally substituted alkyl and optionally substituted aryl;
R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group; and
R¹⁶, R¹⁷, R¹⁸ and R¹⁹ are each independently selected from among hydrogen, halogen, optionally substituted alkylthio, optionally substituted alkylamino and optionally substituted alkoxy.

In some embodiments of the methods and uses provided herein, a Si-phthalocyanine dye is IRDye 700DX (IR700). In some embodiments, the phthalocyanine dye containing the reactive group is IR700 NHS ester, such as IRDye 700DX NHS ester (LiCor 929-70010, 929-70011). In some embodiments, the dye is a compound having the following formula:

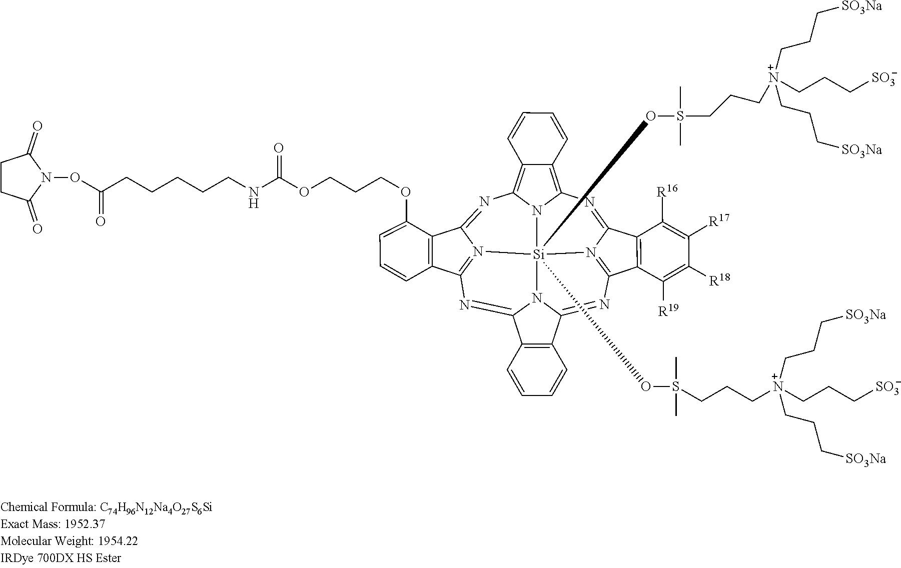

For purposes herein, the term “IR700,” “IRDye 700” or “IRDye 700DX” includes the above formula when the dye is conjugated such as to an antibody, e.g. via a reactive group.

In some embodiments, the conjugate for use with the methods herein include an anti-PD-L1 conjugate comprising a Si-phthalocyanine dye linked to targeting molecule that binds to PD-L1. In some embodiments, the conjugate is an anti-PD-L1 antibody-Si-phthalocyanine dye conjugate. In some embodiments, the conjugate is an anti-PD-L1 antibody-IR700 conjugate. In some embodiments, the conjugate is an anti-PD-L1 antibody-IR700 conjugate, where the antibody is atezolizumab, avelumab, durvalumab, KN035 or CK-301, or an antigen-binding fragment thereof. In some embodiments, the conjugate is an atezolizumab-IR700 conjugate. In some embodiments, the conjugate is an avelumab-IR700 conjugate. In some embodiments, the conjugate is a durvalumab-IR700 conjugate. In some embodiments, the conjugate is a KN035-IR700 conjugate. In some embodiments, the conjugate is a CK-301-IR700 conjugate.

### III. METHODS OF ADMINISTRATION AND FORMULATIONS

In some embodiments, the anti-PD-L1 conjugate may be administered either systemically or locally to the organ or tissue to be treated. Exemplary routes of administration include, but are not limited to, topical, injection (such as subcutaneous, intramuscular, intradermal, intraperitoneal, intratumoral, and intravenous), oral, sublingual, rectal, transdermal, intranasal, vaginal and inhalation routes. In some embodiments, the anti-PD-L1 conjugate is administered intravenously. In some embodiments, the anti-PD-L1 conjugate is administered parenterally. In some embodiments, the anti-PD-L1 conjugate is administered enterally. In some embodiments, the conjugate is administered by local injection. In some embodiments, the conjugate is administered as a topical application.

The compositions comprising the anti-PD-L1 conjugate can be administered locally or systemically using any method known in the art, for example to subjects having a tumor, such as a cancer, or who has had a tumor previously removed, for example via surgery. Although specific examples are provided, one skilled in the art will appreciate that alternative methods of administration of the disclosed agents can be used. Such methods may include for example, the use of catheters or implantable pumps to provide continuous infusion over a period of several hours to several days into the subject in need of treatment.

In some embodiments, the anti-PD-L1 conjugate is administered by parenteral means, including direct injection or infusion into a tumor, such as intratumorally. In some embodiments, the anti-PD-L1 conjugate is administered to the tumor by applying the agent to the tumor, for example by bathing the tumor in a solution containing the anti-PD-L1 conjugate, or by pouring the agent onto the tumor.

In addition, or alternatively, the anti-PD-L1 conjugate can be administered systemically, for example intravenously, intramuscularly, subcutaneously, intradermally, intraperitoneally, subcutaneously, or orally, to a subject having a tumor, such as cancer.

Also provided herein are compositions, such as pharmaceutical compositions, containing the anti-PD-L1 conjugate, and uses of such compositions, such as therapeutic uses and/or uses as a medicament. In some aspects, the compositions comprise the anti-PD-L1 conjugate and a pharmaceutically acceptable carrier. In some embodiments, the composition containing the anti-PD-L1 conjugate is for use in treatment or therapy, in accordance with any of the provided embodiments, such as for administration to a subject having a disease or condition, for the treatment of the disease or condition. The dosages of the anti-PD-L1 conjugate to be administered to a subject are not subject to absolute limits, but will depend on the nature of the composition and its active ingredients and its unwanted side effects, such as immune response against the agent, the subject being treated, and the type of condition being treated and the manner of administration. Generally, the dose will be a therapeutically effective amount, such as an amount sufficient to achieve a desired biological effect, for example an amount that is effective to decrease the size, such as volume and/or weight, of the tumor, or attenuate further growth of the tumor, or decrease undesired symptoms of the tumor.

In some embodiments, the compositions used for administration of the anti-PD-L1 conjugate contain an effective amount of the agent along with conventional pharmaceutical carriers and excipients appropriate for the type of administration contemplated. For example, in some embodiments, parenteral formulations may contain a sterile aqueous solution or suspension of the conjugate. In some embodiments, compositions for enteral administration may contain an effective amount of the anti-PD-L1 conjugate in aqueous solution or suspension that may optionally include buffers, surfactants, thixotropic agents, and flavoring agents.

In some embodiments, the anti-PD-L1 conjugate or conjugate in combination with an additional therapeutic agent, can be formulated in a pharmaceutically acceptable buffer, such as that containing a pharmaceutically acceptable carrier or vehicle. Generally, the pharmaceutically acceptable carriers or vehicles, such as those present in the pharmaceutically acceptable buffer, are can be any known in the art. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds. Pharmaceutically acceptable compositions generally are prepared in view of approvals for a regulatory agency or other agency prepared in accordance with generally recognized pharmacopeia for use in animals and in humans.

Pharmaceutical compositions can include carriers such as a diluent, adjuvant, excipient, or vehicle with which the compound is administered. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyvinylpyrrolidone, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents.

In some embodiments, pharmaceutical preparation can be in liquid form, for example, solutions, syrups or suspensions. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In some cases, pharmaceutical preparations can be presented in lyophilized form for reconstitution with water or other suitable vehicle before use.

In some embodiments, the nature of the pharmaceutically acceptable buffer, or carrier, depends on the particular mode of administration being employed. For instance, in some embodiments, parenteral formulations may comprise injectable fluids that include pharmaceutically and physiologically acceptable fluids such as water, physiological saline, balanced salt solutions, aqueous dextrose, or glycerol as a vehicle. In some embodiments, for solid compositions, for example powder, pill, tablet, or capsule forms, non-toxic solid carriers can include, for example, pharmaceutical grades of mannitol, lactose, starch, or magnesium stearate. In addition to biologically neutral carriers, pharmaceutical compositions to be administered can in some embodiments contain minor amounts of non-toxic auxiliary substances, such as wetting or emulsifying agents, preservatives, and pH buffering agents, for example sodium acetate or sorbitan monolaurate.

The compounds can be formulated into suitable pharmaceutical preparations such as solutions, suspensions, tablets, dispersible tablets, pills, capsules, powders, sustained release formulations or elixirs, for oral administrate, as well as transdermal patch preparation and dry powder inhalers. Typically, the compounds are formulated into pharmaceutical compositions using techniques and procedures well known in the art (see e.g., Ansel Introduction to Pharmaceutical Dosage Forms, Fourth Edition, 1985, 126). Generally, the mode of formulation is a function of the route of administration.

Compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. Other modes of administration also are contemplated. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant.

Parenteral administration, generally characterized by injection, either subcutaneously, intramuscularly, intratumorally, intravenously or intradermally is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain an activator in the form of a solvent such as pH buffering agents, metal ion salts, or other such buffers. The pharmaceutical compositions also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) also is contemplated herein. The percentage of active compound contained in such parenteral compositions is highly dependent on the specific nature thereof, as well as the activity of the compound and the needs of the subject.

Injectables are designed for local and systemic administration. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or non-aqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, non-aqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Non-aqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate.

If administered intravenously, suitable carriers include physiological saline or phosphate buffered saline (PBS), and solutions containing thickening and solubilizing agents, such as glucose, polyethylene glycol, and polypropylene glycol and mixtures thereof.

The composition can be formulated for single dosage administration or for multiple dosage administration. The agents can be formulated for direct administration. The composition can be provided as a liquid or lyophilized formulation. Where the composition is provided in lyophilized form it can be reconstituted just prior to use by an appropriate buffer, for example, a sterile saline solution.

Compositions also can be administered with other biologically active agents, either sequentially, intermittently or in the same composition. Administration also can include controlled release systems including controlled release formulations and device-controlled release, such as by means of a pump.

The most suitable route in any given case depends on a variety of factors, such as the nature of the disease, the progress of the disease, the severity of the disease and the particular composition which is used. For example, compositions are administered systemically, for example, via intravenous administration. Subcutaneous methods also can be employed, although increased absorption times can be necessary to ensure equivalent bioavailability compared to intravenous methods.

Pharmaceutical compositions can be formulated in dosage forms appropriate for each route of administration. Pharmaceutically and therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Unit dosage forms include, but are not limited to, tablets, capsules, pills, powders, granules, sterile parenteral solutions or suspensions, and oral solutions or suspensions, and oil water emulsions containing suitable quantities of the compounds or pharmaceutically acceptable derivatives thereof. Unit dose forms can be contained ampoules and syringes or individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared. Pharmaceutical compositions can be formulated in dosage forms appropriate for each route of administration.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. All preparations for parenteral administration must be sterile, as is known and practiced in the art. In some embodiments, the compositions can be provided as a lyophilized powder, which can be reconstituted for administration as solutions, emulsions and other mixtures. They may also be reconstituted and formulated as solids or gels. The lyophilized powders can be prepared from any of the solutions described above.

The sterile, lyophilized powder can be prepared by dissolving a phthalocyanine dye-targeting molecule conjugate in a buffer solution. The buffer solution may contain an excipient which improves the stability of other pharmacological components of the powder or reconstituted solution, prepared from the powder.

In some embodiments, subsequent sterile filtration of the solution followed by lyophilization under standard conditions known to those of skill in the art provides the desired formulation. Briefly, the lyophilized powder is prepared by dissolving an excipient, such as dextrose, sorbitol, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to ensure sterility and apportioned into vials for lyophilization. Each vial can contain a single dosage (1 mg-1 g, generally 1-100 mg, such as 1-5 mg) or multiple dosages of the compound. The lyophilized powder can be stored under appropriate conditions, such as at about 4 ° C. to room temperature. Reconstitution of this lyophilized powder with a buffer solution provides a formulation for use in parenteral administration. The precise amount depends upon the indication treated and selected compound. Such amount can be empirically determined.

In some embodiments, the pH of the composition is between or between about 6 and 10, such as between or between about 6 and 8, between or between about 6.9 and 7.3, such as about pH 7.1. In some embodiments, the pH of the pharmaceutically acceptable buffer is at least or about 5, at least or about 6, at least or about 7, at least or about 8, at least or about 9 or at least or about 10, or is 7.1.

The compositions can be formulated for single dosage administration or for multiple dosage administration. The agents can be formulated for direct administration.

In some embodiments, the compositions provided herein are formulated in an amount for direct administration of the anti-PD-L1 conjugate, in a range from at or about 0.01 mg to at or about 3000 mg, from at or about 0.01 mg to at or about 1000 mg, from at or about 0.01 mg to at or about 500 mg, from at or about 0.01 mg to at or about 100 mg, from at or about 0.01 mg to at or about 50 mg, from at or about 0.01 mg to at or about 10 mg, from at or about 0.01 mg to at or about 1 mg, from at or about 0.01 mg to at or about 0.1 mg, from at or about 0.1 mg to at or about 2000 mg, from at or about 0.1 mg to at or about 1000 mg, from at or about 0.1 mg to at or about 500 mg, from at or about 0.1 mg to at or about 100 mg, from at or about 0.1 mg to at or about 50 mg, from at or about 0.1 mg to at or about 10 mg, from at or about 0.1 mg to at or about 1 mg, from at or about 1 mg to at or about 2000 mg, from at or about 1 mg to at or about 1000 mg, from at or about 1 mg to at or about 500 mg, from at or about 1 mg to at or about 100 mg, from at or about 1 mg to at or about 10 mg, from at or about 10 mg to at or about 2000 mg, from at or about 10 mg to at or about 1000 mg, from at or about 10 mg to at or about 500 mg, from at or about 10 mg to at or about 100 mg, from at or about 100 mg to at or about 2000 mg, from at or about 100 mg to at or about 1000 mg, from at or about 100 mg to at or about 500 mg, from at or about 500 mg to at or about 2000 mg, from at or about 500 mg to at or about 1000 mg, and from about 1000 mg to at or about 3000 mg. In some embodiments, the volume of the composition can be 0.5 mL to 1000 mL, such as 0.5 mL to 100 mL, 0.5 mL to 10 mL, 1 mL to 500 mL, 1 mL to 10 mL, such as at least or about at least or about or 0.5 mL, 1 mL, 2 mL, 3 mL, 4 mL, 5 mL, 6 mL, 7 mL, 8 mL, 9 mL, 10 mL, 15 mL, 20 mL, 30 mL, 40 mL, 50 mL or more. For example, the composition is formulated for single dosage administration of an amount between at or about 100 mg and at or about 500 mg, or between at or about 200 mg and at or about 400 mg. In some embodiments, the composition is formulated for single dosage administration of an amount between at or about 500 mg and at or about 1500 mg, at or about 800 mg and at or about 1200 mg or at or about 1000 mg and at or about 1500 mg. In some embodiments, the volume of the composition is between at or about 10 mL and at or about 1000 mL or at or about 50 mL and at or about 500 mL; or the volume of the composition is at least at or about 10 mL, 20 mL, 30 mL, 40 mL, 50 mL, 75 mL, 100 mL, 150 mL, 200 mL, 250 mL, 300 mL, 400 mL, 500 mL or 1000 mL.

In some embodiments, the entire vial contents of the formulations can be withdrawn for administration or can be divided up into a plurality of dosages for multiple administrations. Upon withdrawal of an amount of drug for administration, the formulation can be further diluted if desired, such as diluted in water, saline (e.g., 0.9%) or other physiological solution.

In some embodiments, also provided are compositions containing an additional therapeutic agent, such as an immunomodulatory agent or anti-cancer agent, for use in combination with the anti-PD-L1 conjugate, in accordance with the provided embodiments. In some aspects, the additional therapeutic agent can be prepared in accord with known or standard formulation guidelines, such as described above. In some embodiments, the immunomodulatory agent, anti-cancer agent and/or anti-PD-L1 conjugate are formulated as separate compositions. In some embodiments, the immunomodulatory agent is provided as a separate composition from the anti-PD-L1 conjugate, and the two compositions are administered separately. In some embodiments, the anti-cancer agent is provided as a separate composition from the anti-PD-L1 conjugate, and the two compositions are administered separately. The compositions can be formulated for parenteral delivery (i.e. for systemic delivery). For example, the compositions or combination of compositions are formulated for subcutaneous delivery or for intravenous delivery. The agents, such as an anti-PD-L1 conjugate, and an immunomodulatory agent and/or an anti-cancer agent can be administered by different routes of administration.

In some aspects, exemplary additional therapeutic agents, such as immunomodulatory agents, can be administered as directed for a monotherapy or on other administration schedules and dose for the particular therapeutic agent. In some embodiments of the methods and uses that involve administration of with an anti-PD-L1 conjugate and an additional therapeutic agent, the additional therapeutic agent is administered at the recommended dose and/or schedule of administration. In some embodiments, an additional therapeutic agent can be administered in the methods herein at a dose lower than the recommended amount or on an alternate schedule, such as when anti-PD-L1 conjugate sensitizes a tumor or cancer or the TME to the additional therapeutic agent and/or when the combination of an anti-PD-L1 conjugate and an additional therapeutic agent results in a synergistic response.

### IV. DEVICES AND ILLUMINATION METHODS FOR USE WITH THE ANTI-PD-L1 CONJUGATES

In some aspects, devices that can be used with the provided embodiments include light diffusing devices that provide illumination (in some cases, also referred to as irradiation) at a wavelength (or wavelengths) of light wavelength suitable for use with the dye conjugate composition, such as a phthalocyanine dye conjugate (e.g., an anti-PD-L1 conjugate such as those described herein). Illumination devices can include a light source (for example, a laser), and a means of conveying the light to the area of interest (for example, one or more fibers to illuminate an isolated area of a subject or an isolated lesion or tumor).

In some embodiments, the target area, such as a tumor, the vicinity of a tumor, a lymph node, the vicinity of the lymph node, is illuminated with light at a wavelength within a range from at or about 400 nm to at or about 900 nm, such as from or from at or about 500 nm to at or about 900 nm, such as from or from at or about 600 nm to at or about 850 nm, such as from or from at or about 600 nm to at or about 740 nm, such as from at or about 660 nm to at or about 740 nm, from at or about 660 nm to at or about 710 nm, from at or about 660 nm to at or about 700 nm, from at or about 670 nm to at or about 690 nm, from at or about 680 nm to at or about 740 nm, or from at or about 690 nm to at or about 710 nm. In some embodiments, the target area, such as a tumor, the vicinity of a tumor, a lymph node, the vicinity of the lymph node, or the tumor microenvironment, is illuminated with light at a wavelength of at or about 600 nm to at or about 850 nm, such as at or about 660 nm to at or about 740 nm. In some embodiments, the target area, such as a tumor, the vicinity of a tumor, a lymph node, the vicinity of the lymph node, or the tumor microenvironment, is illuminated with light at wavelength of at least at or about 600 nm, 620 nm, 640 nm, 660 nm, 680, nm, 700 nm, 720 nm or 740 nm, such as at or about 690 ±50 nm, or at or about 690 ±40 nm, for example at or about 690 nm or at or about 680 nm.

In some embodiments of the methods and uses provided herein, illumination is carried out using cylindrical diffusing fibers that includes a diffuser length of at or about 0.5 cm to at or about 10 cm and spaced at or about 1.8 ±0.2 cm apart. In some embodiments, the light illumination dose is from at or about 20 J/cm fiber length to at or about 500 J/cm fiber length. In some embodiments, the tumor is greater than at or about 10 mm deep or is a subcutaneous tumor.

In some embodiments, the provided methods include illuminating a target area that is an interstitial tumor in a subject with cylindrical diffusing fibers that includes a diffuser length of at or about 0.5 cm to at or about 10 cm and spaced at or about 1.8±0.2 cm apart with a light dose of at or about 100 J/cm fiber length or with a fluence rate of at or about 400 mW/cm. In some embodiments, the target area is a tumor that is greater than at or about 10 mm deep or is a subcutaneous tumor. In some embodiments, the cylindrical diffusing fibers are placed in a catheter positioned in the tumor at or about 1.8±0.2 cm apart. In some embodiments, the catheter is optically transparent.

In some embodiments, the target area, such as a tumor, the vicinity of a tumor, a lymph node, the vicinity of the lymph node, or the tumor microenvironment, is illuminated with light dose of at least at or about 1 J/cm², such as at least at or about 10 J/cm², at least at or about 30 J/cm², at least at or about 50 J/cm², at least at or about 100 J/cm², or at least at or about 500 J/cm². In some embodiments, the dose of illumination is from at or about 1 to at or about J/cm², from at or about 1 to at or about 500 J/cm², from at or about 5 to at or about 200 J/cm², from at or about 10 to at or about 100 J/cm², or from at or about 10 to at or about 50 J/cm². In some embodiments, the target area is illuminated at a dose of at least at or about 2 J/cm², 5 J/cm², 10 J/cm², 25 J/cm², 50 J/cm², 75 J/cm², 100 J/cm², 150 J/cm², 200 J/cm², 300 J/cm², 400 J/cm², or 500 J/cm².

In some embodiments, the target area is a tumor that is a superficial tumor. In some embodiments, the tumor is less than 10 mm thick. In some embodiments, illumination is carried out using a microlens-tipped fiber for surface illumination. In some embodiments, the light illumination dose is from at or about 5 J/cm² to at or about 200 J/cm².

In some embodiments, the target area, such as a tumor, the vicinity of a tumor, a lymph node, the vicinity of the lymph node, or the tumor microenvironment, are illuminated at a dose of at least at or about 1 J/cm fiber length, such as at least at or about 10 J/cm fiber length, at least at or about 50 J/cm fiber length, at least at or about 100 J/cm fiber length, at least at or about 250 J/cm fiber length, or at least at or about 500 J/cm fiber length. In some embodiments, the dose of illumination is from at or about 1 to at or about 1000 J/cm fiber length, from at or about 1 to at or about 500 J/cm fiber length, from at or about 2 to at or about 500 J/cm fiber length, from at or about 50 to at or about 300 J/cm fiber length, from at or about 10 to at or about 100 J/cm fiber length, or from at or about 10 to at or about 50 J/cm fiber length. In some embodiments, the target area, such as a tumor, the vicinity of a tumor, a lymph node, the vicinity of the lymph node, or the tumor microenvironment, are illuminated at a dose of at least at or about 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length.

In some embodiments, the provided methods include illuminating a target area that is a superficial tumor in a subject with a microlens-tipped fiber for surface illumination with a light dose of from at or about 5 J/cm² to at or about 200 J/cm². In some embodiments, the light illumination dose is at or about 50 J/cm².

In some cases, it is found that a dose of illumination in a human subject to achieve PIT can be less than is necessary for PIT in a mouse. For example, in some cases, at or about 50 J/cm² (50 J/cm²) light dosimetry in an in vivo tumor mouse model is not effective for PIT, which is in contrast to what we can be observed in the clinic with human patients.

In some embodiments, the dose of illumination following administration of the composition comprising the phthalocyanine dye-targeting molecule conjugate is at least at or about 1 J/cm² or at least at or about 1 J/cm of fiber length at a wavelength of at or about 660-740 nm, for example, at least at or about 10 J/cm² or at least at or about 10 J/cm of fiber length at a wavelength of at or about 660-740 nm, at least at or about 50 J/cm² or at least at or about 50 J/cm of fiber length at a wavelength of at or about 660-740 nm, or at least at or about 100 J/cm²or at least at or about 100 J/cm of fiber length at a wavelength of at or about 660-740 nm. In some embodiments, the wavelength is 660-710 nm. In some embodiments, the dose of illumination following administration of the composition comprising the phthalocyanine dye-targeting molecule conjugate is at least at or about 1.0 J/cm² or at least at or about 1 J/cm of fiber length, at a wavelength of at or about 690 nm, for example, at least at or about 10 J/cm² or at least at or about 10 J/cm of fiber length, at a wavelength of at or about 690 nm, at least at or about 50 J/cm² or at least at or about 50 J/cm of fiber length, at a wavelength of at or about 690 nm, or at least at or about 100 J/cm² or at least at or about 100 J/cm of fiber length, at a wavelength of at or about 690 nm, for example 1.0 to 500 J/cm² or 1.0 to 500 J/cm of fiber length, at a wavelength of at or about 690 nm. Exemplary illumination after administration of the conjugates or compositions provided herein include illuminating the target area at a wavelength of at or about 660 nm to at or about 740 nm at a dose of at least at or about 1 J/cm² or at least at or about 1 J/cm of fiber length.

In some embodiments, illuminating is carried out at a wavelength of at or about 600 nm to at or about 850 nm and at a dose of from at or about 25 J/cm² to at or about 400 J/cm² or from at or about 2 J/cm fiber length to at or about 500 J/cm fiber length. In some embodiments, the target area is illuminated at a wavelength of 690 ±40 nm. In some embodiments, target area is illuminated at a dose of at or about of 50 J/cm² or at or about 100 J/cm of fiber length.

In some embodiments, a light or laser may be applied to the dye molecules, such as cells containing the conjugate, for from at or about 5 seconds to at or about 5 minutes. For example, in some embodiments, the light or laser is applied for at or about 5, 10, 15, 20, 25, 30, 35, 40, 45 50 or 55 seconds, or for within a range between any of two such values, to activate the dye molecules. In some embodiments, the light or laser is applied for at or about 1, 1.5, 2, 2.5, 3, 3.5, 4, 4.5 or 5 minutes, or more, or within a range between any two of such values. In some embodiments, the length of time a light or laser is applied can vary depending, for example, on the energy, such as wattage, of the light or laser. For example, lights or lasers with a lower wattage may be applied for a longer period of time in order to activate the dye molecule.

In some embodiments, a light or laser may be applied for at or about 30 minutes to at or about 96 hours after administering the conjugate. For example, in some embodiments, the light or laser is applied at or at about 30, 35, 40, 45, 50 or 55 minutes after administering the conjugate, or within a range between any two of such values. In some embodiments, the light or laser is applied at or at about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours after administering the conjugate, or is administered within a range between about any two of such values, such as, for example between at or about 20 hours to at or about 28 hours, or about 24 hours ±4 hours. In some embodiments, the light or laser is applied between or between about 1 and 24 hours, such as between at or about 1 and at or about 12 hours, at or about 12 and at or about 24 hours, at or about 6 and at or about 12 hours, or may be administered more than at or about 24 hours following administration of the conjugate. In some embodiments, the light or laser is applied at or about 36, 48, 72 or 96 hours after administering the conjugate. In some embodiments, the light or laser is applied at or at about 24 hours ±4 hours after administering the conjugate.

In some embodiments, the target area, such as a tumor, the vicinity of a tumor, a lymph node, the vicinity of the lymph node, or the tumor microenvironment, or subjects, can be illuminated one or more times. Thus, illumination can be completed in a single day, or can be done repeatedly on multiple days with the same or a different dosage, such as illumination at least at or about 2 different times, 3 different times, 4 different times 5 different times or 10 different times. In some embodiments, repeated illuminations may be done on the same day, on successive days, or every 1-3 days, every 3-7 days, every 1-2 weeks, every 2-4 weeks, every 1-2 months, or at even longer intervals. In some embodiments, multiple illuminations are performed, such as at least 2, at least 3, or at least 4 illuminations, such as 2, 3, 4, 5, 6, 7, 8, 9 or 10 separate administrations.

In some embodiments, the dose or method of illumination differs depending on the type or morphology of the target area, such as a tumor, the vicinity of a tumor, a lymph node, the vicinity of the lymph node.

In some embodiments, the illumination employs a device with “top hat” irradiance distribution profile, such as those described in W02018/080952 and US20180239074.

### V. COMBINATION THERAPY

In some embodiments, also provided are methods and uses that include combination therapies, and combinations, such as combinations for use in accordance with the combination therapy. In some aspects, the combinations include an anti-PD-L1 conjugate and an additional therapeutic agent, such as an immunomodulatory agent or an anti-cancer agent. In some embodiments, the targeting molecule used for an anti-PD-L1 conjugate in such combination therapies is an anti-PD-L1 antibody, or an antibody fragment that binds to PD-L1. In some embodiments, the conjugate is an anti-PD-L1 antibody, or an antibody fragment that binds to PD-L1 linked to a Si-phthalocyanine dye, such as an IR700 dye. In some aspects, the combination therapy includes administration of the anti-PD-L1 conjugate and the additional therapeutic, e.g., an immunomodulatory agent or an anti-cancer agent. In such methods, the primary tumors, newly arising tumors, invasive tumor cells, and metastatic tumor cells can be sensitized to the treatment with the additional therapeutic agent, such as an immunomodulatory agent or an anti-cancer agent. In such methods, the growth of primary tumors, newly arising tumors, invasive tumor cells, and metastatic tumor cells can be inhibited, reduced or eliminated, and/or the volume of one or more tumors is reduced.

The increase in sensitivity as a result of such combination treatments can include, but not limited to, a reduction of inhibition of tumor growth, a reduction in tumor cell invasion and/or metastasis, an increase in tumor cell killing, an increase in systemic immune response, an increase in new T cell priming, an increase in the diversity of intratumoral CD8⁺ T cells, an increase in the number and/or activity of intratumoral CD8⁺ T effector cells, a decrease in the number and/or activity of intratumoral regulatory T cells, a decrease in the number and/or activity of intratumoral myeloid derived suppressor cells, a decrease in the number and/or activity of intratumoral tumor associated fibroblasts or cancer associated fibroblasts (CAFs), or any combination thereof.

In some embodiments the additional therapeutic agent is an anticancer agent. In some embodiments, the anticancer agent can be one or more of a chemotherapeutic agent, an antibody treatment, and a radiotherapeutic agent. In some embodiments, the additional therapeutic agent is an anti-cancer agent selected from a checkpoint inhibitor, an immune adjuvant, a chemotherapeutic agent, radiation, and a biologic comprising an anti-cancer targeting molecule that binds to a tumor cell.

In some aspects, the additional therapeutic agent is an immunomodulatory agent (also called immune modulating agent), such as an immune checkpoint inhibitor. In some aspects, such combination is employed for treatment of the tumor, lesion or cancer. In some embodiments, the methods include the administration of the immunomodulatory agent, such as an immune checkpoint inhibitor, prior to, concurrent with or subsequent to the administration of an anti-PD-L1 conjugate.

In some embodiments, the additional therapeutic agent, such as an immunomodulatory agent, used in such combination therapies herein can include an adjuvant, immune checkpoint inhibitor, cytokine or any combination thereof. A cytokine for use in the combinations can be, for example, Aldesleukin (PROLEUKIN), Interferon alfa-2a, Interferon alfa-2b (Intron A), Peginterferon Alfa-2b (SYLATRON/PEG-Intron), or a cytokine that targets the IFNAR1/2 pathway, the IL-2/IL-2R pathway. An adjuvant for use in the combinations can be, for example, Poly ICLC (HILTONOL/Imiquimod), 4-1BB (CD137; TNFRS9), OX40 (CD134) OX40-Ligand (OX40L), Toll-Like Receptor 2 Agonist SUP3, Toll-Like Receptor TLR3 and TLR4 agonists and adjuvants targeting the Toll-like receptor 7 (TLR7) pathway, other members of the TNFR and TNF superfamilies, other TLR2 agonists, TLR3 agonists and TLR4 agonists.

In some embodiments, the additional therapeutic agent is an immune checkpoint inhibitor that is a PD-1 inhibitor, such as a small molecule, antibody or antigen binding fragment. Exemplary anti-PD-1 antibodies include, but are not limited to, pembrolizumab (MK-3475, Keytruda), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), GLS-010, CS1003, LZMO09, camrelizumab(SHR-1210), SCT-I10A, MGA012, AK105, PF-06801591, AMP-224, AB122, AMG 404, BI 754091, HLX10, JTX-4014, MEDI0680, Sym021, MGD019, MGD013, AK104, XmAb20717, R07121661, CX-188, and spartalizumab.

In some embodiments, the additional therapeutic agent is an immune checkpoint inhibitor that is a CTLA-4 inhibitor, such as a small molecule, antibody or antigen binding fragment. In some of any embodiments, the anti-CTLA-4 antibody is selected from the group consisting of ipilimumab (YERVOY), tremelimumab, AGEN1181, AGEN1884, ADU-1064, BCD-145, and BCD-217.

In some embodiments, the additional therapeutic agent is a CD25 inhibitor, such as a small molecule, antibody or antigen binding fragment. In some of any embodiments, the anti-CD25 antibody is selected from the group consisting of basiliximab (Simulect®), daclizumab, PC61.

The administration of an additional therapeutic agent, such as a checkpoint inhibitor, adjuvant or cytokine, can be administered prior to, concurrent with, or subsequent to the administration of the anti-PD-L1 conjugate. For example, the methods can include administering one or more doses of an immune checkpoint inhibitor, administering an anti-PD-L1 conjugate, and after administration of the conjugate, illuminating with a suitable wavelength of light a target area. The methods can include first administering the conjugate, and after administration of the conjugate, illuminating a target area, and then administering an additional therapeutic agent, such as an immune checkpoint inhibitor, subsequently either to administration of the conjugate or subsequently to the illumination step. The methods can also include the administration of an additional therapeutic agent, such as an immune checkpoint inhibitor, concurrently with administration of the conjugate, followed by illuminating a target area. In some embodiments, an additional therapeutic agent, such as an immune checkpoint inhibitor, adjuvant or cytokine, is administered one or more times, prior to when an anti-PD-L1 conjugate is administered, followed by illuminating a target area, and then one or more additional administrations of an additional therapeutic agent (the same or a different an additional therapeutic agent).

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “conjugate” refers to a targeting molecule linked directly or indirectly to a photoactivatable dye, such as those produced by chemical conjugates and those produced by any other methods. For example, a conjugate can refer to a phthalocyanine dye, such as a silicon-phthalocyanine dye (Si-phthalocyanine dye), such as an IR700 molecule, linked directly or indirectly to one or more targeting molecules, such as to a polypeptide binds to or targets to a cell surface protein. A targeting molecule can be a peptide, a polypeptide, more than one polypeptide, an antibody, a portion of an antibody (such as an antigen binding fragment), or a chemical moiety.

As used herein an “anti-PD-L1 conjugate” refers to a conjugate having a targeting molecule that binds to PD-L1. An anti-PD-L1 conjugate can have a targeting molecule that is an antibody, antigen-binding fragment, small molecule, peptide, polypeptide or other moiety that binds to PD-L1.

As used herein, an “antibody” refers to a polypeptide comprising at least a light chain or heavy chain immunoglobulin variable region which specifically recognizes and binds an epitope of an antigen, such as a tumor-specific protein. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. The term “antibody” also includes intact antibodies and antigen-binding antibody fragments that exhibit antigen binding, such as Fab fragments, Fab′ fragments, F(ab)′₂ fragments, Fab′-SH fragments, single chain Fv proteins (“scFv”), heavy chain variable region only (VHH) single domain antibodies, and disulfide stabilized Fv proteins (“dsFv”); diabodies; linear antibodies; and multispecific antibodies formed from antibody fragments. Other antibody fragments or multispecific antibodies formed from antibody fragments include a multivalent scFv, a bispecific scFv or an scFv-CH3 dimer. An scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term “antibody” also includes genetically engineered forms such as modified forms of immunoglobulins, chimeric antibodies, for example, humanized murine antibodies, and heteroconjugate antibodies, such as bispecific antibodies. See also, Pierce Catalog and Handbook, 1994-1995 (Pierce Chemical Co., Rockford, Ill.); Kuby, J., Immunology, 3^{rd} Ed., W.H. Freeman & Co., New York, 1997.

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

“Specifically binds” refers to the ability of individual antibodies to specifically immunologically react with an antigen, such as PD-L1, relative to binding to unrelated proteins, such as non-tumor proteins, for example β-actin. For example, a PD-L1-specific binding agent binds substantially only the PD-L1 protein in vitro or in vivo. As used herein, the term “tumor-specific binding agent” includes tumor-specific antibodies and other agents that bind substantially only to a tumor-specific protein in that preparation.

“Antibody-IR700 molecule” or “antibody-IR700 conjugate” refers to a molecule that includes both an antibody, such as a tumor-specific antibody, conjugated to IR700. In some examples the antibody is a humanized antibody (such as a humanized monoclonal antibody) that specifically binds to a surface protein on a cancer cell.

“Antigen” refers to a compound, composition, or substance that can stimulate the production of antibodies or a T cell response in an animal, including compositions (such as one that includes a tumor-specific protein) that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. In some examples, an antigen includes a tumor-specific peptide (such as one found on the surface of a cancer cell) or immunogenic fragment thereof.

“Immunomodulatory agent” and “immune modulatory therapy” refer to a therapeutic agent and treatment with such agent, respectively that modulates the immune system, such as a cytokine, an adjuvant and an immune checkpoint inhibitor.

“Immune checkpoint inhibitor” refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include PD-1/PD-L1 and CTLA-4/B7-1/B7-2. Some immune checkpoint inhibitors are used to treat cancer.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, “combination therapy” refers to a treatment in which a subject is given two or more therapeutic agents, such as at least two or at least three therapeutic agents, for treating a single disease. In some embodiments, each therapy can result in an independent pharmaceutical effect, and together can result in an additive or synergistic pharmaceutical effect.

As used herein, “treating” a subject with a disease or condition means that the subject's symptoms are partially or totally alleviated or remain static following treatment. Hence treating encompasses prophylaxis, therapy and/or cure. Prophylaxis refers to prevention of a potential disease and/or a prevention of worsening of symptoms or progression of a disease.

As used herein, “treatment” means any manner in which the symptoms of a condition, disorder or disease or other indication, are ameliorated or otherwise beneficially altered.

As used herein, “therapeutic effect” means an effect resulting from treatment of a subject that alters, typically improves or ameliorates the symptoms of a disease or condition or that cures a disease or condition.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, the term “subject” refers to an animal, including a mammal, such as a human being.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, a “tumor” refers to an abnormal mass of tissue that results when cells divide more than they should or do not die when they should. Tumors may be benign (not cancer), or malignant (cancer).

As used herein, a “lesion” refers to an area of abnormal tissue. A lesion may be benign (not cancer) or malignant (cancer).

As used herein, an “anti-cancer agent” refers to any molecules that are used for treatment to stop or prevent cancer. Examples may include, but are not limited to, small chemical molecules, antibodies, antibody conjugates, immunomodulators, or any combination thereof.

As used herein, a “suppressor cell” or an “immunosuppressor cell” refers to cells that are able to decrease or inhibit the function of immune effector cells such as CD8+ T effector cells. Example for suppressor cells may include, but are not limited to, regulatory T cells, M2 macrophages, myeloid derived suppressor cells, tumor associated fibroblasts, or cancer associated fibroblasts.

As used herein, an “immunosuppressive agent” refers to an agent that decreases the body's immune responses. It reduces the body's ability to fight infections and other diseases, such as cancer.

As used herein, “resistant to treatment” refers to that a disease or a pathological condition that is not responsive or exhibits insufficient efficacy to a treatment, so that this treatment is not effective or does not show efficacy, or is reduced in efficacy compared to a desirable level, in treating the disease or pathological condition.

As used herein, “systemic immune response” refers to the ability of a subject's immune system to respond to an immunologic challenge or immunologic challenges, including those associated with a tumor, a lesion or a cancer, in a systemic manner. Systemic immune response can include systemic response of the subject's adaptive immune system and/or innate immune system. Systemic immune response includes an immune response across different tissues, including the blood stream, lymph node, bone marrow, spleen and/or the tumor microenvironment, and in some cases, includes a coordinated response among the tissues and organs and various cells and factors of the tissues and organs.

As used herein, “local immune response” refers to the immune response in a tissue or an organ to an immunologic challenge or immunologic challenges including those associated with a tumor, a lesion or a cancer. Local immune response can include the adaptive immune system and/or innate immune system. Local immunity includes immune response concurrently occurring at different tissues, including the blood stream, lymph node, bone marrow, spleen and/or the tumor microenvironment.

### VII. EXEMPLARY EMBODIMENTS

Among the provided embodiments are:

1. A method of treating a tumor or a lesion in a subject by activating an immune cell response comprising:

(a) administering to a subject having a tumor or a lesion a conjugate comprising a phthalocyanine dye linked to a targeting molecule that binds to PD-L1; and

(b) illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located;

wherein the method results in the killing of the PD-L1 expressing immune cell and thereby inhibits the growth of the tumor or the lesion.

2. A method of treating a subject having a low response or that is unresponsive to a prior immunotherapy for a tumor or a lesion comprising:

(a) identifying a subject having a low response or that is unresponsive to a prior immunotherapy for a tumor or a lesion;

(b) administering to a subject having a tumor or a lesion that has had a low response or that was unresponsive to a prior immunotherapy a conjugate comprising a phthalocyanine dye linked to a targeting molecule that binds to PD-L1; and

(c) illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where PD-L1 expressing cells are located;

wherein the method results in the killing of a PD-L1 expressing cell and thereby increases the number or activity of immune cells in the tumor and/or in the tumor microenvironment.

3. The method of embodiment 2, wherein the PD-L1 expressing cell is an immune cell.

4. A method of enhancing a response to an anti-cancer agent in a subject having a tumor or a lesion comprising:

(a) administering an anti-cancer agent to a subject having a tumor or a lesion;

(b) administering a conjugate comprising a phthalocyanine dye linked to a targeting molecule that binds to PD-L1 to the subject; and

(c) illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located;

wherein the method results in a greater inhibition of growth of the tumor or the lesion compared to the inhibition by treatment with the anti-cancer agent alone.

5. A method of enhancing a response to an anti-cancer agent in a subject having a tumor or a lesion comprising:

administering to a subject a conjugate comprising a phthalocyanine dye linked to a targeting molecule that binds to PD-L1 to the subject; and illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, wherein the subject had been administered an anti-cancer agent, and

wherein the method results in a greater inhibition of growth of the tumor or the lesion compared to the inhibition by treatment with the anti-cancer agent alone.

6. A method of enhancing a response to an anti-cancer agent in a subject having a tumor or a lesion comprising:

administering an anti-cancer agent to a subject; wherein the subject had received a treatment comprising administering a conjugate comprising a phthalocyanine dye linked to a targeting molecule that binds to PD-L1 to the subject; and illuminating, at a wavelength of at or about 600 nm to at or about 850 nm and at a dose of from at or about 25 J/cm² to at or about 400 J/cm² or from at or about 2 J/cm fiber length to at or about 500 J/cm fiber length, a target area where a PD-L1 expressing immune cell is located, and

wherein the method results in a greater inhibition of growth of the tumor or the lesion compared to the inhibition by treatment with the anti-cancer agent alone.

7. The method of any of embodiments 4-6, wherein the anti-cancer agent is selected from a checkpoint inhibitor, an immune adjuvant, a chemotherapeutic agent, radiation, and a biologic comprising an anti-cancer targeting molecule that binds to a tumor cell.

8. The method of any of embodiments 4-7, wherein the anti-cancer agent is an antibody conjugate.

9. The method of embodiments 7 or 8, wherein the antibody conjugate comprises a phthalocyanine dye, a toxin, or a TLR agonist.

10. A method of vaccinating a subject to generate an anti-cancer immune response, comprising:

(a) administering a conjugate comprising a phthalocyanine dye linked to a targeting molecule that binds to PD-L1 to a subject; and

(b) illuminating a target area;

wherein the method results in an anti-cancer response selected from a delay or inhibition in the appearance of or growth of a tumor in the subject or an appearance or increase in T memory cells in the vicinity of a tumor.

11. The method of embodiment 10, wherein the illuminating is carried out at a wavelength of at or about 600 nm to at or about 850 nm and at a dose of from at or about 25 J/cm² to at or about 400 J/cm² or from at or about 2 J/cm fiber length to at or about 500 J/cm fiber length.

12. The method of embodiments 10 or 11, wherein the target area comprises PD-L1 expressing cells.

13. The method of embodiments 12, wherein the PD-L1 expressing cell is an immune cell.

14. The method of any of embodiments 1-13, wherein the method results in the killing of the PD-L1 expressing cell or the PD-L1 expressing immune cell.

15. The method of any of embodiments 1-14, wherein the subject is administered the PD-L1 conjugate to treat and/or inhibit the growth of a first tumor or a first lesion; and the method inhibits or delays the appearance of one or more second tumors or lesions or a metastasis of the first tumor or the first lesion.

16. The method of embodiment 15, wherein the one or more second tumor is phenotypically and/or genotypically different from the first tumor.

17. The method of embodiments 15 or 16, wherein the one or more second tumor is not derived from a metastasis of the first tumor.

18. The method of any of embodiments 1-17, wherein the treatment delays regrowth of the tumor or the lesion, prevents a relapse of a cancer or prolongs the duration of remission of a cancer.

19. The method of any of embodiments 1-18, wherein the PD-L1 expressing immune cell is selected from the group consisting of monocytes, macrophages, dendritic cells (DC), M2 tumor associated macrophages (M2 TAM), tolerogenic dendritic cells (tDC) and myeloid derived suppressor cells (MDSC).

20. The method of any of embodiments 1-19, wherein the PD-L1 expressing immune cell is located in the tumor, the tumor microenvironment or a lymph node.

21. The method of any of embodiments 1-20, wherein the tumor or the lesion comprises PD-L1 negative tumor cells.

22. The method of embodiments 21, wherein more than at or about 40%, 50%, 60%, 70%, 80%, 90% or 95% of the tumor cells in the tumor or the lesion are PD-L1 negative tumor cells.

23. The method of any of embodiments 1-22, wherein the inhibition of the growth of the tumor or the lesion and/or killing of the PD-L1 expressing cell is dependent on the presence of CD8+ T cells.

24. The method of any of embodiments 1-23, wherein the subject has been previously treated with an anti-cancer treatment and/or an immune checkpoint inhibitor.

25. The method of any of embodiments 1-24, wherein the subject has been previously treated with an immune checkpoint inhibitor.

26. The method of embodiments 24 or 25, wherein the subject has failed or has relapsed after the previous treatment with the anti-cancer treatment and/or immune checkpoint inhibitor.

27. The method of any of embodiments 24-26, wherein the subject has failed or has relapsed after the previous treatment with the immune checkpoint inhibitor.

28. The method of any of embodiments 24-27, wherein the inhibition of tumor growth resulting from carrying out the method is greater compared to the inhibition of tumor growth as a result of the previous treatment with the anti-cancer treatment and/or immune checkpoint inhibitor.

29. The method of any of embodiments 24-28, wherein the inhibition of tumor growth resulting from carrying out the method is greater compared to the inhibition of tumor growth as a result of the previous treatment with the immune checkpoint inhibitor.

30. The method of any of embodiments 1-29, wherein prior to the administering, the subject has a tumor or a lesion having a low level of CD8+ T cell infiltration.

31. The method of any of embodiments 1-30, wherein the number, level or activity of immune cells is increased in the tumor or in the tumor microenvironment after the administering and the illuminating.

32. The method of embodiments 1-31, wherein the number or level of CD8+ T cell infiltration in the tumor or the lesion is increased after the administering and the illuminating.

33. The method of embodiments 1-32, wherein the number of memory T cells in the vicinity of the tumor is increased after the administering and the illuminating.

34. A method of enhancing an innate immune response in a subject having a tumor or a lesion, the method comprising:

(a) administering a conjugate comprising a phthalocyanine dye linked to a targeting molecule that binds to PD-L1 to the subject; and

(b) illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length;

thereby enhancing the innate immune response in the subject.

35. The method of embodiment 34, wherein enhancing the innate immune response comprises an increase in activated dendritic cells (DC) or antigen-presenting dendritic cells.

36. The method of embodiment 35, wherein the activated DCs exhibit a cell surface phenotype of CD80+ and/or CD40+.

37. The method of embodiment 35, wherein the antigen-presenting dendritic cells exhibit a cell surface phenotype of CD11b+CD103+ CD11c+.

38. A method of increasing the number or level of immune cells in a tumor or a lesion, the method comprising:

(a) administering a conjugate comprising a phthalocyanine dye linked to a targeting molecule that binds to PD-L1 to a subject having a tumor or lesion; and

(b) illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length;

thereby increasing the number or level of immune cells in the tumor or lesion in the subject.

39. The method of embodiment 38, wherein the immune cell is an intratumoral neutrophil.

40. The method of embodiment 39, wherein the intratumoral neutrophil exhibits a cell surface phenotype of CD11b⁺ Ly6C^{-/low} Ly6G⁺

41. The method of embodiment 38, wherein the immune cell is an intratumoral effector T cell.

42. The method of embodiment 41, wherein the intratumoral effector T cell exhibits a cell surface phenotype of CD3+ CD8+ PD-1⁻.

43. A method of treating a heterogeneous tumor or lesion, the method comprising:

(a) administering a conjugate comprising a phthalocyanine dye linked to a targeting molecule that binds to PD-L1 to a subject having a tumor or lesion; and

(b) illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length;

thereby treating the heterogeneous tumor or lesion in the subject.

44. The method of embodiment 43, wherein the heterogeneous tumor or lesion comprises a plurality of different types of tumor cells or tumor cells from a plurality of different origins.

45. A method of treating an immunosuppressive tumor or lesion, the method comprising:

(a) administering a conjugate comprising a phthalocyanine dye linked to a targeting molecule that binds to PD-L1 to a subject having a tumor or lesion; and

(b) illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length;

thereby treating the immunosuppressive tumor or lesion in the subject.

46. The method of embodiment 45, wherein the immunosuppressive tumor or lesion comprises a tumor cell that expresses an immune checkpoint protein.

47. The method of embodiment 46, wherein the immune checkpoint protein is PD-L1, PD-1 or CTLA-4.

48. A method of treating a tumor or lesion, the method comprising:

(a) administering a conjugate comprising a phthalocyanine dye linked to a targeting molecule that binds to PD-L1 to a subject having a tumor or lesion comprising a tumor cell that is reduced in susceptibility to treatment with an immune checkpoint inhibitor; and

(b) illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length;

wherein after the illumination, the growth, size or viability of the tumor or lesion is reduced or inhibited.

49. A method of treating a tumor or lesion, the method comprising:

(a) administering a conjugate comprising a phthalocyanine dye linked to a targeting molecule that binds to PD-L1 to a subject having a tumor or lesion that has had a low response to, was unresponsive to, was resistant to, was refractory to, had failed to respond to or has relapsed after, a prior immunotherapy; and

(b) illuminating a target area where the tumor or lesion is located, at a wavelength of at or about 600 nm to at or about 850 nm and at a dose of from at or about 25 J/cm² to at or about 400 J/cm² or from at or about 2 J/cm fiber length to at or about 500 J/cm fiber length;

wherein the method results in the killing of a PD-L1 expressing cell in the target area.

50. The method of any of embodiments 2, 3, 14-33, 48 and 49, wherein the prior immunotherapy is a treatment with an immune checkpoint inhibitor.

51. The method of any of embodiments 2, 3, 14-33 and 48-50, wherein the subject has primary resistance or acquired resistance to a prior immunotherapy that comprises a PD-1/PD-L1 blockade therapy.

52. A method of treating a tumor or lesion, the method comprising:

(a) administering a conjugate comprising a phthalocyanine dye linked to a targeting molecule that binds to PD-L1 to a subject that is treatment-naïve for an immune checkpoint inhibitor or that has not previously received a treatment with an immune checkpoint inhibitor; and

(b) illuminating a target area where a tumor or lesion is located in the subject at a wavelength of at or about 600 nm to at or about 850 nm and at a dose of from at or about 25 J/cm² to at or about 400 J/cm² or from at or about 2 J/cm fiber length to at or about 500 J/cm fiber length; wherein after the illumination, the growth, size or viability of the tumor or lesion is reduced or inhibited.

53. The method of any of embodiments 15-33 and 48-52, wherein the subject is administered the conjugate to treat, inhibit the growth of and/or reduce the size of a first tumor or lesion; and the method inhibits, delays or prevents the appearance, growth or establishment of one or more second tumors or lesions, located distally to the first tumor or lesion.

54. A method of immunizing a subject having a first tumor or lesion, the method comprising:

(a) administering a conjugate comprising a phthalocyanine dye linked to a targeting molecule that binds to PD-L1 to a subject having a tumor or lesion; and

(b) illuminating a target area within the first tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length;

wherein the first tumor or lesion is inhibited in growth and/or reduced in size; and the appearance, growth or establishment of one or more second tumors or lesions, located distally to the treated first tumor or lesion, is inhibited, delayed or prevented.

55. The method of any of embodiments 15-33, 53 and 54, wherein the second tumor or lesion is a metastasis of the first tumor or lesion.

56. The method of any of embodiments 15-33 and 53-55, wherein the method results in killing of a PD-L1-expressing cell in the vicinity of the first tumor or lesion and/or activates an immune cell response, thereby inhibiting, delaying or preventing the appearance, growth or establishment of the second tumor or lesion.

57. The method of any of embodiments 15-33 and 53-56, wherein the second tumor or lesion is phenotypically and/or genotypically the same as the first tumor or lesion.

58. The method of any of embodiments 15-33 and 53-56, wherein the second tumor or lesion is phenotypically and/or genotypically different from the first tumor or lesion.

59. The method of any of embodiments 15-33, 53 and 54, wherein the second tumor or lesion is not derived from a metastasis of the first tumor or lesion.

60. The method of any of embodiments 1-59, wherein the method results in the killing of the PD-L1-expressing cell or the PD-L1-expressing immune cell.

61. The method of any of embodiments 1-60, wherein the tumor or lesion comprises a tumor cell, and the tumor cell does not express or has a reduced expression of an immune checkpoint protein.

62. The method of embodiment 61, wherein the immune checkpoint protein is selected from among PD-L1, PD-1, and CTLA-4.

63. The method of any of embodiments 7-62, wherein the tumor cell does not express PD-L1 in response to an inflammatory stimulus.

64. The method of embodiment 63, wherein the inflammatory stimulus is interferon.

65. The method of any of embodiments 7-64, wherein the tumor cell is not specifically recognized by an anti-PD-L1 antibody.

66. The method of any of embodiments 1-65, wherein the tumor or lesion comprises PD-L1 negative tumor cells.

67. The method of embodiment 66, wherein at least or at least about 40%, 50%, 60%, 70%, 80%, 90% or 95% of the tumor cells in the tumor or lesion are PD-L1 negative tumor cells.

68. The method of any of embodiments 1-67, wherein the treatment delays regrowth of the tumor or lesion, prevents a relapse of a cancer associated with the tumor or lesion or prolongs the duration of remission of a cancer associated with the tumor or lesion.

69. The method of any of embodiments 1-68, wherein the inhibition of the growth of the tumor or lesion and/or killing of the PD-L1-expressing cell is dependent on the presence of CD8+ T cells.

70. The method of any of embodiments 1-69, wherein the subject is naïve to treatment with an immune checkpoint inhibitor or has not previously received treatment with an immune checkpoint inhibitor.

71. The method of any of embodiments 1-69, wherein the subject has been previously treated with an immune checkpoint inhibitor.

72. The method of embodiment 71, wherein the subject has had a low response to, was unresponsive to, was resistant to, was refractory to, had failed to respond to or has relapsed after the previous treatment with the immune checkpoint inhibitor.

73. The method of embodiment 71 or 72, wherein the inhibition of the growth, size or viability of the tumor or lesion resulting from carrying out the method is greater compared to the inhibition resulting from the previous treatment with the immune checkpoint inhibitor.

74. The method of any of embodiments 71-73, wherein the immune checkpoint inhibitor is an inhibitor of PD-L1, PD-1 or CTLA-4.

75. The method of any of embodiments 24-74, wherein immune checkpoint inhibitor is a PD-1 inhibitor.

76. The method of embodiment 75, wherein the PD-1 inhibitor is an anti-PD-1 antibody.

77. The method of any of embodiments 24-74, wherein the immune checkpoint inhibitor is PD-L1 inhibitor.

78. The method of embodiment 77, wherein the PD-L1 inhibitor is an anti-PD-L1 antibody.

79. The method of any of embodiments 1-78, wherein the method increases the number or activity of immune cells in the tumor or lesion and/or in the microenvironment of the tumor or lesion.

80. The method of any of embodiments 1-79, wherein the target area comprises immune cells expressing PD-L1.

81. The method of any of embodiments 2-79, wherein the PD-L1 expressing cell is an immune cell.

82. The method of any of embodiments 1-81, wherein the immune cell is selected from the group consisting of monocytes, macrophages, dendritic cells (DC), M2 tumor associated macrophages (M2 TAM), tolerogenic dendritic cells (tDC) and myeloid derived suppressor cells (MDSC).

83. The method of any of embodiments 1-82, wherein the immune cell is located in the tumor, the tumor microenvironment or a lymph node.

84. The method of any of embodiments 1-83, wherein prior to administering the conjugate, the subject has a tumor or lesion having a low number or level of CD8+ T cell infiltration.

85. The method of any of embodiments 1-84, wherein the number, level or activity of immune cells is increased in the tumor or lesion or in the microenvironment of the tumor or lesion after the administering and the illuminating.

86. The method of embodiment 84 or 85, wherein the number or level of CD8+ T cell infiltration in the tumor or lesion is increased after the administering and the illuminating.

87. The method of any of embodiments 84-86, wherein the number or level of memory T cells in the vicinity of the tumor or lesion is increased after the administering and the illuminating.

88. The method of any of embodiments 1-87, wherein the method enhances the innate immune response in the subject.

89. The method of embodiment 88, wherein the enhancing the innate immune response comprises an increase in activated dendritic cells (DC) or antigen-presenting dendritic cells.

90. The method of embodiment 89, wherein the activated DCs exhibit a cell surface phenotype of CD80+ and/or CD40+.

91. The method of embodiment 89, wherein the antigen-presenting dendritic cells exhibit a cell surface phenotype of CD11b+CD103+ CD11c+.

92. The method of any of embodiments 1-91, wherein the method increases the number or level of immune cells in the tumor or lesion in the subject.

93. The method of embodiment 92, wherein the immune cell is an intratumoral neutrophil.

94. The method of embodiment 93, wherein the intratumoral neutrophil exhibits a cell surface phenotype of CD11b⁺ Ly6C^{-/low} Ly6G⁺

95. The method of embodiment 92, wherein the immune cell is an intratumoral effector T cell.

96. The method of embodiment 95, wherein the intratumoral effector T cell exhibits a cell surface phenotype of CD3⁺ CD8⁺ PD-1⁻.

97. The method of any of embodiments 1-96, wherein the method treats a heterogeneous tumor or lesion in the subject.

98. The method of embodiment 97, wherein the heterogeneous tumor or lesion comprises a plurality of different types of tumor cells or tumor cells from a plurality of different origins.

99. The method of any of embodiments 1-98, wherein the method treats an immunosuppressive tumor or lesion in the subject.

100. The method of embodiment 99, wherein the immunosuppressive tumor or lesion comprises a tumor cell that expresses an immune checkpoint protein.

101. The method of embodiment 100, wherein the immune checkpoint protein is PD-L1, PD-1 or CTLA-4.

102. The method of any of embodiments 1-101, wherein the targeting molecule is or comprises an antibody, an antigen-binding antibody fragment or antibody-like molecule that binds PD-L1.

103. The method of embodiment 102, wherein the targeting molecule is or comprises an anti-PD-L1 antibody or antigen-binding fragment thereof.

104. The method of embodiment 103, wherein the antibody or antigen-binding fragment comprises complementary determining regions (CDRs) from an antibody selected from the group consisting of atezolizumab (MPDL3280A, Tecentriq, RG7446), avelumab (Bavencio), BCD-135, BGB-A333, BMS-936559 (MDX-1105), CBT-502 (TQB-2450), cosibelimab (CK-301), CS1001 (WPB3155), durvalumab (MEDI4736, Imfinzi), FAZ053, HLX20, INBRX-105, KN035, KN046, LDP, LY3300054, LY3415244, M7824 (MSB0011359C), MCLA-145, MSB2311, NM-01, REGN3504, SHR-1316 (HTI-1088), STI-3031 (IMC-001, STI-A1015), TG-1501, and ZKAB001 (STI-A1014).

105. The method of embodiment 103 or 104, wherein the antibody or antigen-binding fragment comprises complementary determining regions (CDRs) from atezolizumab, avelumab, durvalumab, KN035 or CK-301.

106. The method of any of embodiments 103-105, wherein the antibody or antigen-binding fragment is selected from the group consisting of atezolizumab, avelumab, durvalumab, KN035, and CK-301, or a biosimilar, interchangeable, biobetter, copy biological or biogeneric thereof, or an antigen-binding fragment thereof.

107. The method of any of embodiments 103-106, wherein the antibody or antigen-binding fragment is selected from the group consisting of atezolizumab, avelumab, durvalumab, KN035, and CK-301.

108. The method of any of embodiments 1-107, wherein the target area is a lymph node or in the vicinity of a lymph node.

109. The method of any of embodiments 1-108, wherein the subject exhibits a durable response, prolonged progression-free survival, a reduced chance of relapse, and/or a reduced chance of metastasis, after the administering and the illuminating.

110. The method of any of embodiments 1-109, wherein the phthalocyanine dye is a Si-phthalocyanine dye.

111. The method of embodiment 110, wherein the Si-phthalocyanine dye is IR700.

112. The method of any of embodiments 1-111, wherein the illuminating is carried out between 30 minutes and 96 hours after administering the conjugate.

113. The method of any of embodiments 1-112, wherein the illuminating is carried out 24 hours ±4 hours after administering the conjugate.

114. The method of any of embodiments 1-113, wherein the target area is illuminated at a wavelength of 690 ±40 nm.

115. The method of any of embodiments 1-114, wherein the target area is illuminated at a dose of at or about of 50 J/cm² or at or about 100 J/cm of fiber length.

116. The method of any of embodiments 1-115, wherein the tumor or lesion is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

117. The method of any of embodiments 1-116, wherein one or more of steps of the method are repeated.

118. The method of embodiment 117, wherein the administration of the conjugate is repeated one or more times, optionally wherein after each repeated administration of the conjugate, the illuminating step is repeated.

119. The method of any of embodiments 1-118, further comprising administering an additional therapeutic agent or anti-cancer treatment.

### VIII. EXAMPLES

The following examples are included for illustrative purposes only and are not intended to limit the scope of the invention.

### Example 1: Generation of Anti-PD-L1 Antibody-IRDye 700 Conjugate

This example describes a method for preparing a conjugate containing IRDye 700DX (IR700) linked to the anti-PD-L1 antibody 10F.9G2, thus producing a 10F.9G2-IRDye 700DX (anti-PD-L1-IR700 or α-PD-L1-IR700 conjugate).

10F.9G2 monoclonal antibody (mAb) was buffer exchanged into lx PBS pH 7.1 then concentrated to 8.2 mg/mL. The mAb (16 mg) was diluted to 3 mg/mL with 100 mM sodium phosphate pH 8.6 to achieve a target pH 8.0-8.5. IR700 NHS ester (1 mg, IR700; LI-COR Bioscience, Lincoln, NE) was solubilized into DMSO at a concentration of 10 g/L. The solubilized dye was then added to the mAb for a target dye to mAb ratio of 1 mg IR700 NHS ester to 16 mg of mAb. Conjugation was held for 2 hours at RT. The reaction was quenched by the addition of 1 M glycine to a target batch concentration of 20 mM glycine. The quench was held for 1 hour at RT. Buffer exchange was performed using Millipore 30 kDa molecular weight cut-off Amicon centrifuge filters by concentration and dilution of up to 3 cycles at ˜3000 RPM.

The mixture was purified using a Sephadex G50 column (PD-10; GE Healthcare, Piscataway, N.J.). Protein concentration was determined with Coomassie Plus protein assay kit (Pierce Biotechnology, Rockford, Ill.) by measuring the absorption at 595 nm with a UV-Vis system (8453 Value System; Agilent Technologies, Palo Alto, Calif.). The concentration of IR700 was measured by absorption with the UV-Vis system to confirm the number of fluorophore molecules conjugated to each anti-PD-L1 antibody molecule. The number of IR700 per antibody was about 3.

Purity of the anti-PD-L1-IR700 conjugate was confirmed by analytical size-exclusion HPLC (SE-HPLC). SE-HPLC was performed using an Agilent 1100 HPLC system (Santa Clara, Calif.) equipped with a PDA detector controlled by Chemstation software. SE chromatography was performed on a Shodex KW-803 column (New Yok, N.Y.) eluted for 20 minutes using phosphate buffered saline (PBS) at 1.0 mL/min. The anti-PD-L1-IR700 preparation exhibited strong association and contained no detectable mAb aggregates as determined by SE-HPLC.

To determine the in vitro binding characteristics of IR700 conjugates, ¹²⁵I labeling of the conjugates using the Indo-Gen procedure was performed. Minimal loss of mAb with IR700 conjugation was observed. Immunoreactivity assay was performed as described previously. Briefly, after trypsinization, 2×10⁶ of tumor cells were resuspended in PBS containing 1% bovine serum albumin (BSA). ¹²⁵1-anti-PD-L1-IR700 (1 mCi, 0.2 μg) was added and incubated for 1 h on ice. The cells were washed, pelleted, the supernatant was decanted, and the cells were counted in a 2470 Wizard gamma-counter (Perkin Elmer, Shelton, Conn.). Nonspecific binding to the cells was examined under conditions of excess unlabeled antibody (200 μg of non-labeled antibody).

### Example 2: Anti-PD-L1-IR700 PIT Inhibits the Growth of CT26 Tumors

This example describes the activity of anti-PD-L1 antibody-IR700 conjugate with or without light illumination on primary tumors.

BALB/c mice at the age of 6-8 weeks were inoculated with 1×10⁶ CT26 murine colon carcinoma cells subcutaneously in the right hind flank. When allograft tumors grew to a size of about 150 mm³ (approximately day 6 after tumor implantation) mice were administered with saline (100 μL; control) or anti-PD-L1-IR700 conjugate (100 μg) generated substantially as described in Example 1 above. Twenty-four hours after administration of the conjugate, tumors in the photoimmunotherapy (PIT) group were illuminated at 690 nm and a dosage of 75, 100 or 150 J/cm². The tumor growth was observed over 24 days, and tumor volume was calculated using the formula: tumor volume=(width×width)×length/2.

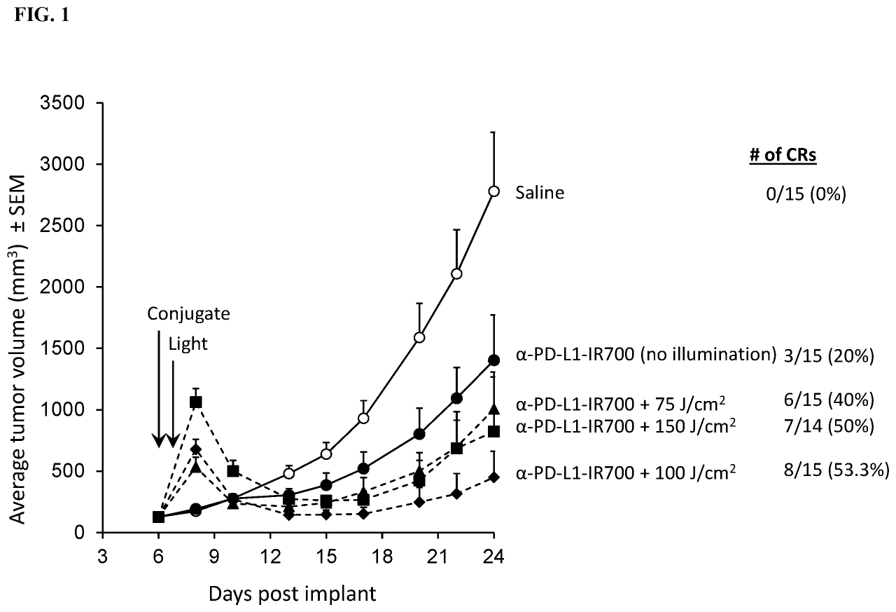

In mice that received the anti-PD-L1-IR700 (α-PD-L1-IR700) combined with illumination (PIT), the growth of tumors was substantially inhibited in comparison to tumor growth inhibition in control mice that received saline or anti-PD-L1-IR700 conjugate alone without PIT (FIG. 1; dashed lines (PIT) vs. solid lines (controls)). Mice receiving anti-PD-L1-IR700 conjugate alone without PIT also exhibited a moderate reduction in tumor growth compared to saline control mice (FIG. 1; open circles vs. closed circles).

In addition to examining the tumor growth, the rate of complete response (CR) was compared among the treatment groups. In this example, CR was defined as tumors with a volume of less than 100 mm³ sustained for at least 2 weeks. At 24 days post tumor implantation, at least 50% of mice treated with anti-PD-L1-IR700 PIT at 100 and 150 J/cm² achieved CR, while none of the animals in the saline control group achieved CR. The anti-PD-L1-IR700 PIT treated groups achieved greater numbers of CR than in mice receiving anti-PD-L1-IR700 conjugate alone without PIT, only 20% of which achieved a CR (FIG. 1). The results showed that anti-PD-L1-IR700 PIT treatment results in substantial inhibition of growth of primary tumors, with more than half of the mice achieving a CR when receiving illumination at certain light doses.

### Example 3: Anti-PD-L1-IR700 PIT Inhibits Tumor Growth in Mice Challenged with a Second CT26 Tumor

This example describes the effect of a prior anti-PD-L1-IR700 conjugate administration and PIT in animals challenged with a second tumor of the same tumor type after a successful inhibition of growth of a first tumor.

Mice that have achieved CR, from the treatment groups anti-PD-L1-conjugate with PIT treatment (at all three light dose levels) and anti-PD-L1-conjugate alone, from Example 2, were implanted subcutaneously on the contralateral flank with a second tumor of the same type (1×10⁶ CT26 murine colon carcinoma cells/mouse) on day 56 post initial tumor implantation. A subset of naïve mice (no previous treatment) were implanted in the same manner as a control group. The growth of the second tumor was observed over approximately 20 days, and tumor volume was calculated using the formula: tumor volume=(width×width)×length/2.

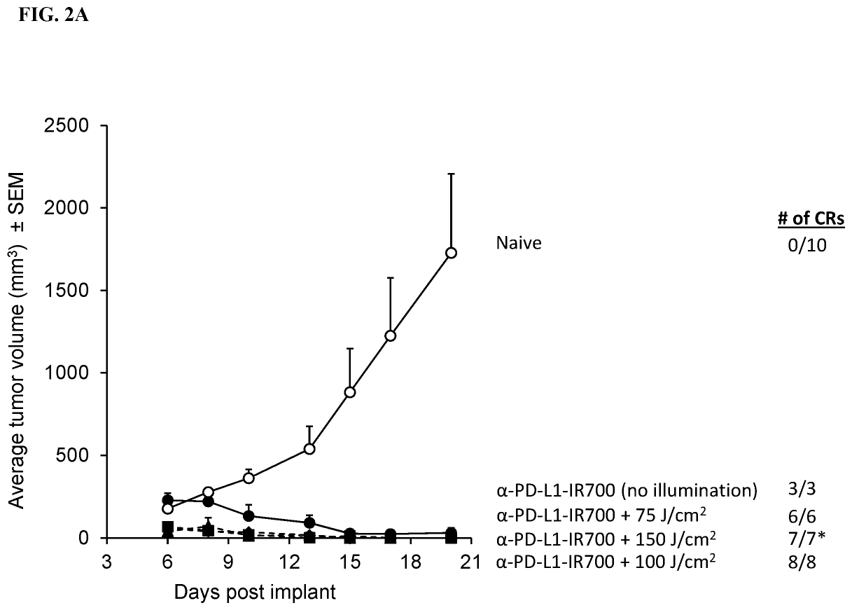

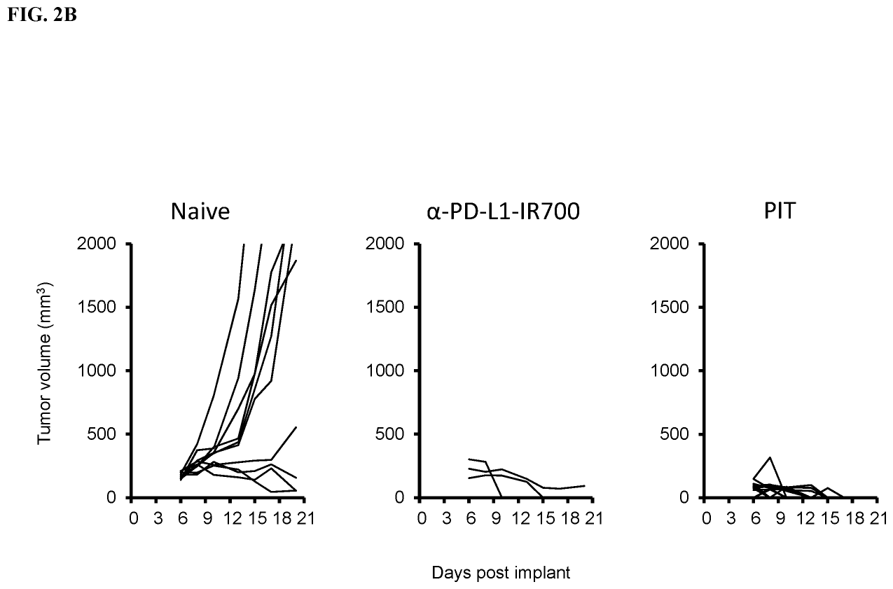

In mice that previously received the anti-PD-L1-IR700 PIT and then were re-challenged with a second CT26 implantation on the contralateral flank, the growth of tumors was substantially inhibited in comparison to tumor growth inhibition in naïve control mice (FIG. 2A depicting average tumor volume; FIG. 2B depicting individual mice).

In addition to examining the tumor growth, the rate of complete response (CR) was compared among the treatment groups. At 21 days post second tumor implantation, 100% of the animals previously treated with the anti-PD-L1-IR700 conjugate (with or without previous PIT treatment) achieved a CR. In contrast, none of the naïve control mice achieved a CR. The results showed that mice previously treated with an anti-PD-L1-IR700 conjugate (with or without light illumination for PIT) successfully rejected a second tumor of the same type.

### Example 4: Anti-PD-L1-IR700 PIT Inhibits the Growth in Mice Challenged with a Third Tumor of a Different Type

This example describes the effect of a prior anti-PD-L1-IR700 conjugate administration combined with PIT in animals challenged with a third tumor of a different tumor type, after a successful inhibition of growth of a first tumor and the rejection of a second tumor of the same type as the first tumor.

Mice that have achieved CR, from the treatment groups anti-PD-L1-conjugate with PIT treatment (at all three light dose levels; *except one CR mouse from the α-PD-L1-IR700+100 J/cm² group in FIG. 2A) and anti-PD-L1-conjugate alone, from Example 3, were implanted subcutaneously on day 104 post first tumor implantation with 3×10⁶ 4T1 mouse mammary gland cancer cells (a syngeneic BALB/c mouse tumor line derived from a different tissue than the CT26 tumor cell line) engineered to overexpress epithelial cell adhesion molecule (4T1-EpCAM). A subset of naïve mice (no previous treatment) were implanted in the same manner as a control group. The growth of the second tumor was observed over approximately 20 days, and tumor volume was calculated using the formula: tumor volume =(width x width) x height/2.

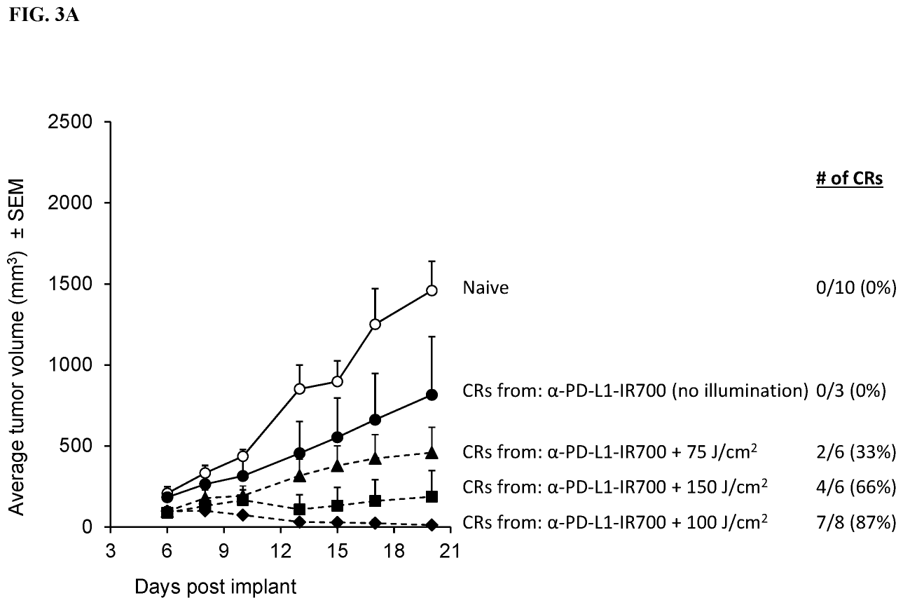

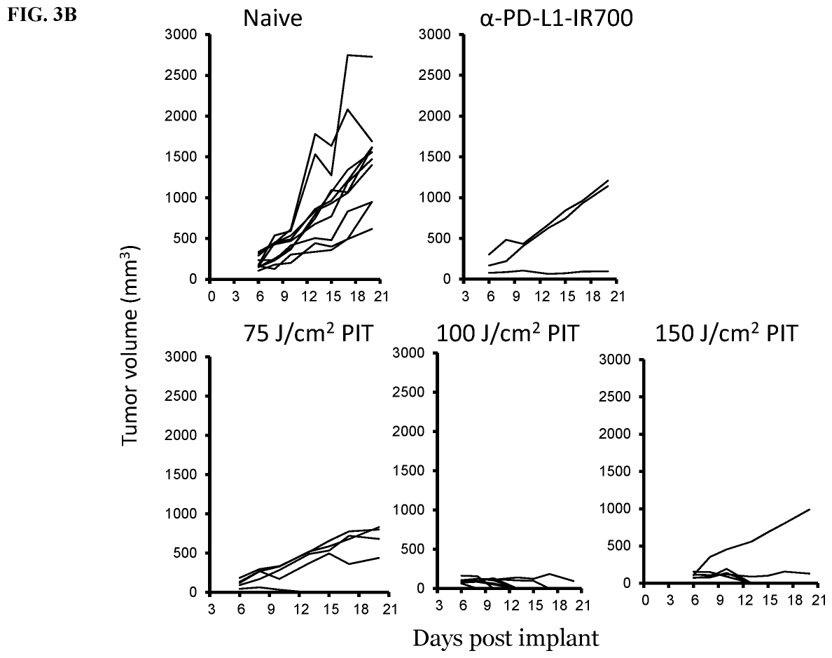

Surprisingly, in mice that previously received the anti-PD-L1-IR700 PIT, rejected a second tumor of the same type, and then were re-challenged with a 4T1-EpCAM tumor, the growth of tumors was inhibited compared to mice previously treated with an anti-PD-L1-IR700 conjugate only without light illumination or compared to naïve control mice (FIG. 3A depicting group average tumor volume; FIG. 3B depicting individual mice). Even more unexpectedly, greater than 50% of the mice in the group previously treated with anti-PD-L1-IR700 conjugate combined with 100 or 150 J/cm² PIT, achieved a CR (87% and 66%, respectively). The mice previously treated with anti-PD-L1 IR700 only without light illumination (no PIT treatment) and the naïve control group did not result in any CRs. The results showed that surprisingly, mice previously treated with an anti-PD-L1-IR700 conjugate with light illumination (PIT) successfully rejected inoculation of a tumor of a different type.

### Example 5: CD8 Cells are Required for PD-L1 PIT-mediated Tumor Rejection

This example describes that the effect of the anti-PD-L1-IR700 PIT on tumor growth in vivo is dependent on a functional CD8⁺ T cell population.

BALB/c mice were inoculated with 1×10⁶ CT26 cells per mouse subcutaneously on the right hind flank. For CD8⁺ T cell depletion, mice were administered anti-CD8a antibody (BioXCell, clone 2.43, catalog# BP0061) (100 μg/mouse) by intraperitoneal injection on days 6 and 9 post tumor cell inoculation. When allograft tumors grew to about 150 mm³ in size, mice were administered the anti-PD-L1-IR700 conjugate (100 μg) or a saline control. The anti-PD-L1-IR700 conjugate was administered at day 6 and twenty-four hours later, tumors on the right flanks in the mice of PIT group were illuminated at 690 nm at a dose of 100 J/cm².

As shown in FIG. 4, in immunocompetent mice treated with anti-PD-L1-IR700 conjugate with light illumination (PIT), the growth of the tumors was substantially inhibited in comparison to the control saline or anti-PD-L1-IR700 conjugate only without light illumination. Surprisingly, in mice depleted of CD8⁺ T cells, the tumor inhibitory effect of anti-PD-L1-IR700 PIT was completely abolished (FIG. 4), indicating the effect of the anti-PD-L1-IR700 PIT treatment was mediated by CD8⁺ T cells. In addition, the number of mice achieving CR in the group of mice treated with anti-PD-L1-IR700 PIT without CD8⁺ T cell depletion was substantially higher than in mice depleted of CD8⁺ T cells treated with anti-PD-L1-IR700 PIT as compared to mice where CD8⁺ T cells were not depleted (46.7% CR versus 6.7% CR, respectively). The results showed that CD8⁺ T cells are required for the tumor growth inhibition effect of the anti-PD-L1-IR700 PIT.

### Example 6: Anti-PD-L1-IR700 PIT Delays or Rejects Tumor Growth in Mice Challenged with Various Tumor Types

This example describes the effect of anti-PD-L1-IR700 conjugate administration and PIT in animals challenged with a second tumor of various types, after a successful inhibition of growth of a first tumor.

Round 1: BALB/c mice at age of 6-8 weeks were inoculated with 1×10⁶ CT26 cells/mouse subcutaneously on the right hind flank. When allograft tumors grew to a size about 150 mm³, mice were administered with anti-PD-L1-IR700 conjugate (100 μg). Twenty-four hours after administration of the conjugate, tumors were illuminated at 690 nm and a dosage of 100 J/cm². Mice treated with anti-PD-L1-IR700 PIT that achieved CR were collected and placed into 3 subgroups for a second round of tumor challenge.

Round 2: In each of the three subgroups (“CR” groups), the mice were implanted with a second tumor on the contralateral flank. Each of the three subgroups also had a matching control group of mice that had not been previously treated (“naïve” control groups) and were implanted with the same tumor cells as their matched subgroup. The three subgroups were implanted with a second tumor as follows: (a) CT26, (b) 4T1.WT (parental/wild-type 4T1 cells without engineering), and (c) RENCA mouse renal adenocarcinoma. The growth of the second tumor was observed over approximately 20 days, and tumor volume was calculated using the formula: tumor volume=(width×width)'height/2.

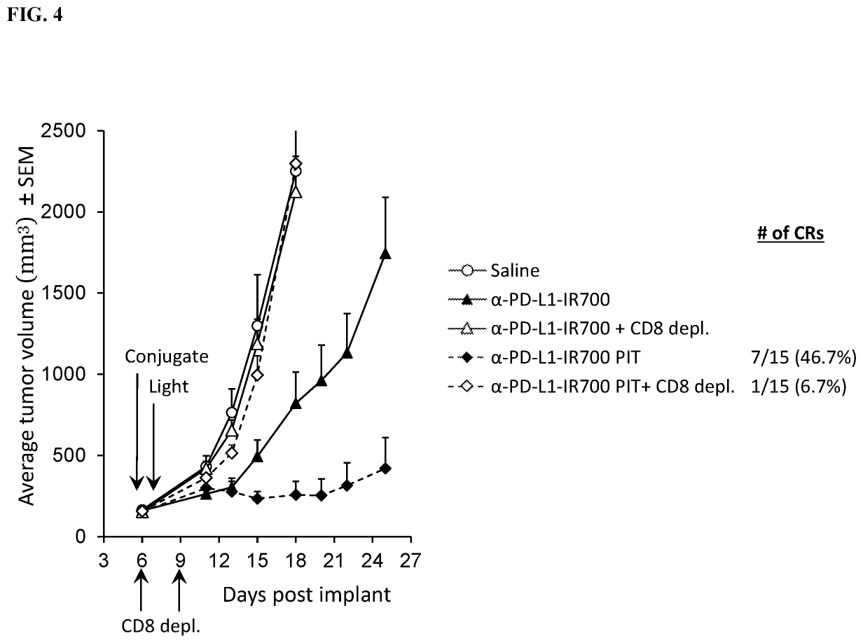

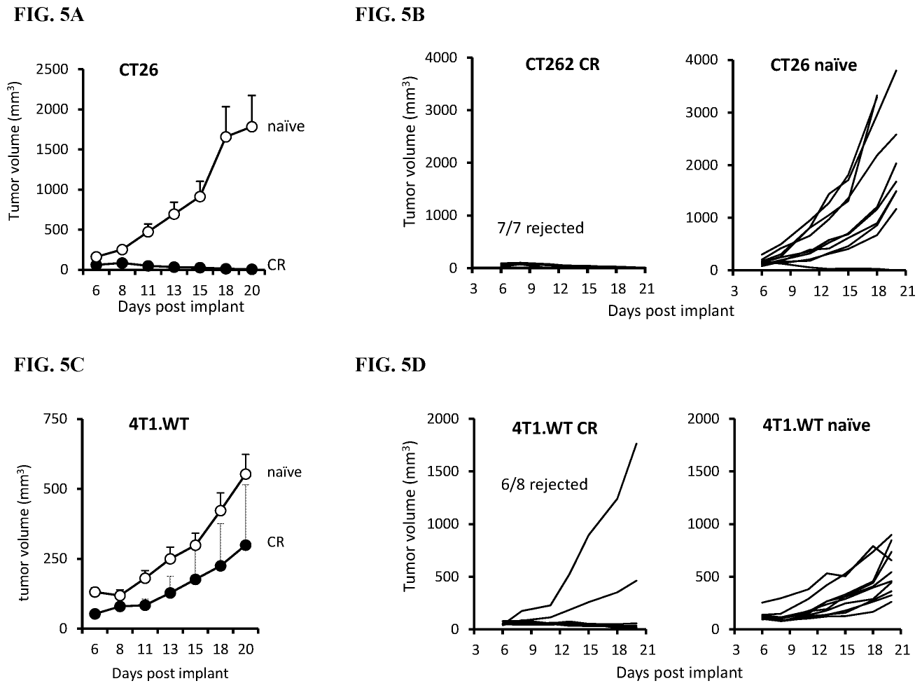

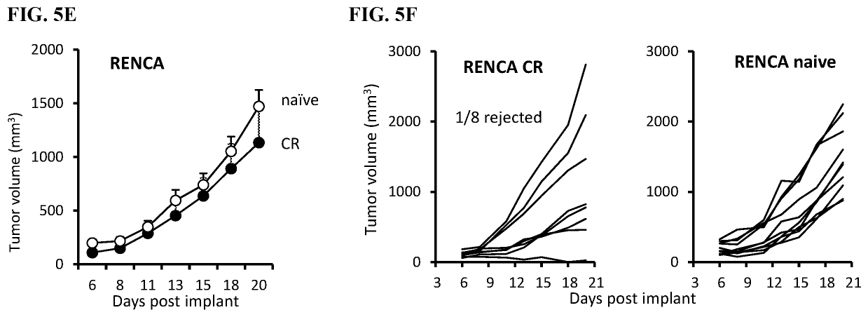

As shown in FIGS. 5A (group average) and 5B (individual mice), animals implanted with a CT26 tumor in Round 2 that had been previously treated in Round 1 with the anti-PD-L1-IR700 PIT exhibited substantial tumor growth inhibition in Round 2, with 100% (7/7) of the mice achieved CR after challenge with CT26 tumors in Round 2. In comparison, in the naïve control animals, tumor growth was not inhibited. As shown in FIGS. 5C (group average) and 5D (individual mice), animals implanted with a 4T1.WT tumor in Round 2 that had been previously treated in Round 1 with the anti-PD-L1-IR700 PIT exhibited substantial inhibition of tumor growth, with 6 of 8 treated animals achieving a CR. In contrast, none of the control naïve animals implanted in Round 2 with 4T1.WT tumors achieved a CR.

Animals implanted with RENCA (FIGS. 5E (group average) and 5F (individual mice)) in Round 2 that had been previously treated in Round 1 with the anti-PD-L1-IR700 PIT exhibited only a very modest inhibition of tumor growth as compared to the naïve control groups, since anti-PD-L1-IR700 PIT treatment resulted in only 1/8 CR in comparison to naïve animals that had 0/8 CR after RENCA inoculation. These results from the various tumor challenges after achieving CR showed that mice previously treated with an anti-PD-L1-IR700 conjugate and light illumination (PIT) successfully rejected a second tumor of the same type or certain different tumor types.

### Example 7: Anti-PD-L1-IR700 PIT Inhibits the Growth in Mice Challenged with a Third 4T1-EpCAM Tumor

Animals from the Round 2 subgroup that had been implanted with CT26 on the contralateral flank and exhibited CR from Example 6, were subjected to a Round 3 challenge.

Round 3: animals that achieved a CR from the CT26 group from Round 2 were implanted with a 4T1-EpCAM tumor on right axilla. As a control, naïve animals (not previously treated) were also implanted with 4T1-EpCAM tumor on right axilla. The growth of the third tumor was observed over approximately 21 days, and tumor volume was calculated using the formula: tumor volume=(width×width)×height/2.

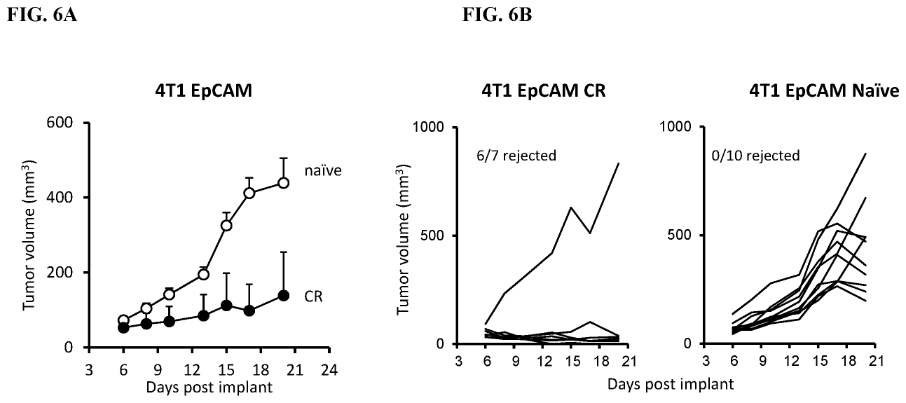

As shown in FIGS. 6A (group average) and 6B (individual mice), the animals exhibited substantial inhibition of the 4T1-EpCAM tumor in Round 3 treatment, and 6 of 7 animals exhibited a CR. In contrast, naïve animals exhibited greater tumor growth and no animals achieved a CR. The results showed that mice previously treated with an anti-PD-L1-IR700 conjugate and light illumination (PIT) successfully rejected a third tumor of a different type.

### Example 8: Anti-PD-L1-IR700 PIT Inhibits the Growth of CT26 Tumor Cells with PD-L1 Knock-out

This example describes the activity of anti-PD-L1 antibody-IR700 conjugate and light illumination (PIT) on tumor cells not expressing a PD-L1 due to a CRISPR-Cas9-mediated gene disruption (knock-out).

A genetic disruption at the CD274 gene (encoding PD-L1) was introduced to CT26 cells by clustered regularly interspaced short palindromic repeats (CRISPR)/CRISPR associated protein 9 (Cas9) using a guide RNA (gRNA) targeting PD-L1, to phenotypically knock out the expression of PD-L1.

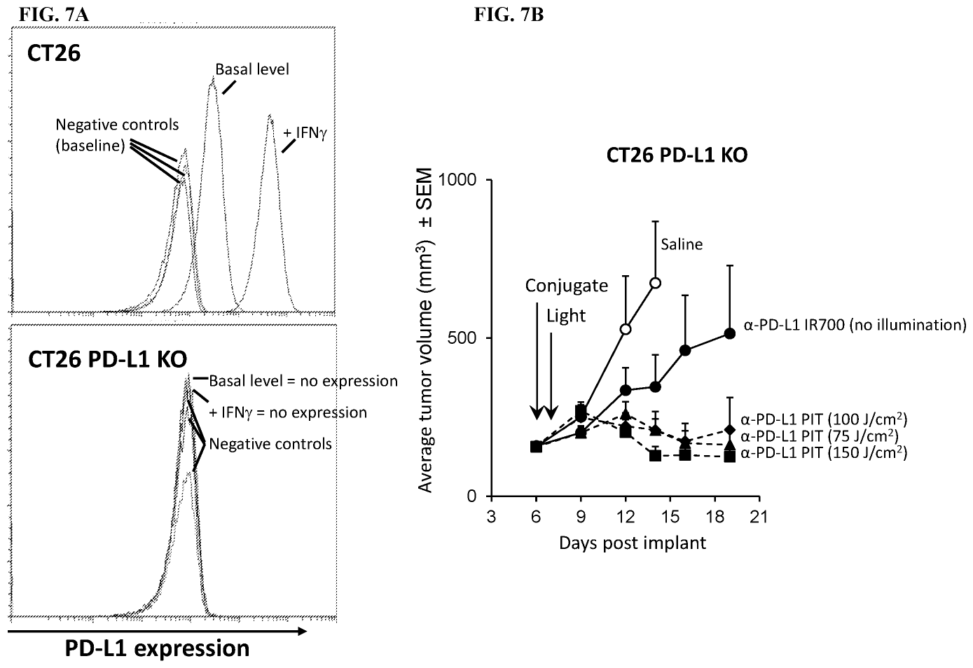

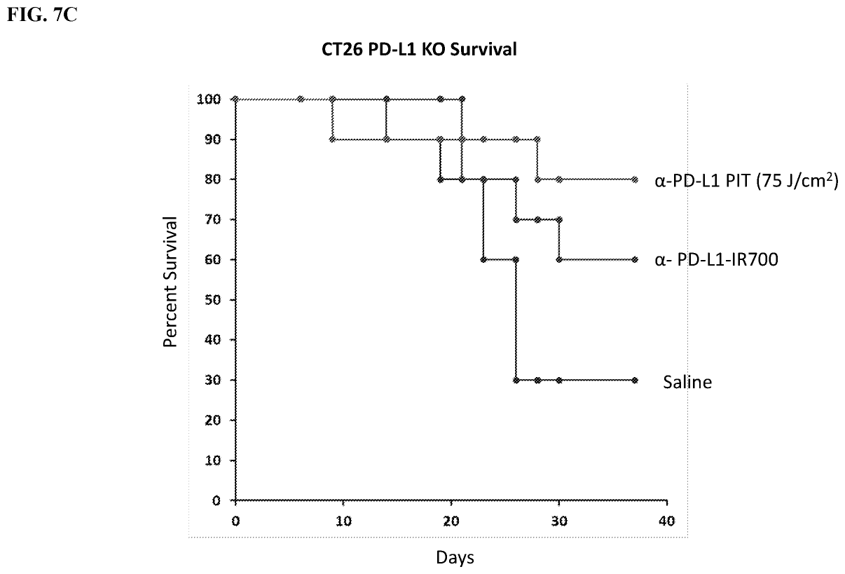

PD-L1 expression was assessed in CT26 cells with or without PD-L1 knockout (KO), in the absence (basal level) or presence of interferon gamma (IFNγ) that can induce the expression of PD-L1 in the cells. As shown in FIG. 7A, CT26 cells expressed a basal level of PD-L1, and a higher level of PD-L1 in the presence of IFNγ, as expected. In the CT26 cells that were knocked out for PD-L1 (PD-L1 KO), no PD-L1 expression was observed, either at the basal level (absence of IFNγ) or in the presence of IFNγ.

BALB/c mice at age of 6-8 weeks were inoculated with 1×10⁶ CT26 cells with PD-L1 KO subcutaneously on day 0. When the CT26 PD-L1 KO tumor grew to a size about 150 mm³ (approximately day 6 after tumor implantation) mice were administered with saline (100 μL; control) or anti-PD-L1-IR700 conjugate (100 μg) generated substantially as described in Example 1 above. Twenty-four hours after administration of the conjugate, tumors in the photoimmunotherapy (PIT) group were illuminated at 690 nm and a dosage of 75, 100 or 150 J/cm². The tumor growth was observed over approximately 21 days, and tumor volume was calculated using the formula: tumor volume =(width x width) x length/2. Survival was also monitored.

Administration of anti-PD-L1-IR700 (α-PD-L1-IR700) and illumination (PIT) at various light doses resulted in substantial tumor growth inhibition of CT26 PD-L1 KO tumors and increased survival, compared to tumor growth inhibition in control mice that received saline or anti-PD-L1-IR700 conjugate alone without PIT (FIGS. 7B and 7C). Mice receiving anti-PD-L1-IR700 conjugate alone without PIT also exhibited a reduction in tumor growth, and increased survival, compared to saline control mice (FIGS. 7B and 7C). The results showed that anti-PD-L1-IR700 PIT treatment results in substantial inhibition of growth of tumors with a PD-L1 knock-out; consistent with an observation that the anti-cancer activity of anti-PD-L1-IR700 PIT treatment is not primarily due to direct targeting and killing of cancer cells.

### Example 9: Anti-PD-L1-IR700 PIT Reduces Cell Types that Express PD-L1 In Vivo

This example describes the stimulatory effect of anti-PD-L1-IR700 PIT, on cell populations that express PD-L1 in vivo.

BALB/c mice were inoculated with CT26 tumor cells. Once tumors reached an approximate average volume of 150 mm³, mice were treated with saline, anti-PD-L1-IR700 conjugate alone or anti-PD-L1-IR700 conjugate with illumination (anti-PD-L1-IR700 PIT). Twenty-four hours after administration of the conjugate, tumors in the mice of the illuminated (PIT) group were exposed to 690 nm light at 100 J/cm². Two hours post illumination, tumors were excised from all groups and processed into single cell suspensions. Suspended cells were then stained for cell markers, including CD11b, CD11c, CD80, CD86, CD103, F4/80, Ly6C, Ly6G, and MHCII, to identify intratumoral monocytes, macrophages, neutrophils, myeloid-derived suppressor cells (MDSCs), and dendritic cells (DCs). Isotype controls were also used for staining. The stained cells were analyzed using flow cytometry.

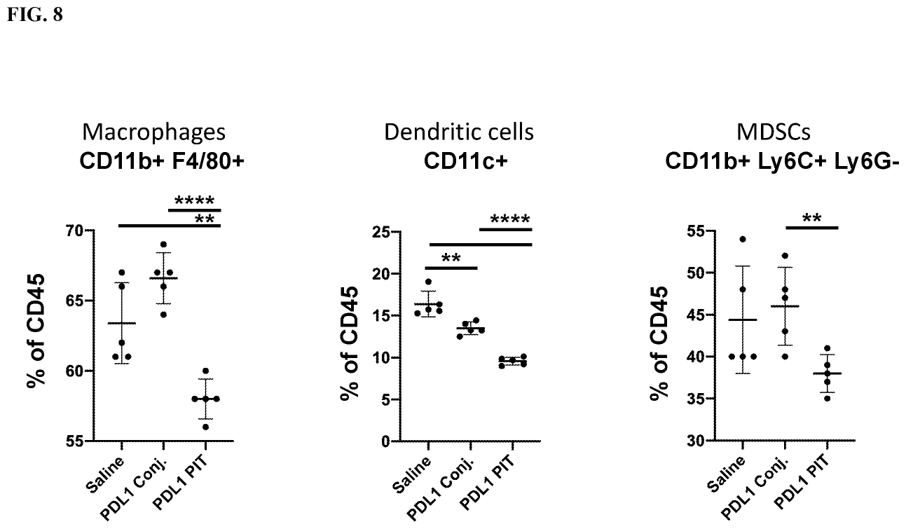

As shown in FIG. 8, in tumor-bearing mice treated with PD-L1-1R700 PIT (PDL1 PIT), the proportion of macrophages (CD11b⁺F4/80⁺ cells), dendritic cells (CD11c⁺ cells), and MDSCs (CD11b⁺ Ly6C⁺ Ly6G⁻ cells) in the tumors were significantly decreased compared to those administered only saline or anti-PD-L1-IR700 conjugate (PDL1 Conj.; without illumination), indicating anti-PD-L1-IR700 PIT immediately reduced multiple myeloid cell types post treatment. Because the myeloid cells, which are the intratumoral PD-L1-expressing immune cells, are reduced following anti-PD-L1 PIT, these data suggest that anti-PD-L1 PIT targets and kills intratumoral PD-L1 expressing immune cells.

### Example 10: Anti-PD-L1-IR700 PIT Causes Recruitment of Intratumoral Neutrophils In Vivo

The tumor cells harvested and stained in Example 9 above were also analyzed for neutrophils, identified as CD11b⁺ Cy6C^{-/low} Ly6G⁺ cells.

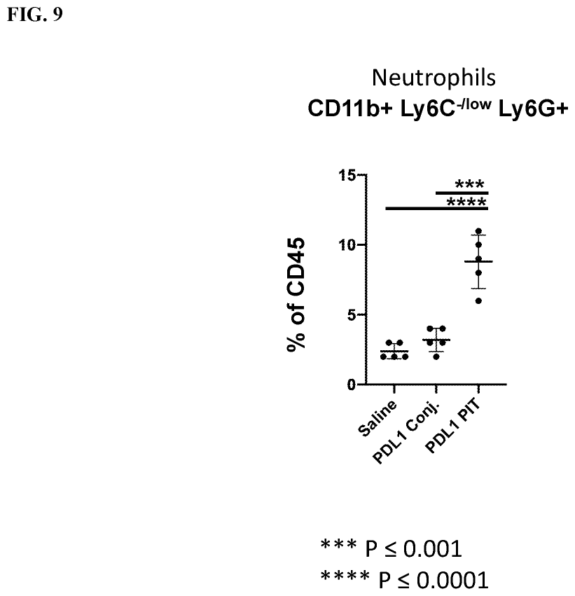

As shown in FIG. 9, in tumors of mice treated with PD-L1-1R700 PIT (PDL1 PIT), the proportion of intratumoral neutrophils (CD11b⁺ Cy6C^{-/low} Ly6G⁺ cells) was significantly increased compared to those administered only saline or anti-PD-L1-IR700 conjugate (PDL1 Conj.; without illumination). Because neutrophils are recruited to sites of inflammation, this result suggests anti-PD-L1 PIT results in rapid inflammation in the tumor.

### Example 11: Anti-PD-L1-IR700 PIT Activates the Innate Immune System In Vivo

This example describes the effect of anti-PD-L1-IR700 PIT, on dendritic cell activation in vivo.

BALB/c mice were inoculated with CT26 tumor cells. Once tumors reached an approximate average volume of 150 mm³, mice were treated with saline, anti-PD-L1-IR700 conjugate alone (PDL1 Conj.) or anti-PD-L1-IR700 conjugate with illumination (anti-PDL1 PIT). Twenty-four hours after administration of the conjugate, tumors in the mice of the illuminated (PIT) group were exposed to 690 nm light at 100 J/cm². Two days post illumination, tumors were excised and processed into single cell suspensions. Suspended cells were then stained for cell markers, including CD11b, CD11c, CD40, CD80, CD86, CD103, and MHCII, to identify intratumoral dendritic cells (DCs). Isotype controls were also used for staining. The stained cells were analyzed using flow cytometry.

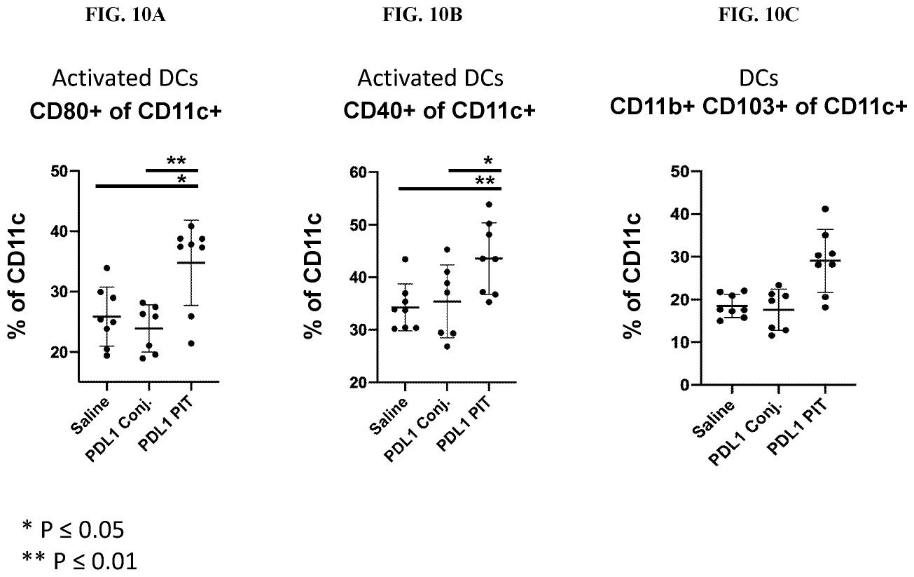

As shown in FIGS. 10A and 10B, tumors of mice treated with PD-L1-IR700 PIT (PDL1 PIT), contained significantly increased levels of activated dendritic cells, indicated by CD80⁺ (FIG. 10A) and CD40⁺ (FIG. 10B) markers, compared to tumors of mice administered only saline or anti-PD-L1-IR700 conjugate without illumination (PDL1 Conj.). CD40 and CD80 are costimulatory molecules for T cell activation, which activate naïve and memory T cells and stimulate dendritic cells by enhancing cytokine production. Tumors of mice treated with PD-L1-IR700 PIT (PDL1 PIT), also contained increased levels of antigen-presenting dendritic cells (CD103⁺ CD11c⁺) (FIG. 10C) compared to tumors of mice administered only saline or anti-PD-L1-IR700 conjugate (PDL1 Conj.; without illumination). Taken together, these data indicate anti-PD-L1-IR700 PIT activates the intratumoral innate immune response in vivo.

### Example 12: Anti-PD-L1-IR700 PIT Increases the Non-Exhausted Intratumoral Effector CD8⁺ T Lymphocytes In Vivo

This example describes the stimulatory effect of anti-PD-L1-IR700 PIT, on the expansion of effector CD8+ T lymphocytes in vivo.

BALB/c mice were inoculated with CT26 tumor cells. Once tumors reached an approximate average volume of 150 mm³, mice were treated with saline, anti-PD-L1-IR700 conjugate alone or anti-PD-L1-IR700 conjugate with illumination (anti-PD-L1-IR700 PIT). Twenty-four hours after administration of the conjugate, tumors in the mice of the illuminated (PIT) group were exposed to 690 nm light at 100 J/cm². Eight days post illumination, tumors were excised from all groups and processed into single cell suspensions. Suspended cells were then stained for cell markers including CD3, CD45, CD8a, and PD1. Isotype controls were also used for staining. The stained cells were analyzed using flow cytometry.

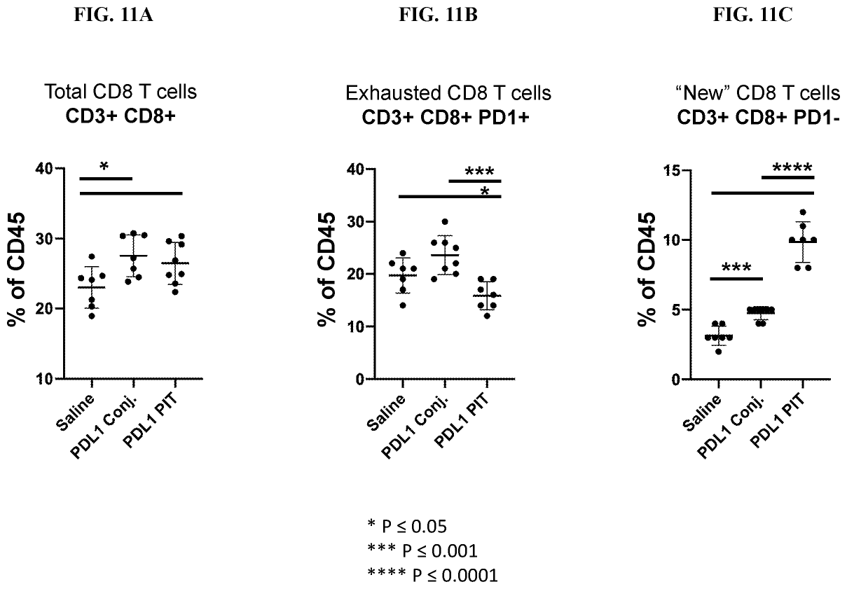

As shown in FIG. 11A, the proportion of total CD8+ T cells was increased in tumors of mice treated with anti-PD-L1 conjugate or anti-PD-L1-IR700 PIT, in comparison to tumors of mice administered only saline (P<0.05). The proportion of CD8+ T cells expressing PD1, a marker of exhaustion, was less in anti-PD-L1-IR700 PIT-treated tumors than in tumors from mice administered only saline or the anti-PD-L1 conjugate (no illumination) (FIG. 11B). In contrast, PD1−CD8+ T cells, corresponding to newly primed CD8+ T cells derived from peripheral immune activation, were increased in in anti-PD-L1-IR700 PIT-treated tumors, compared to tumors from mice administered only saline or the anti-PD-L1 conjugate (no illumination) (FIG. 11C). These data suggest that local treatment of anti-PD-L1 PIT can activate the systemic adaptive immune response.

### Example 13: Anti-PD-L1 PIT Induces Anti-Cancer Response in Distal Tumors

This example describes the inhibitory effect of anti-PD-L1 PIT on the growth of distal tumors that are not directly illuminated.

BALB/c mice were inoculated with 1×10⁶ CT26 murine colon carcinoma cells, per mouse subcutaneously on both the right and left hind flanks. When allograft tumors on both sides grew to volumes of about 150 mm³, mice were administered saline (100 μL) or anti-PD-L1-IR700 conjugate (100 μg). Twenty-four hours after administration of the conjugate, tumors in the right flank in the anti-PD-L1 PIT group were illuminated at 690 nm at a dosage of 100 J/cm², while tumors in the left flank were shielded from illumination. The growth of the non-illuminated tumor (distal tumor) was observed over 18 days, and tumor volume was calculated using the formula: tumor volume=(width×length)×height/2.

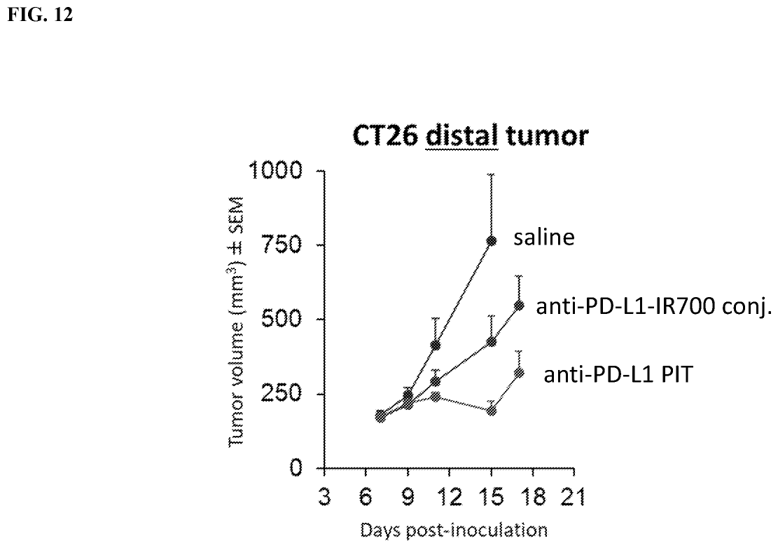

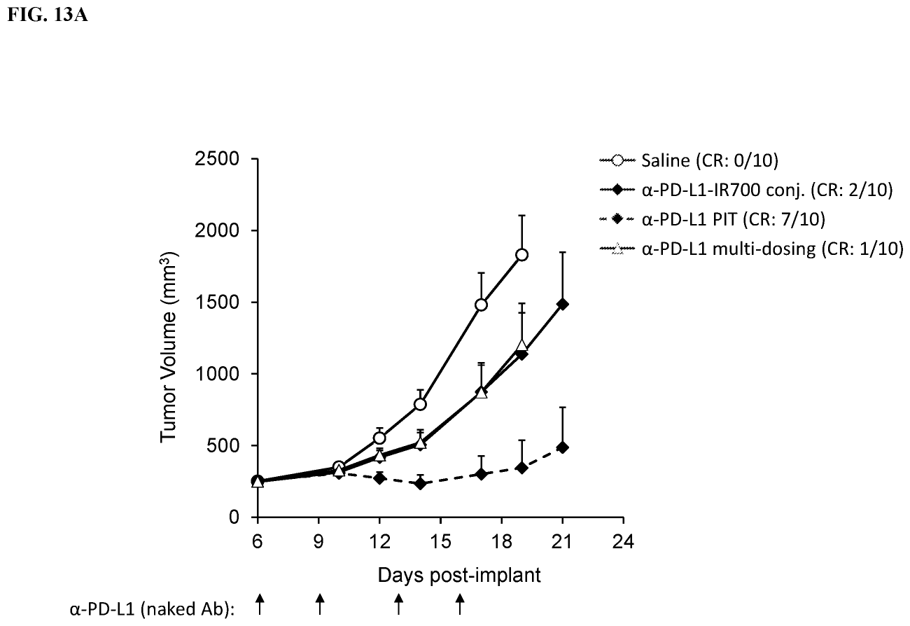

As shown in FIG. 12, non-illuminated, distal tumors in mice treated with anti-PD-L1 PIT on the contralateral side exhibited tumor growth inhibition compared to saline-treated or anti-PD-L1-IR700 conjugate-treated (no illumination) tumors. Mice administered the anti-PD-L1-IR700 conjugate alone (without illumination) also exhibited distal tumor growth compared to saline controls, but the conjugate alone was less effective in inhibiting distal tumor growth than anti-PD-L1-PIT. These data support the finding that anti-PD-L1 PIT is able to induce a systemic immune response and exhibit an abscopal effect, e.g., inhibition of distal (non-illuminated) tumor growth, in comparison to treatment with the anti-PD-L1-IR700 conjugate alone.

### Example 14: Anti-PD-L1 PIT Results in Improved Tumor Burden Reduction Compared to Multi-Dosing of Naked Anti-PD-L1 Antibody

The anti-PD-L1 antibody, Avelumab (BAVENCIO), a human anti-PD-L1 antibody that cross-reacts with mouse PD-L1, was conjugated to IR700 dye substantially as described in Example 1.

BALB/c mice (6-8 weeks of age) were inoculated with 1×10⁶ CT26 murine colon carcinoma cells subcutaneously in the right hind flank. When allograft tumors grew a size of about 250 mm³ (approximately day 6 after tumor implantation) mice were retro-orbitally administered saline (100 μL; control) or the anti-PD-L1-IR700 conjugate (anti-PD-L1-IR700; 100 μg). Twenty-four hours after administration of the conjugate, tumors in the photoimmunotherapy (PIT) group were illuminated at 690 nm at 75 J/cm². For comparison, another group of mice were administered naked (unconjugated) anti-PD-L1 antibody at 10 mg/kg twice per week for a total of 12 doses by intraperitoneal injection starting on Day 6 after implantation. The tumor growth and survival were monitored for all groups over time. Tumor volume was calculated using the formula: tumor volume =(width x width) x length/2.

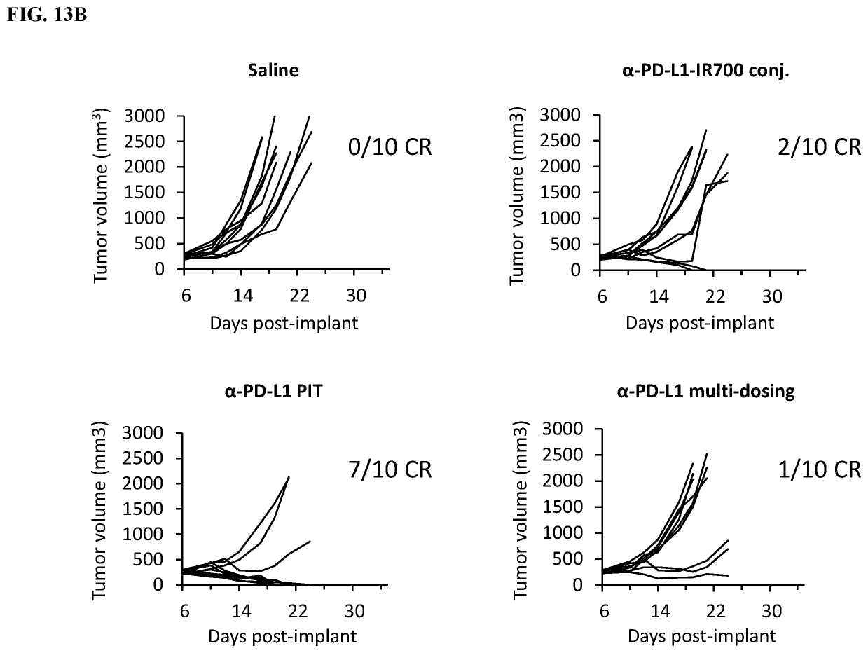

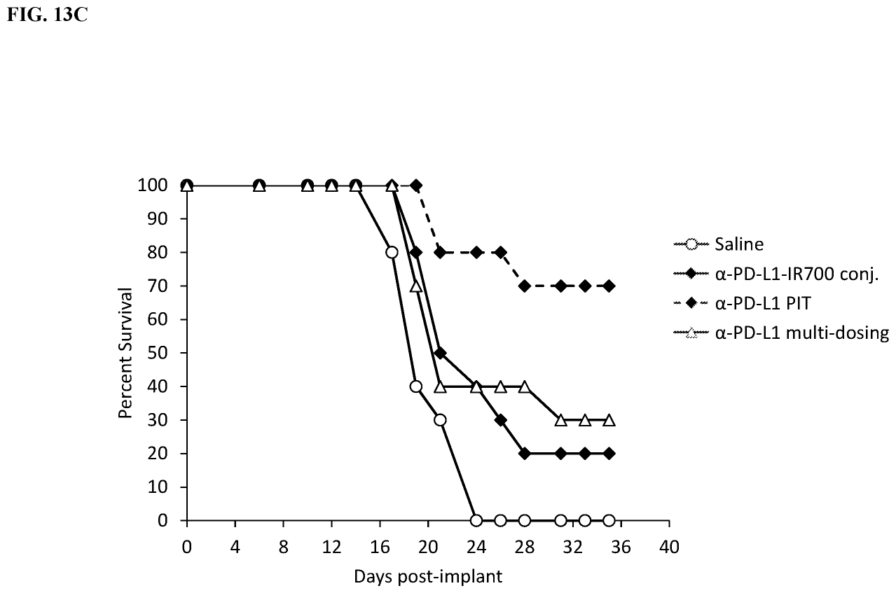

The average tumor growth for all groups of mice over time is plotted in FIG. 13A, and the tumor growth for individual mice is plotted in FIG. 13B. As shown in FIGS. 13A and 13B, in mice that received the anti-PD-L1-IR700 with illumination (α-PD-L1 PIT), the growth of tumors was substantially inhibited in comparison to tumor growth inhibition in control mice that received saline, anti-PD-L1-IR700 conjugate alone (no illumination), or multiple doses of the naked anti-PD-L1 antibody. The tumors of mice receiving multiple doses of naked anti-PD-L1 antibody exhibited a similar rate of tumor growth as tumors of mice receiving the single dose of the anti-PD-L1-IR700 conjugate, which was reduced compared to saline control mice.

In addition to examining the tumor growth, the rate of complete response (CR) was compared among the treatment groups. In this example, CR was defined as tumors with a volume of less than 100 mm³ sustained for at least 2 weeks. By the end of the study, 7 of the 10 mice treated with anti-PD-L1-IR700 PIT, 2 of the 10 mice treated with anti-PD-L1-IR700 alone (no illumination), 1 of the 10 mice treated with multiple doses of naked anti-PD-L1 antibody, and none of the 10 mice treated with saline only achieved CR (FIG. 13A).

The survival rate of tumor-bearing mice receiving anti-PD-L1-IR700 PIT achieved the highest survival of any of the treatment groups (70% survival; FIG. 13C). The survival of tumor-bearing mice receiving a single dose of anti-PD-L1 conjugate (no illumination) was similar to the survival of mice administered multiple doses of the naked anti-PD-L1 antibody (20% vs. 30%; FIG. 13C). No animals survived past 24 days in the saline group (FIG. 13C).

These results indicate that one treatment of anti-PD-L1-IR700 PIT is more efficacious in reducing tumor burden and promoting survival than multiple-cycle dosing of naked anti-PD-L1 antibody.

### Example 15: Anti-PD-L1 PIT Reduces Tumor Burden in an Immunosuppressive Murine Tumor Model

Avelumab (BAVENCIO) is an anti-PD-L1 antibody used to treat human cancers. Avelumab also cross reacts with and binds to mouse PD-L1 molecule. Anti-PD-L1-IR700 PIT (with Avelumab-IR700) was compared with anti-PD-L1-IR700 conjugate alone (no illumination) and naked anti-PD-L1 (Avelumab) for treatment of tumors resistant to anti-PD-L1 treatment.

C57B1/6 mice (6-8 weeks of age) were inoculated with 5×10⁵ LL/2 murine lung carcinoma cells subcutaneously in the right hind flank. When allograft tumors grew a size of about 150 mm³ (approximately day 8 after implantation) mice were retro-orbitally administered saline (100 μL; control) or an anti-PD-L1-IR700 conjugate (anti-PD-L1-IR700; 100 μg). Twenty-four hours after administration of the conjugate, tumors in the photoimmunotherapy (PIT) group were illuminated at 690 nm at 150 J/cm². For comparison, another group of mice were administered the naked anti-PD-L1 antibody at 10 mg/kg twice per week for a total of 6 doses by intraperitoneal injection starting on Day 8 after implantation. The tumor growth was monitored for all groups over time as described above.

In an additional experiment, mice (6-8 weeks of age), bearing LL/2 tumors, generated as described above, were administered 100 μg naked anti-PD-1 antibody, on days 6, 10, and 14, naked anti-CTLA-4 antibody, on days 6, 9, 12, and 15, or saline (100 μL; control). The tumor growth was monitored for all groups over time as previously described.

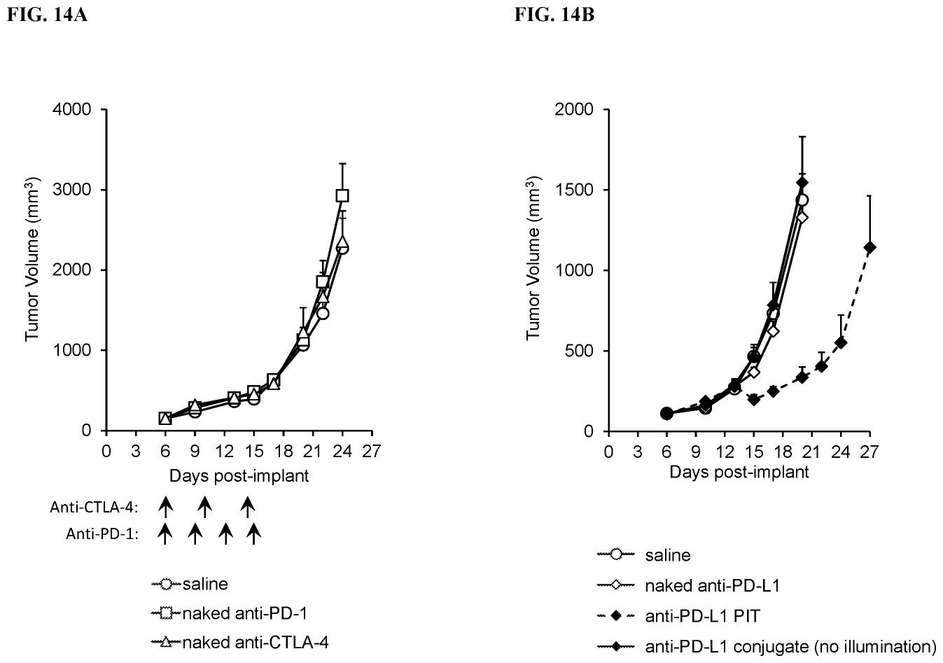

As shown in FIGS. 14A and 14B, the growth of LL/2 tumors in mice treated with naked anti-PD-1 (FIG. 14A; open squares), naked anti-CTLA-4 (FIG. 14A; open triangles), or naked anti-PD-L1 (FIG. 14B; open diamonds) was indistinguishable from tumors in mice administered saline (FIGS. 14A and 14B; open circles), indicating these tumors are resistant to these checkpoint inhibitor therapies and are immunosuppressive. In contrast, as shown in FIG. 14B, the tumors in mice that received the anti-PD-L1-IR700 with illumination (anti-PD-L1 PIT), the growth of immunosuppressive tumors was substantially inhibited in comparison to tumor growth observed in control mice that received saline, anti-PD-L1-IR700 conjugate alone (no illumination), or multiple doses of the naked anti-PD-L1 antibody (filled diamonds with dashed line vs. open circles, filled diamonds, and open diamonds with solid lines, respectively). In addition, the tumors of mice receiving multiple doses of naked anti-PD-L1 antibody or the single dose of the anti-PD-L1-IR700 conjugate (no illumination) were indistinguishable from tumors of saline-treated control mice.

Taken together, these results indicate that anti-PD-L1 PIT was effective in treating tumors that were resistant to anti-PD-L1, anti-PD-1 and anti-CTLA-4) treatments.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of treating a tumor or lesion, the method comprising:
(a) administering a conjugate comprising a phthalocyanine dye linked to a targeting molecule that binds to PD-L1 to a subject having a tumor or lesion comprising a tumor cell that is reduced in susceptibility to treatment with an immune checkpoint inhibitor; and
(b) illuminating a target area where the tumor or lesion is located in the subject, at a wavelength of at or about 600 nm to at or about 850 nm and at a dose of from at or about 25 J/cm² to at or about 400 J/cm² or from at or about 2 J/cm fiber length to at or about 500 J/cm fiber length;
wherein after the illumination, the growth, size or viability of the tumor or lesion is reduced or inhibited.

2. A method of treating a tumor or lesion, the method comprising:
(a) administering a conjugate comprising a phthalocyanine dye linked to a targeting molecule that binds to PD-L1 to a subject having a tumor or lesion that has had a low response to, was unresponsive to, was resistant to, was refractory to, had failed to respond to or has relapsed after, a prior immunotherapy; and
(b) illuminating a target area where the tumor or lesion is located, at a wavelength of at or about 600 nm to at or about 850 nm and at a dose of from at or about 25 J/cm² to at or about 400 J/cm² or from at or about 2 J/cm fiber length to at or about 500 J/cm fiber length;
wherein the method results in the killing of a PD-L1 expressing cell in the target area.

3. The method of claim 2, wherein the prior immunotherapy is a treatment with an immune checkpoint inhibitor.

4. The method of claim 2 or 3, wherein the subject has primary resistance or acquired resistance to a prior immunotherapy that comprises a PD-1/PD-L1 blockade therapy.

5. A method of treating a tumor or lesion, the method comprising:
(a) administering a conjugate comprising a phthalocyanine dye linked to a targeting molecule that binds to PD-L1 to a subject that is treatment-naïve for an immune checkpoint inhibitor or that has not previously received a treatment with an immune checkpoint inhibitor; and
(b) illuminating a target area where a tumor or lesion is located in the subject at a wavelength of at or about 600 nm to at or about 850 nm and at a dose of from at or about 25 J/cm² to at or about 400 J/cm² or from at or about 2 J/cm fiber length to at or about 500 J/cm fiber length; wherein after the illumination, the growth, size or viability of the tumor or lesion is reduced or inhibited.

6. The method of any of claims 1-5, wherein the subject is administered the conjugate to treat, inhibit the growth of and/or reduce the size of a first tumor or lesion; and the method inhibits, delays or prevents the appearance, growth or establishment of one or more second tumors or lesions, located distally to the first tumor or lesion.

7. A method of immunizing a subject having a first tumor or lesion, the method comprising:
(a) administering a conjugate comprising a phthalocyanine dye linked to a targeting molecule that binds to PD-L1 to a subject having a tumor or lesion; and
(b) illuminating a target area within the first tumor or lesion at a wavelength of at or about 600 nm to at or about 850 nm and at a dose of from at or about 25 J/cm² to at or about 400 J/cm² or from at or about 2 J/cm fiber length to at or about 500 J/cm fiber length;
wherein the first tumor or lesion is inhibited in growth and/or reduced in size; and the appearance, growth or establishment of one or more second tumors or lesions, located distally to the treated first tumor or lesion, is inhibited, delayed or prevented.

8. The method of claim 6 or 7, wherein the second tumor or lesion is a metastasis of the first tumor or lesion.

9. The method of any of claims 6-8, wherein the method results in killing of a PD-L1-expressing cell in the vicinity of the first tumor or lesion and/or activates an immune cell response, thereby inhibiting, delaying or preventing the appearance, growth or establishment of the second tumor or lesion.

10. The method of any of claims 6-9, wherein the second tumor or lesion is phenotypically and/or genotypically the same as the first tumor or lesion.

11. The method of any of claims 6-9, wherein the second tumor or lesion is phenotypically and/or genotypically different from the first tumor or lesion.

12. The method of claim 6 or 7, wherein the second tumor or lesion is not derived from a metastasis of the first tumor or lesion.

13. The method of any of claims 1-12, wherein the method results in the killing of the PD-L1-expressing cell or the PD-L1-expressing immune cell.

14. The method of any of claims 1-13, wherein the tumor or lesion comprises a tumor cell, and the tumor cell does not express or has a reduced expression of an immune checkpoint protein.

15. The method of claim 14, wherein the immune checkpoint protein is selected from among PD-L1, PD-1, and CTLA-4.

16. The method of claim 14 or 15, wherein the tumor cell does not express PD-L1 in response to an inflammatory stimulus.

17. The method of claim 16, wherein the inflammatory stimulus is interferon.

18. The method of any of claims 14-17, wherein the tumor cell is not specifically recognized by an anti-PD-L1 antibody.

19. The method of any of claims 1-18, wherein the tumor or lesion comprises PD-L1 negative tumor cells.

20. The method of claim 19, wherein at least or at least about 40%, 50%, 60%, 70%, 80%, 90% or 95% of the tumor cells in the tumor or lesion are PD-L1 negative tumor cells.

21. The method of any of claims 1-20, wherein the treatment delays regrowth of the tumor or lesion, prevents a relapse of a cancer associated with the tumor or lesion or prolongs the duration of remission of a cancer associated with the tumor or lesion.

22. The method of any of claims 1-21, wherein the inhibition of the growth of the tumor or lesion and/or killing of the PD-L1-expressing cell is dependent on the presence of CD8+ T cells.

23. The method of any of claims 1 and 6-22, wherein the subject is naïve to treatment with an immune checkpoint inhibitor or has not previously received treatment with an immune checkpoint inhibitor.

24. The method of any of claims 1, 2 and 6-22, wherein the subject has been previously treated with an immune checkpoint inhibitor.

25. The method of claim 24, wherein the subject has had a low response to, was unresponsive to, was resistant to, was refractory to, had failed to respond to or has relapsed after the previous treatment with the immune checkpoint inhibitor.

26. The method of claim 24 or 25, wherein the inhibition of the growth, size or viability of the tumor or lesion resulting from carrying out the method is greater compared to the inhibition resulting from the previous treatment with the immune checkpoint inhibitor.

27. The method of any of claims 24-26, wherein the immune checkpoint inhibitor is an inhibitor of PD-L1, PD-1 or CTLA-4.

28. The method of any of claims 24-27, wherein immune checkpoint inhibitor is a PD-1 inhibitor.

29. The method of claim 28, wherein the PD-1 inhibitor is an anti-PD-1 antibody.

30. The method of any of claims 24-27, wherein the immune checkpoint inhibitor is PD-L1 inhibitor.

31. The method of claim 30, wherein the PD-L1 inhibitor is an anti-PD-L1 antibody.

32. The method of any of claims 1-31, wherein the method increases the number or activity of immune cells in the tumor or lesion and/or in the microenvironment of the tumor or lesion.

33. The method of any of claims 1-32, wherein the target area comprises immune cells expressing PD-L1.

34. The method of any of claims 2-32, wherein the PD-L1 expressing cell is an immune cell.

35. The method of claim 33 or 34, wherein the immune cell is selected from the group consisting of monocytes, macrophages, dendritic cells (DC), M2 tumor associated macrophages (M2 TAM), tolerogenic dendritic cells (tDC) and myeloid derived suppressor cells (MDSC).

36. The method of any of claims 33-35, wherein the immune cell is located in the tumor, the tumor microenvironment or a lymph node.

37. The method of any of claims 1-36, wherein prior to administering the conjugate, the subject has a tumor or lesion having a low number or level of CD8+ T cell infiltration.

38. The method of any of claims 1-37, wherein the number, level or activity of immune cells is increased in the tumor or lesion or in the microenvironment of the tumor or lesion after the administering and the illuminating.

39. The method of claim 37 or 38, wherein the number or level of CD8+ T cell infiltration in the tumor or lesion is increased after the administering and the illuminating.

40. The method of any of claims 37-39, wherein the number or level of memory T cells in the vicinity of the tumor or lesion is increased after the administering and the illuminating.

41. The method of any of claims 1-40, wherein the targeting molecule is or comprises an antibody, an antigen-binding antibody fragment or antibody-like molecule that binds PD-L1.

42. The method of claim 41, wherein the targeting molecule is or comprises an anti-PD-L1 antibody or antigen-binding fragment thereof.

43. The method of claim 42, wherein the antibody or antigen-binding fragment comprises complementary determining regions (CDRs) from an antibody selected from the group consisting of atezolizumab (MPDL3280A, Tecentriq, RG7446), avelumab (Bavencio), BCD-135, BGB-A333, BMS-936559 (MDX-1105), CBT-502 (TQB-2450), cosibelimab (CK-301), CS1001 (WPB3155), durvalumab (MEDI4736, Imfinzi), FAZ053, HLX20, INBRX-105, KN035, KN046, LDP, LY3300054, LY3415244, M7824 (MSB0011359C), MCLA-145, MSB2311, NM-01, REGN3504, SHR-1316 (HTI-1088), STI-3031 (IMC-001, STI-A1015), TG-1501, and ZKABOO1 (STI-A1014).

44. The method of claim 42 or 43, wherein the antibody or antigen-binding fragment comprises complementary determining regions (CDRs) from atezolizumab, avelumab, durvalumab, KN035 or CK-301.

45. The method of any of claims 42-44, wherein the antibody or antigen-binding fragment is selected from the group consisting of atezolizumab, avelumab, durvalumab, KN035, and CK-301, or a biosimilar, interchangeable, biobetter, copy biological or biogeneric thereof, or an antigen-binding fragment thereof.

46. The method of any of claims 42-45, wherein the antibody or antigen-binding fragment is selected from the group consisting of atezolizumab, avelumab, durvalumab, KN035, and CK-301.

47. The method of any of claims 1-46, wherein the target area is a lymph node or in the vicinity of a lymph node.

48. The method of any of claims 1-47, wherein the subject exhibits a durable response, prolonged progression-free survival, a reduced chance of relapse, and/or a reduced chance of metastasis, after the administering and the illuminating.

49. The method of any of claims 1-48, wherein the phthalocyanine dye is a Si-phthalocyanine dye.

50. The method of claim 49, wherein the Si-phthalocyanine dye is IR700.

51. The method of any of claims 1-50, wherein the illuminating is carried out between 30 minutes and 96 hours after administering the conjugate.

52. The method of any of claims 1-51, wherein the illuminating is carried out 24 hours ±4 hours after administering the conjugate.

53. The method of any of claims 1-52, wherein the target area is illuminated at a wavelength of 690±40 nm.

54. The method of any of claims 1-53, wherein the target area is illuminated at a dose of at or about of 50 J/cm² or at or about 100 J/cm of fiber length.

55. The method of any of claims 1-54, wherein the tumor or lesion is associated with a cancer selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, gastrointestinal cancer, stomach cancer, cancer of the small intestine, spindle cell neoplasm, hepatic carcinoma, liver cancer, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

56. The method of any of claims 1-55, wherein one or more of steps of the method are repeated.

57. The method of claim 56, wherein the administration of the conjugate is repeated one or more times, optionally wherein after each repeated administration of the conjugate, the illuminating step is repeated.

58. The method of any of claims 1-57, further comprising administering an additional therapeutic agent or anti-cancer treatment.
